(19) United States
(12) Patent Application Publication
(10) Pub. No.: US 2025/0017866 A1
(43) Pub. Date: Jan. 16, 2025

(54) POLYMERS AND NANOPARTICLES FOR INTRAMUSCULAR NUCLEIC ACID DELIVERY

(71) Applicant: The Johns Hopkins University, Baltimore, MD (US)

(72) Inventors: Jordan J. GREEN, Woodstock, MD (US); David WILSON, Smithsburg, MD (US); Stephany Yi TZENG, Somerville, MA (US); Yuan RUI, Baltimore, MD (US); Sarah Y. NESHAT, Baltimore, MD (US); Kathryn M. LULY, Baltimore, MD (US)

(21) Appl. No.: 18/684,083

(22) PCT Filed: Nov. 1, 2022
(86) PCT No.: PCT/US2022/079039

§ 371 Date: Feb. 15, 2024

Related U.S. Application Data
(60) Provisional application No. 63/274,361, filed on Nov. 1, 2021.

(51) Int. Cl. A61K 9/51; A61K 9/00; A61K 47/34

(52) U.S. Cl. CPC A61K 9/51; A61K 9/0019; A61K 47/34

(57) ABSTRACT

Biodegradable cationic polyesters for intramuscular delivery of nucleic acids, including self-amplifying mRNA, and methods of their use for treating conditions or diseases are disclosed.

### STATEMENT OF GOVERNMENTAL INTEREST

This invention was made with government support under grants CA246699, CA228133, and EB028239 awarded by the National Institutes of Health. The government has certain rights in the invention.

### BACKGROUND

Vaccines are one of the few strategic tools available for wide-spread utilization in combatting infectious disease epidemics and global pandemics. Yet, traditional vaccine development of live attenuated, inactivated or subunit vaccines typically requires years of development and production time for wide-spread distribution. Parums, 2021. Genetic vaccines using mRNA to encode pathogenic antigens are one of the most promising advancements in vaccine development strategies, as they allow for rapid development of functional vaccine candidates as soon as the sequence of the desired protein target is known. Blakney et al., 2021. This approach has been adopted with great efficiency by Modema and BioNTech amongst others in response to the COVID-19 pandemic with great efficacy. Polack et al., 2020; Baden et al., 2021. Self-amplifying mRNA (SAM) technology is an innovative vaccine platform for high expression of a target antigen. Brito et al., 2015. In addition to the antigen gene of interest, SAM also encodes four alphavirus derived non-structural proteins, which constitute the replication complex responsible for self-amplification of the original mRNA in the cytosol. Brito et al., 2015; Geall et al., 2012. During the self-amplification process double-stranded RNA replication intermediates are formed and contribute to the induction of a robust Type I interferon response, that can be highly beneficial for eliciting a strong B cell response. Brito et al., 2015. Due to its self-amplifying nature, SAM vaccines have the potential for high potency and dose-sparing, which could result in the production of a higher number of vaccine doses at an equivalent amount of mRNA when compared to conventional mRNA vaccines. Vogel et al., 2018; Pardi et al., 2020. These features make SAM a platform optimal for vaccination and its utilization has been previously demonstrated to elicit protective immunity in different preclinical models, including mice and non-human primates against viral pathogens, including rabies, Blakney et al., 2019; Lou et al., 2020, influenza, Chahal et al., 2016, Ebola, Chahal et al., 2016, and HIV. Bogers et al., 2015. The biggest challenge associated with SAM is to achieve effective cytosolic delivery, as mRNA is highly susceptible to nuclease damage, which would eliminate its ability to self-amplify. Lou et al., 2020; Anderluzzi et al., 2020.

Delivery of SAM with gene delivery platforms including lipid nanoparticles, Lou et al., 2020, Geall et al., 2012, polymeric dendrimers, Chahal et al., 2016, and cationic nanoemulsions Archer et al., 2015, demonstrated varying degrees of success, but each strategy used to-date has drawbacks. Importantly, requirements for cold storage, and in some cases, the non-degradability of a mixture of synthetic components pose a challenge. Slow degradability, such as with polymeric dendrimers based on polyamidoamine modified with alkyl-epoxides, may also create issues such as inducing inflammation in vivo. Chahal et al., 2016.

### SUMMARY

In some aspects, the presently disclosed subject matter provides a nanoparticle comprising a compound of formula (I) and one or more nucleic acids:

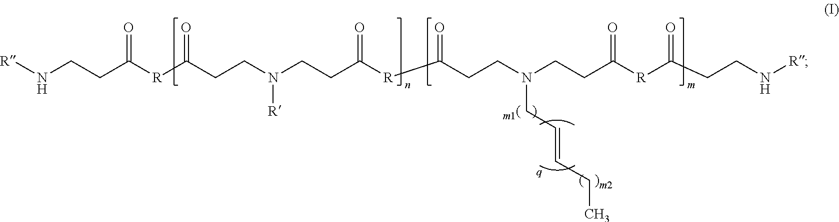

wherein: m and n are each independently an integer from 1 to 10,000; m1 is an integer selected from 0, 1, 2, 3, 4, 5, 6, 7, 8, 9, and 10; m2 is an integer selected from the group consisting of 1, 2, 3, 4, 5, 6, 7, 8, 9, 10, 11, 12, 13, 14, 15, 16, 17, 18, 19, and 20; q is an integer selected from 0 or 1; wherein —(CH₂)ₘ₁—(C═C)_{q}—(CH₂)ₙ₂—CH₃ comprises a hydrophobic sidechain; R comprises a divalent radical comprising a biodegradable ester linkage and/or a bioreducible disulfide linkage; R′ is hydrophilic sidechain comprising a monovalent radical derived from a hydrophilic amine monomer; R″ is monovalent radical derived from an amine-containing end capping group; and pharmaceutically acceptable salts thereof.

In certain aspects, R is selected from the group consisting of:

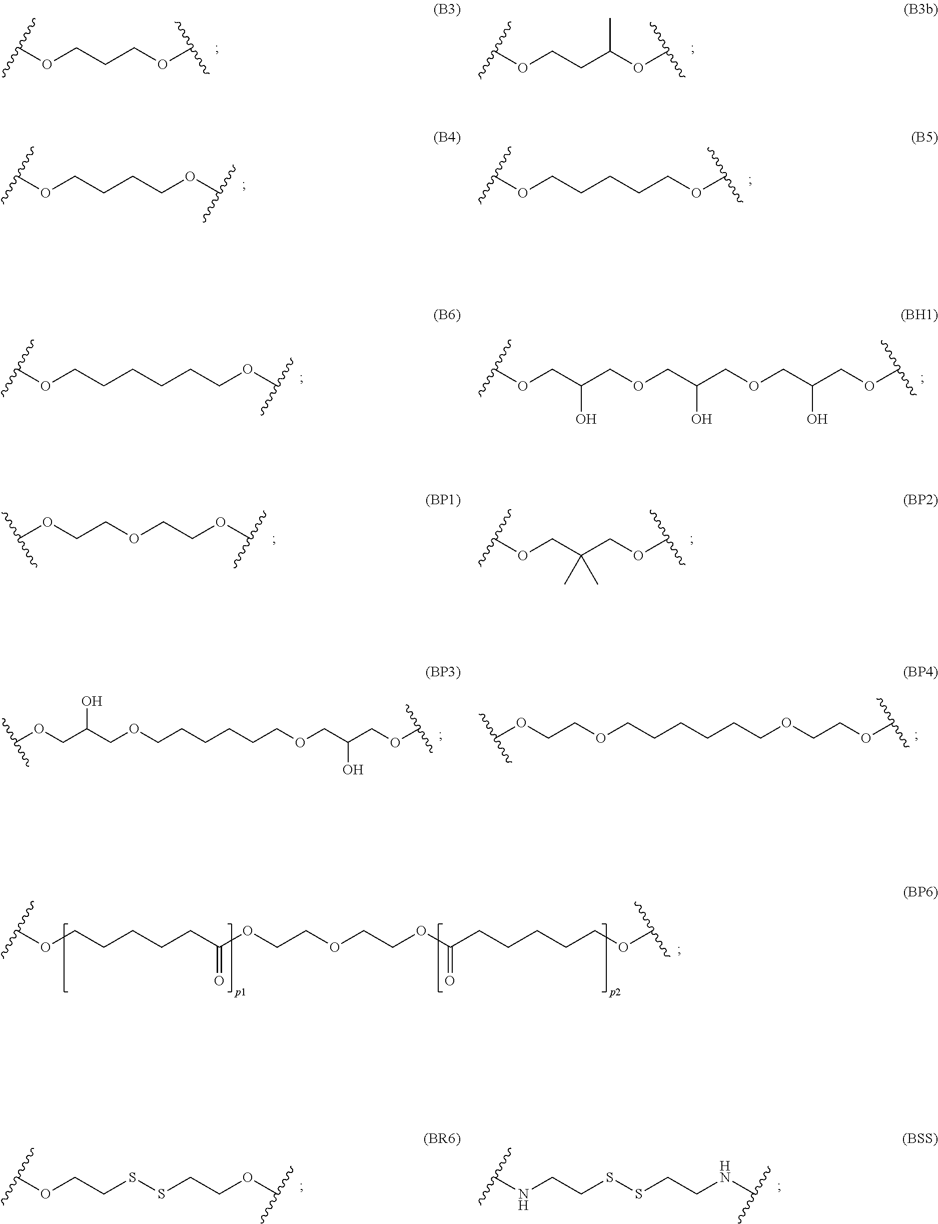

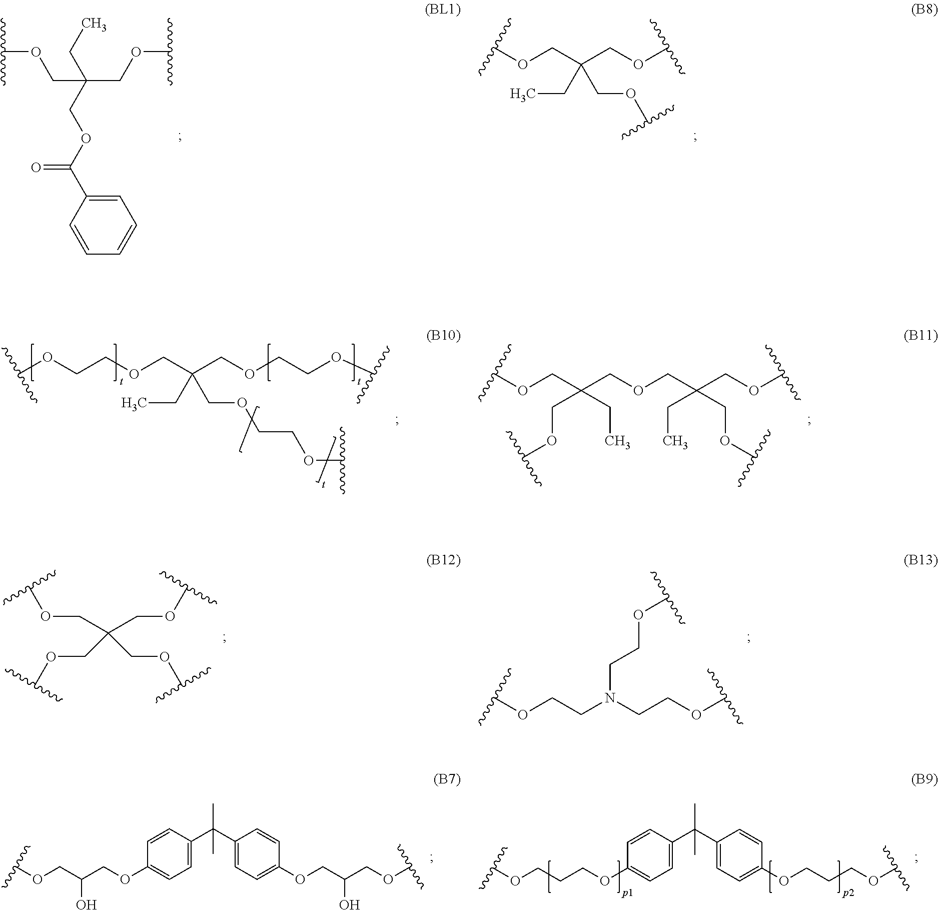

wherein each p1, p2, and t is independently an integer selected from the group consisting of 1, 2, 3, 4, 5, 6, 7, 8, 9, and 10.

In certain aspects, R′ is selected from the group consisting of:

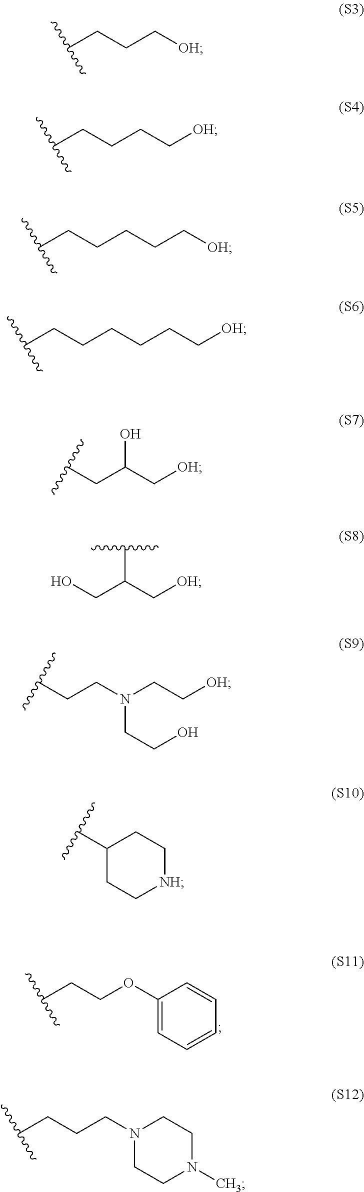

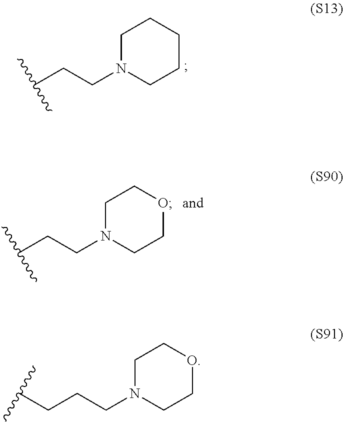 [SMILES: *CCN1CCCCC1 ; *CCN1CCOCC1 ; "and" ; *CCCN1CCOCC1]

In certain aspects, R″ is selected from the group consisting of:

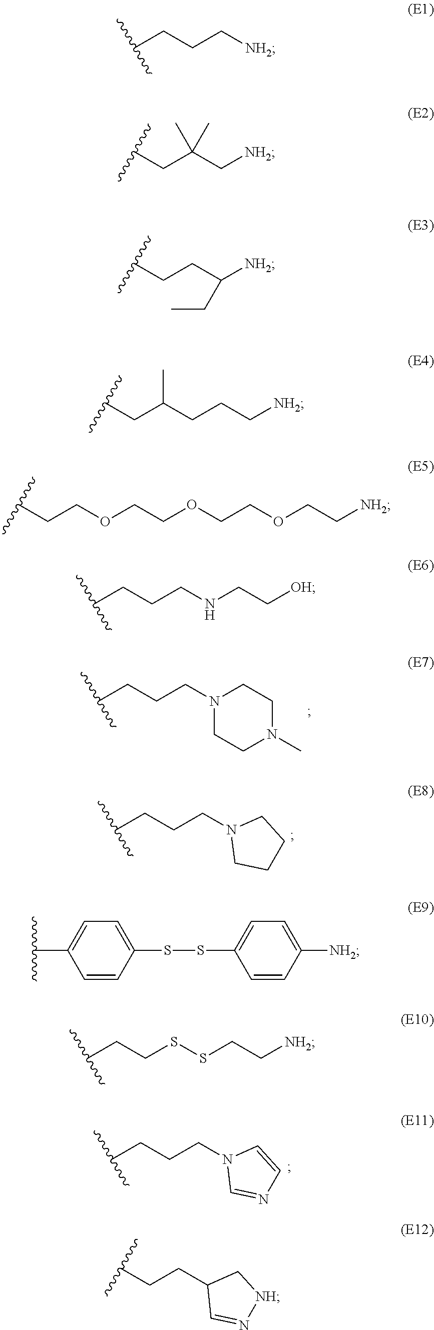

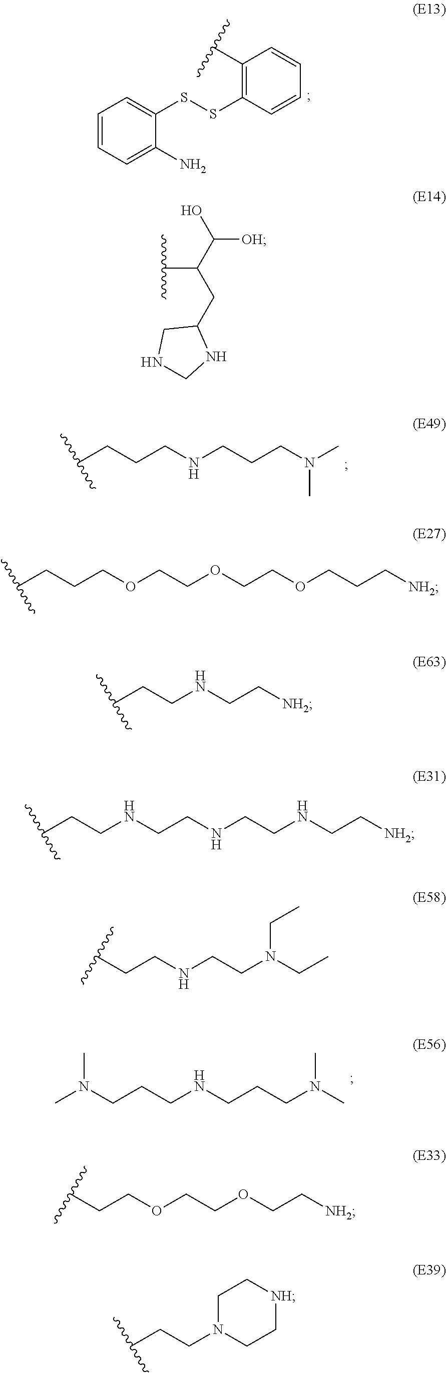

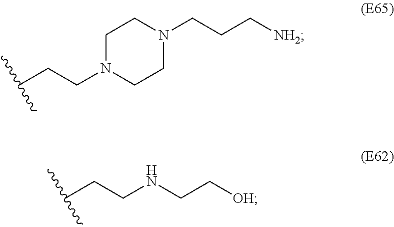 [SMILES: *CCN1CCN(CCCN)CC1 ; *CCNCCO]

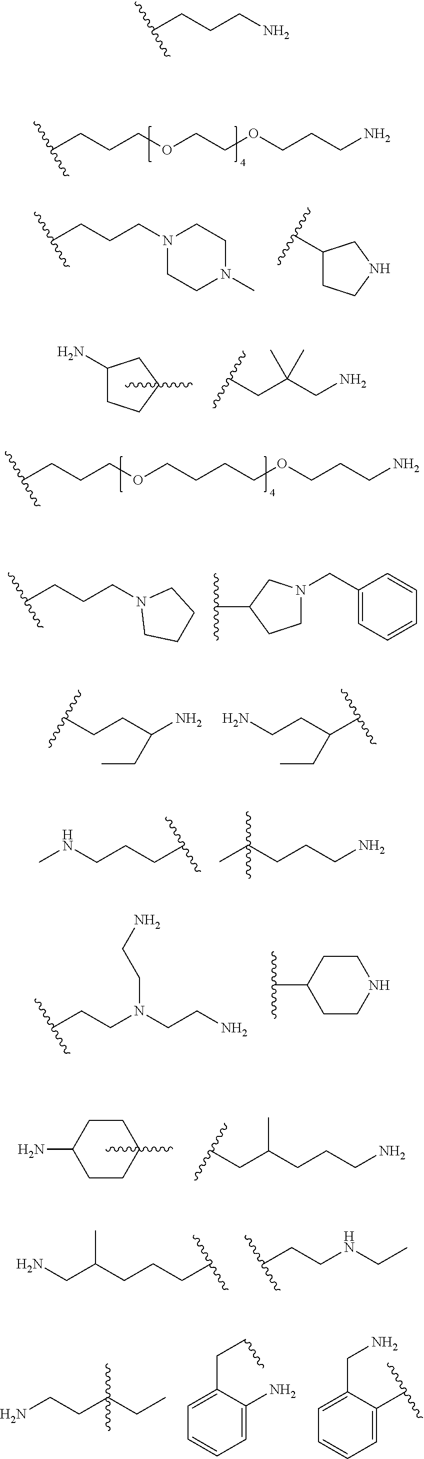

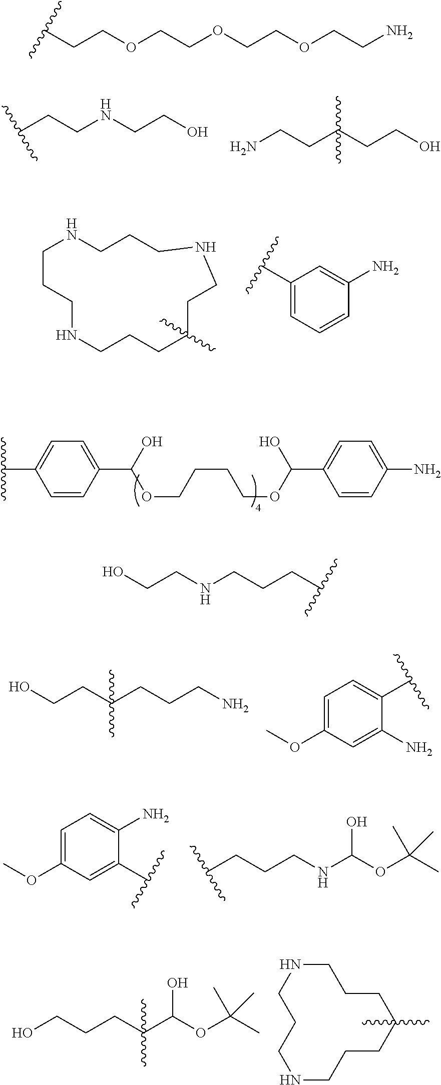

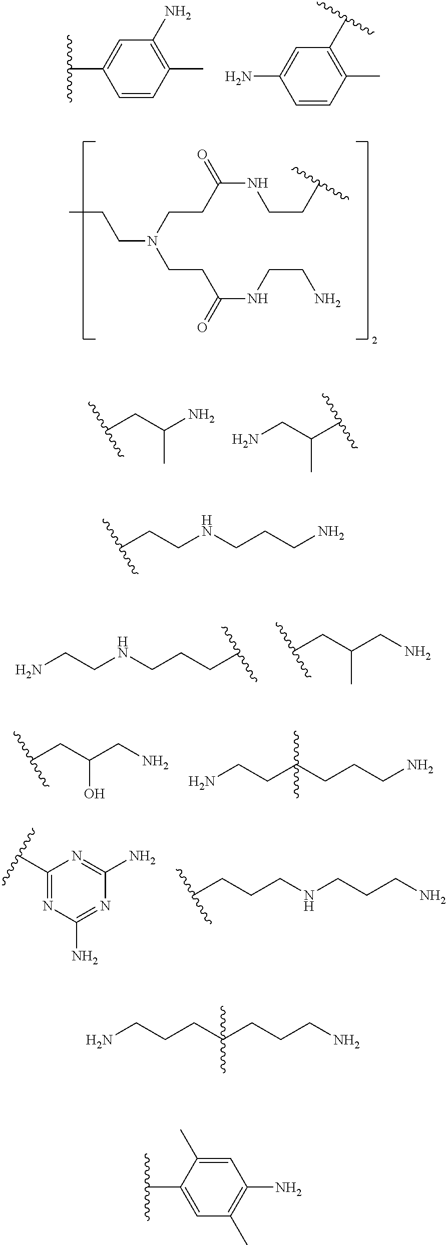

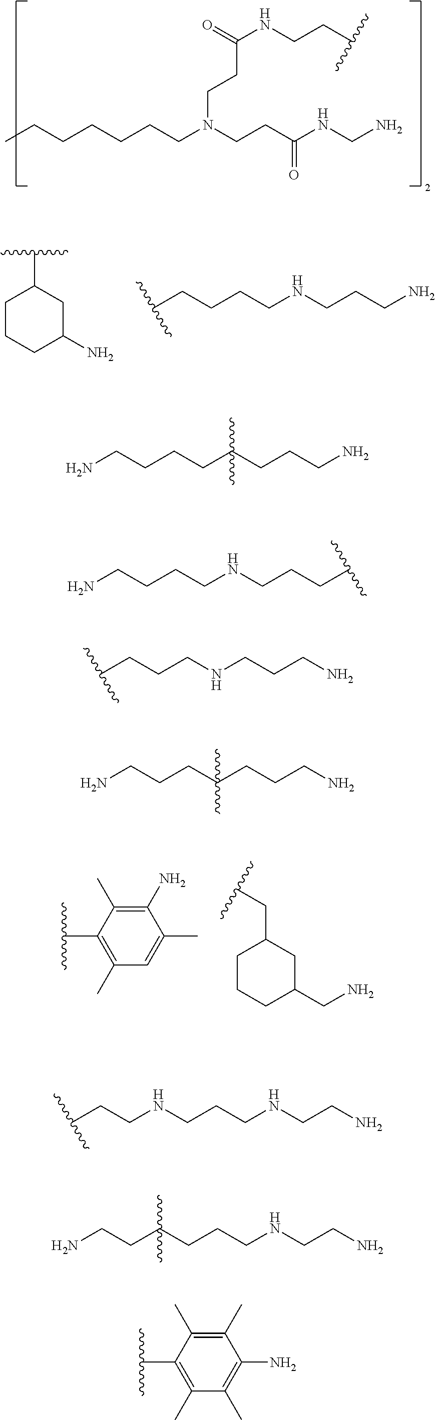

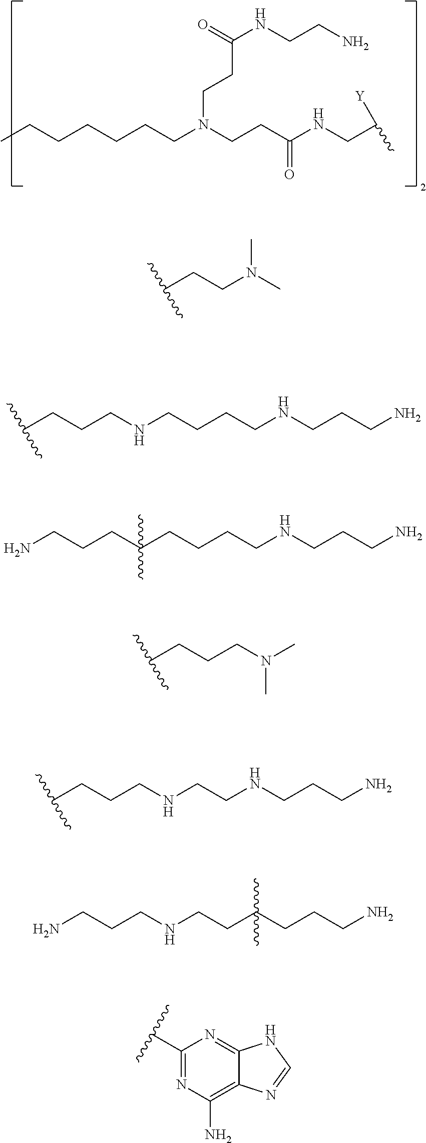

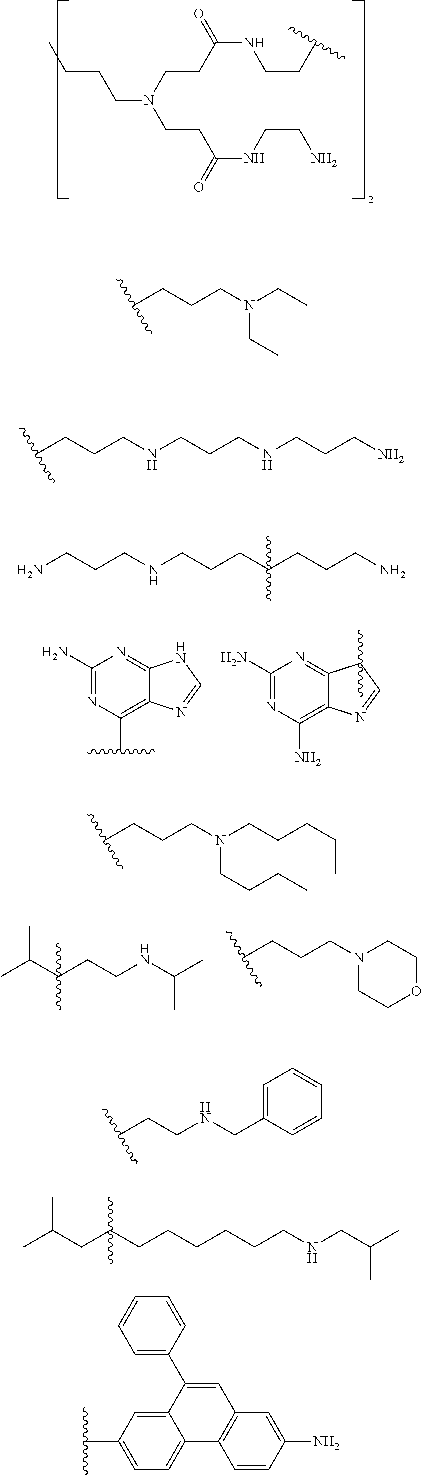

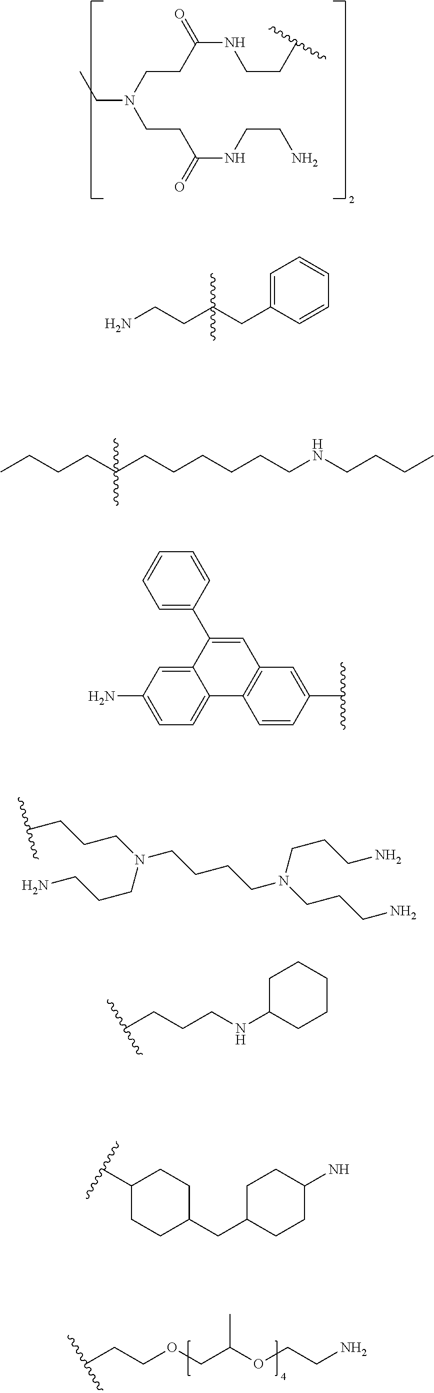

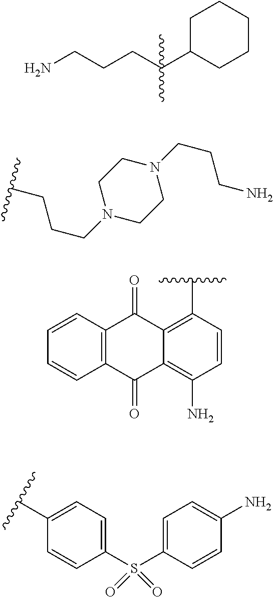

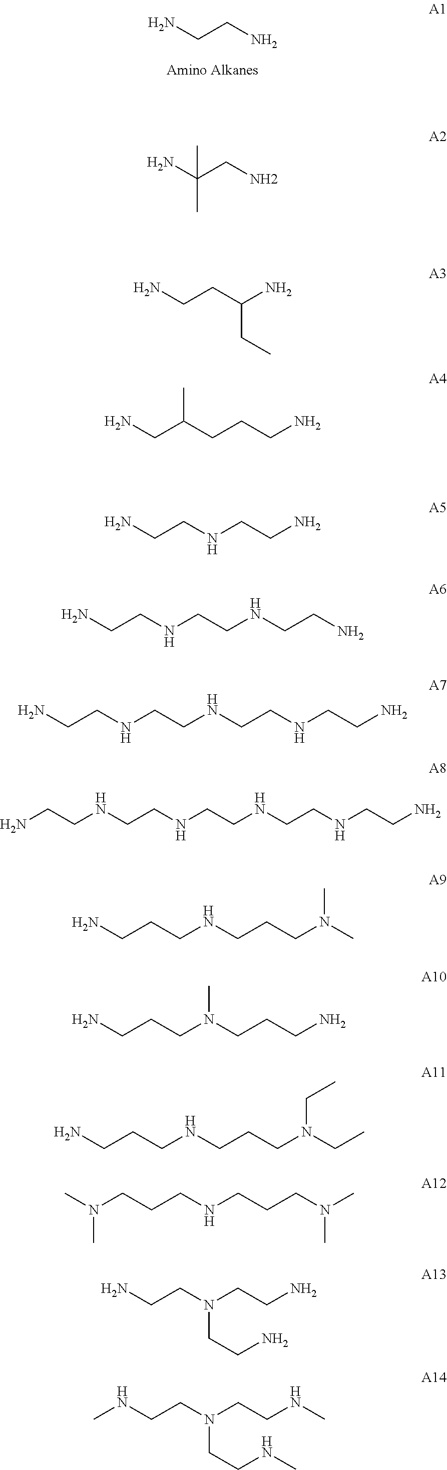

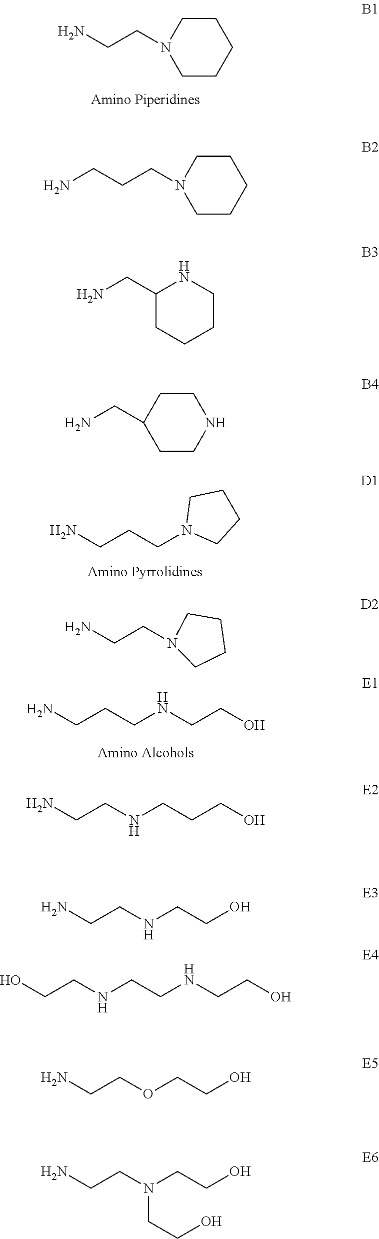

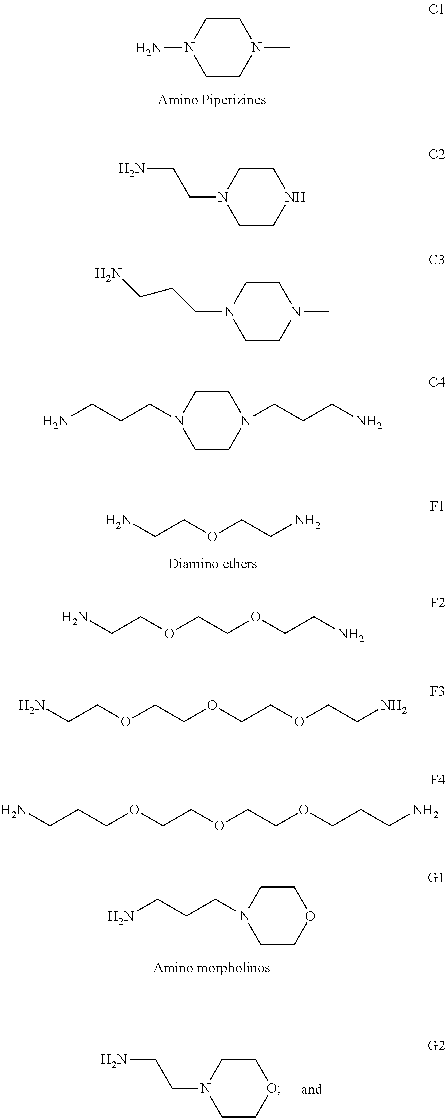

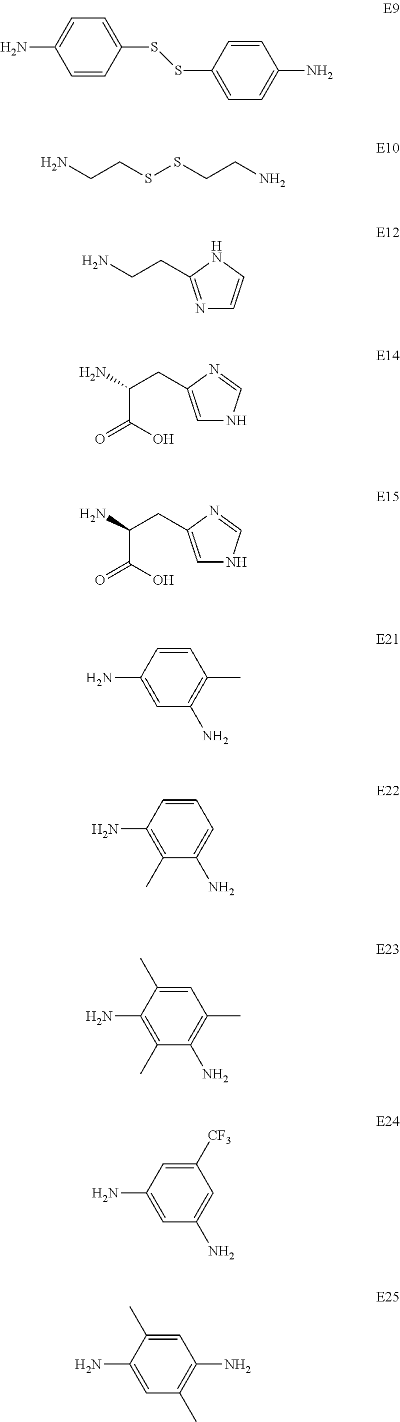

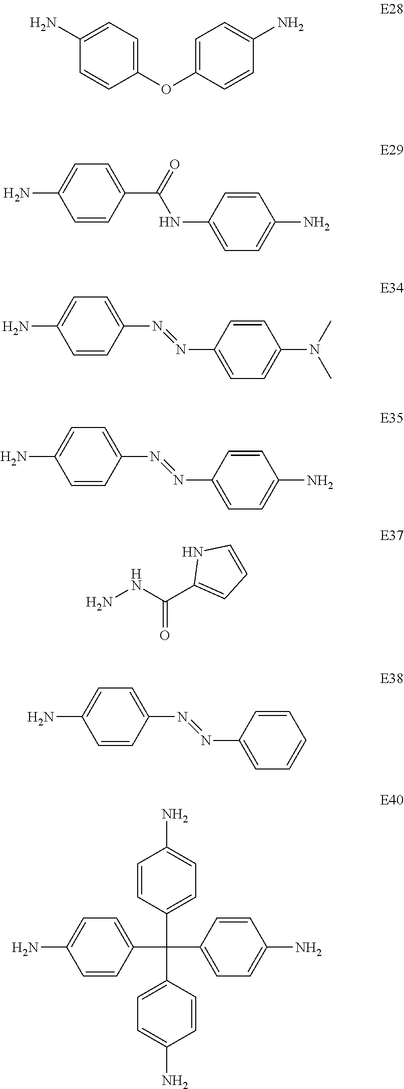

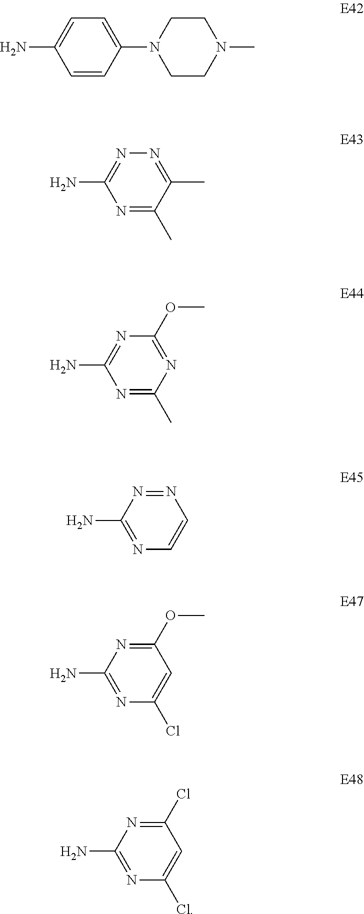

In certain aspects, —(CH₂)ₘ₁—(C═C)_{q}—(CH₂)ₘ₂—CH₃ is selected from the group consisting of

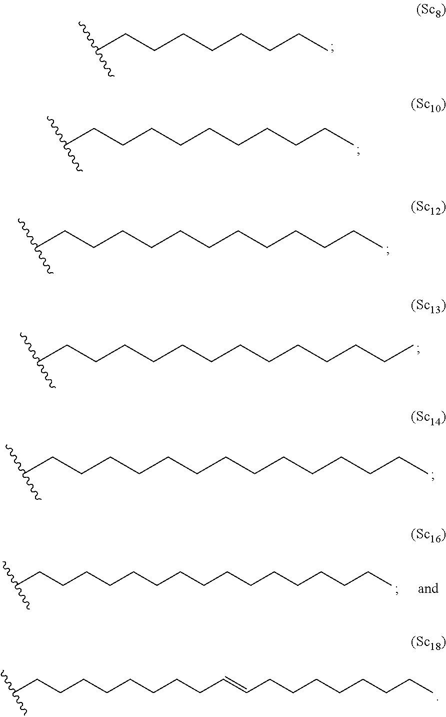

In particular aspects R is selected from the group consisting of:

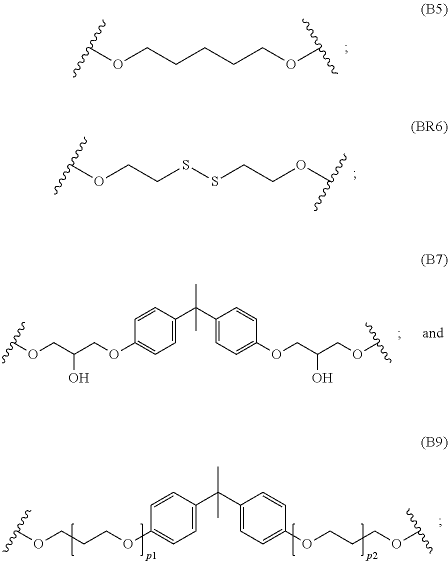

wherein each p1, p2, and t is independently an integer selected from the group consisting of 1, 2, 3, 4, 5, 6, 7, 8, 9, and 10.

In particular aspects, R′ is selected from the group consisting of:

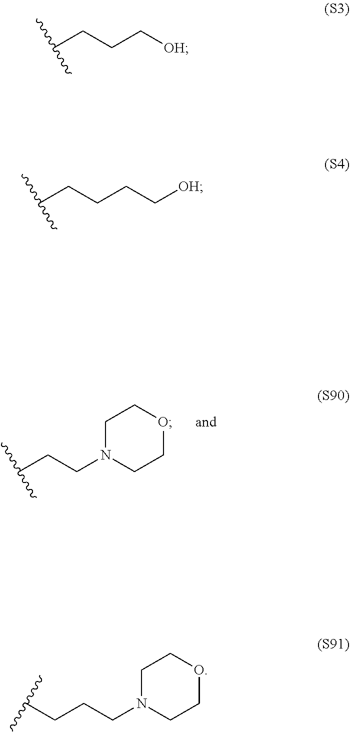 [SMILES: *CCCO ; *CCCCO ; *CCN1CCOCC1 ; "and" ; *CCCN1CCOCC1]

In particular aspects, R″ is selected from the group consisting of:

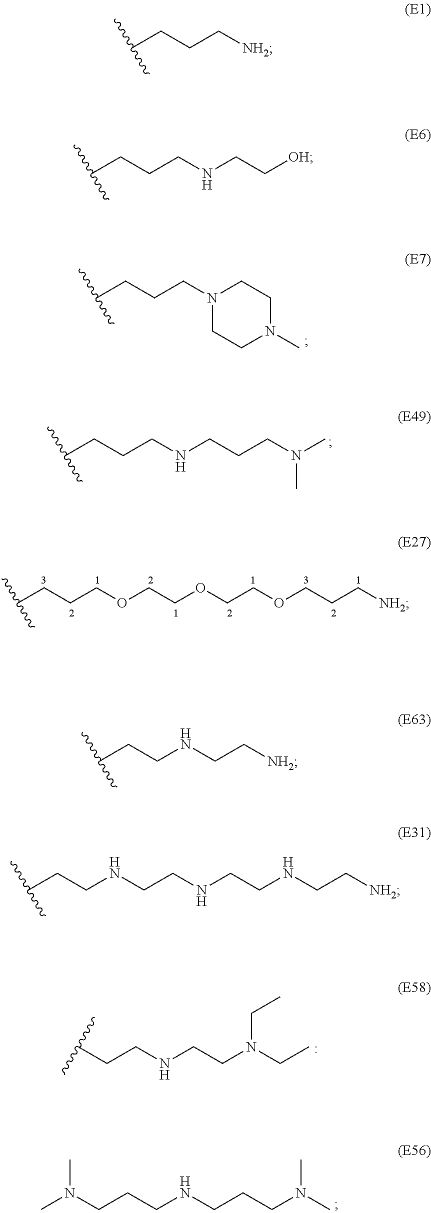

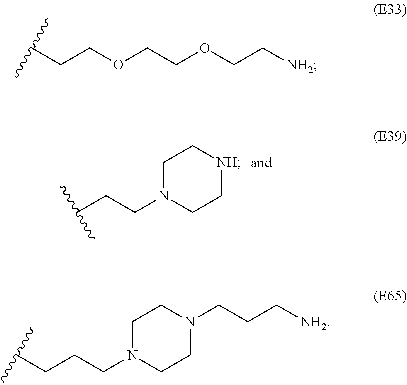 [SMILES: *CCOCCOCCN ; *CCN1CCNCC1 ; "and" ; *CCCN1CCN(CCCN)CC1]

In particular aspects, —(CH₂)ₘ₁—(C═C)_{q}—(CH₂)ₘ₂—CH₃ is selected from the group consisting of:

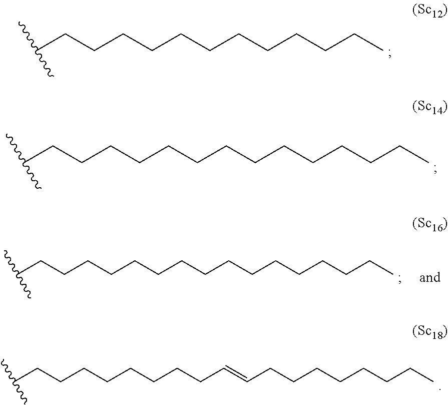 [SMILES: *CCCCCCCCCCCC ; *CCCCCCCCCCCCCC ; *CCCCCCCCCCCCCCCC ; *CCCCCCCC/C=C/CCCCCCCC]

In more particular aspects, the compound of formula (I) comprises a combination of: (a) an R group selected from the group consisting of B5, B7, B9, and BR6; (b) an R′ group selected from the group consisting of S3, S4, S90, and S91; (c) an R″ group selected from the group consisting of E1, E6, E7, E27, E31, E33, E39, E49, E56, E58, E63, and E65; and (d) an —(CH₂)ₘ₁—(C═C)_{q}—(CH₂)ₘ₂—CH₃ moiety selected from the group consisting of Sc12, Sc14, Sc16, and Sc18.

In particular aspects, R is:

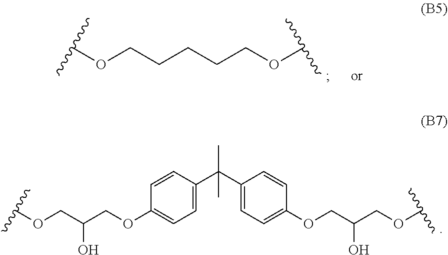 [SMILES: *OCCCCCO* ; *OCC(O)COc1ccc(C(C)(C)c2ccc(OCC(O)CO*)cc2)cc1]

In particular aspects, R′ is:

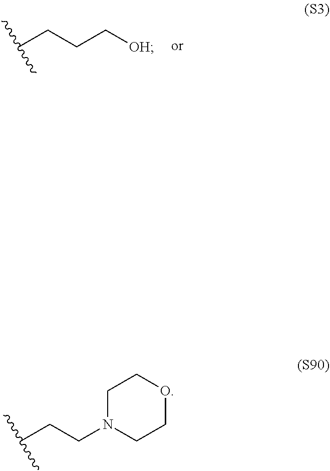 [SMILES: *CCCO ; "or" ; *CCN1CCOCC1]

In particular aspects, R″ is:

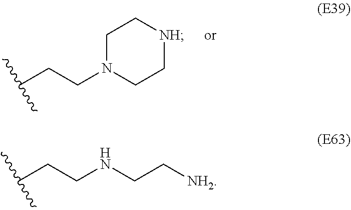 [SMILES: *CCN1CCNCC1 ; *CCNCCN]

In particular aspects, —(CH₂)ₘ₁—(C═C)_{q}—(CH₂)ₘ₂—CH₃ is Sc12.

In yet more particular aspects, the nanoparticle comprises B7-S90, Sc12-E63, 50%/50% ratio of S90/Sc16; or B5-S3, Sc12-E39, 70%/30% ratio of S3/Sc16.

In certain aspects, the compound of formula (I) comprises greater than 50% of the dry particle mass.

In some aspects, the nanoparticle further comprises one or more additional compounds selected from formula (I) and/or lipid-polyethylene glycol (PEG). In certain aspects, the lipid-PEG is selected from the group consisting of 1,2-dimyristoyl-rac-gly cero-3-methoxypolyethylene glycol 2000 (DMG-PEG2k) and C18-PEG2k. In particular aspects, the lipid-PEG comprises DMG-PEG2k.

In certain aspects, the nanoparticle comprises a mass percent of lipid PEG from about 2 wt % to about 10 wt %.

In certain aspects, the nanoparticle has a zeta-potential that varies with a weight percent of lipid-PEG, wherein the zeta-potential has a range selected from about −12 mV to about +18 mV or, in other aspects, from about −5 mV to about +5 mV.

In certain aspects, the nanoparticle comprises a plurality of nanoparticles having a polydispersity of less than about 0.2.

In certain aspects, the nanoparticle comprises at least three components selected from: (i) one or more of compounds of formula (I), (ii) one or more lipid-PEG, and (iii) one or more nucleic acids.

In certain aspects, the nanoparticle comprises about a 30:1 ratio of a compound of formula (I) to the one or more nucleic acids.

In some aspects, the one or more nucleic acids is selected from the group consisting of an oligonucleotide, a cyclic dinucleotide, plasmid DNA, linear DNA, siRNA, miRNA, and mRNA. In particular aspects, the one or more nucleic acids comprise an mRNA. In more particular aspects, the mRNA comprises self-amplifying mRNA (SAM). In certain aspects, the SAM comprises between about 15,000 to about 20,000 nucleotides. In particular aspects, the mRNA comprises a self-amplifying mRNA (SAM) construct encoding rabies virus glycoprotein.

In some aspects, the nanoparticle further comprises one or more excipients. In certain aspects, the one or more excipients include one or more cryoprotectants, one or more sugars or sugar alcohols, MgCl₂, and combinations thereof. In particular aspects, the one or more cryoprotectants comprise a sugar. In more particular aspects, the sugar is selected from the group consisting of glucose, fructose, sorbitol, mannitol, sucrose, trehalose, and raffinose. In particular aspects, the one or more sugar alcohols comprise sorbitol. In certain aspects, the nanoparticle is lyophilized. In certain aspects, the nanoparticle comprises a storable powder.

In some aspects, the presently disclosed subject matter provides a vaccine comprising a presently disclosed nanoparticle. In certain aspects, the vaccine comprises a vaccine against an infectious disease, including coronavirus, influenza, rabies, Ebola, dengue, polio, and hepatitis. In particular aspects, the vaccine comprises a rabies vaccine. In some aspects, the vaccine comprises a vaccine against cancer or against an autoimmune disease, including multiple sclerosis, type 1 diabetes, lupus, celiac disease, colitis, Crohn's disease, and rheumatoid arthritis.

In some aspects, the presently disclosed subject matter provides a method for delivering one or more nucleic acids to a subject, the method comprising administering a presently disclosed nanoparticle or vaccine to the subject. In certain aspects, the one or more nucleic acids is selected from the group consisting of an oligonucleotide, a cyclic dinucleotide, plasmid DNA, linear DNA, siRNA, miRNA, and mRNA. In particular aspects, the one or more nucleic acids comprise an mRNA. In more particular aspects, the mRNA comprises self-amplifying mRNA (SAM). In certain aspects, the SAM comprises between about 15,000 to about 20,000 nucleotides. In more particular aspects, the mRNA comprises a self-amplifying mRNA (SAM) construct encoding rabies virus glycoprotein. In certain aspects, the administering is selected from the group consisting of intramuscular, systemic, intranasal, intraocular, and intracranial. In particular aspects, the administering is intramuscular. In certain aspects, the SAM reaches a cytosol of a cell of the subject intact.

In some aspects the presently disclosed subject matter provides a method for treating or presenting a disease or condition, the method comprising administering a presently disclosed nanoparticle or vaccine to a subject in need of treatment thereof. In certain aspects, the one or more nucleic acids is selected from the group consisting of an oligonucleotide, a cyclic dinucleotide, plasmid DNA, linear DNA, siRNA, miRNA, and mRNA. In particular aspects, the one or more nucleic acids comprise an mRNA. In more particular aspects, the mRNA comprises self-amplifying mRNA (SAM). In certain aspects, the SAM comprises between about 15,000 to about 20,000 nucleotides. In yet more particular aspects, the mRNA comprises a self-amplifying mRNA (SAM) construct encoding rabies virus glycoprotein. In certain aspects, the administering is selected from the group consisting of intramuscular, systemic, intranasal, intraocular, and intracranial. In particular aspects, the administering is intramuscular. In more particular aspects, the disease or condition is rabies. In certain aspects, the subject is a human or an animal.

In some aspects, the presently disclosed subject matter provides a kit comprising one or more of: one or more compounds of formula (I), one or more self-amplifying mRNAs (SAMs), one or more lipid PEGs, one or more reagents, and instructions for use.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, FIG. 1D, FIG. 1E, and FIG. 1F are a schematic of SAM delivery via polymeric nanoparticles. (FIG. 1A) SAM structure including a 5′ cap, 5′ untranslated region (UTR), non-structural protein genes 1-4 from alphavirus, GOI, 3′ UTR, and PolyA tail. (FIG. 1B) Generalized structure of PBAE polymer, naming scheme for 4-component polymer and cartoon of assembled nanoparticle with PEG-lipid (FIG. 1C) DLS measurement of polymeric nanoparticles with and without SAM. (FIG. 1D) TEM microscopy of SAM nanoparticles. (FIG. 1E) Effect of PEG-lipid inclusion on NP diameter, polydispersity and zeta potential. (FIG. 1F) Physiochemical properties and encapsulation efficiency of the two lead nanoparticle structures (mean±SD of three measurements);

FIG. 2A, FIG. 2B, FIG. 2C, and FIG. 2D demonstrate the identification of nanoparticles effective for in vitro delivery of SAM to C2C12 murine myoblasts. (FIG. 2A) A library of 196 PBAEs were synthesized combinatorially from 28 base acrylate terminated polymers and 7 end-cap monomers. Polymers were screened in 384-well plates in C2C12 cells for transfection at a dose of 1 ng of eGFP SAM per well at two w/w ratios. Each cell of the heatmap shows mean of two wells of a 384-well plate. Transfection of myoblasts using eGFP SAM was strongly improved compared to 5mou eGFP mRNA used at the same dose as visible by (FIG. 2B) Microscopy (25 ng per well) and (FIG. 2C) Quantified percent transfection and (FIG. 2D) Selected nanoparticles for follow-up dose-titration screening were potent down to 20.6 pg/well in a 96-well format;

FIG. 3A, FIG. 3B, FIG. 3C, FIG. 3D, FIG. 3E, and FIG. 3F demonstrate the in vivo efficacy of intramuscular SAM delivery using luciferase SAM with expression assessed 10 days following injection. (FIG. 3A) Selection of polymer to SAM w/w ratio and (FIG. 3B) Particle mass fraction of DMG-PEG2k using nanoparticle 7-90, c12-63, 50% Sc12. (FIG. 3C) Violin plots of intramuscular luminescence measured at day 10 for nine PBAE NPs compared to naked SAM for a dose of 200 ng injected in 20 μL injection volume. N=10 IM injections per polymer; assessed using one-way ANOVA corrected for multiple comparisons to naked SAM injection. (FIG. 3D) Structures of lead polymers for intramuscular administration. (FIG. 3E) Representative IVIS images with the top two nanoparticle formulations compared to naked SAM. (FIG. 3F) Relationship between in vitro transfection of C2C12 cells in 96-well plates at a dose of 5 ng/well and in vivo luminescence following intramuscular administration at a dose of 200 ng/injection with 20 μL injection volume;

FIG. 4A and FIG. 4B demonstrate that polymeric nanoparticles delivering SAM enable immunogenic expression of antigen greater than naked SAM. (FIG. 4A) Schematic of FLuc-2A-rabies SAM dosing strategy (prime/boost). (FIG. 4B) Rabies Virus Neutralizing Antibody titers measured by RFFIT for top PBAE NP formulations 7-90, c12-63, 50% Sc12 and 5-3, c12-39, 30% Sc12 compared against naked SAM vaccinated and naive serum. N=10 animals per group; Mann-Whitney test for statistical significance against naked SAM injection;

FIG. 5A, FIG. 5B, FIG. 5C, FIG. 5D, and FIG. 5E illustrate PBAE Synthesis. (FIG. 5A) Generalized synthesis of PBAEs in DMF followed by polymer end-capping with small molecule amine endcaps to yield linear, amphiphilic PBAEs. (FIG. 5B) Selected diacrylate monomers. (FIG. 5C) Selected hydrophilic amine monomers. (FIG. 5D) Selected hydrophobic amine monomers. (FIG. 5E) Selected amine end-cap monomers used in synthesis of the presented PBAEs;

FIG. 6A and FIG. 6B demonstrate that inclusion of PEG-lipid does not improve transfection in vitro. (FIG. 6A) Transfection efficiency of SAM nanoparticles encoding eGFP added to confluent monolayers of C2C12 cells with SAM nanoparticles encoding eGFP formed under three different conditions. Solid lines show results for NPs prepared in 100 v % aqueous MgAc₂ buffer with no DMG-PEG2k. Dashed lines show NPs prepared in 100 v % aqueous MgAc₂ buffer with 10 m % DMG-PEG2k. Dotted lines show NPs prepared in 50 v % aqueous MgAc₂ buffer and 50 v % EtOH with 10 m % DMG-PEG2k followed by dialysis for 75 minutes into 150 mM PBS, pH 7.4. Inclusion of DMG-PEG2k reduced transfection efficiency in vitro, which was partially rescued in some formulations by dialysis. Points show ±SD of four wells of a 96 well plate. (FIG. 6B) Representative microscope images of transfected wells of C2C12 myoblasts showing reduction in efficiency of transfection of PEGylated formulations for polymer 7-4, c12-63, 50% Sc12 in vitro at doses below 185 μg per well. Scale bars indicate 1 μm;

FIG. 7A, FIG. 7B, and FIG. 7C show supporting data for in vitro screening of PBAE polymers. (FIG. 7A) In vitro transfection relative cell viability assessed by nuclei counting normalized to untreated wells. Each well represents the mean of two wells of a 384-well plate. (FIG. 7B) Additional minimally effective polymers screened for SAM delivery in 384-well plates for transfection efficiency using eGFP SAM at a dose of 1 ng/well relative to untreated cells and (FIG. 7C) Cellular viability of these polymers as assessed by nuclei counting relative to untreated cells;

FIG. 8A and FIG. 8B demonstrate alkyl-side chain hydrophobicity influence on in vitro transfection of SAM to differentiated C2C12 myoblasts. Experiments were performed using polymer 7-90, Scn-63 with alkyl side-chains of length n and m % mole fraction alkyl side-chain monomer. All nanoparticles were prepared with GFP SAM at 30 w/w ratio without DMG-PEG2k. (FIG. 8A) Increasing the alkyl-amine side-chain length while maintaining mole fraction of alkyl side-chain at 30% demonstrated that more hydrophobic alkyl-side chains increased efficacy of transfection in vitro. (FIG. 8B) Increasing the alkyl-amine side-chain mole fraction while keeping the alkyl length constant at 12 carbons similarly increased transfection in vitro;

FIG. 9A, FIG. 9B, FIG. 9C, FIG. 9D, FIG. 9E, FIG. 9F, and FIG. 9G show selection of murine intramuscular dose and timepoint for luminescence measurements. (FIG. 9A) Identification of peak expression of SAM following intramuscular injection between 10-21 days with a dose of 2 μg of SAM packaged in 7-90, c12-63, 50% Sc12 nanoparticles formulated with 10 mass % DMG-PEG2k. (FIG. 9B) Intramuscular injection volume and dose titration in mice of naked SAM and PBAE nanoparticles. (FIG. 9C) Injection doses and volumes used for SARS-CoV-2 mRNA vaccines used clinically. (FIG. 9D) Injection dose, volume, SAM concentration and fold luminescence over naked SAM achieved using PBAE nanoparticles. (FIG. 9E) Body surface area scaling between mice and adult humans results in a scaling factor of 228 using a lower end body surface area of 1.6 m² for an adult human female. Using a surface area of 1.9 m² for an adult human male would result in a higher scaling factor. (FIG. 9F) Scaling intramuscular injection volumes by body surface area results in injection volumes greater than acceptable for human intramuscular injection volumes (red), where 0.5 mL is considered a maximum acceptable injection volume. (FIG. 9G) Calculations for scaling intramuscular mRNA doses from those evaluated in mice (0.05 μg to 7.5 μg) to an equivalent surface area scaled dose for administration in humans. Given the dosage constraints, a 0.2 μg dose (green) was selected for evaluation of intramuscular administration in mice; and

FIG. 10 shows expression of self-amplifying mRNA compared against 5mou mRNA in vitro in C2C12 cells, demonstrating the enormous increase in overall expression when using SAM. For comparison, branched polyethylenimine (BPEI) NPs at a 2:1 w/w ratio formed with SAM were dosed at the same dose of 50 ng of nucleic acid per well. BPEI is a gold standard transfection agent used commercially. The comparison here demonstrates the usefulness of the new polymers, such as 7-90, c12-63, disclosed herein. BPEI is demonstrated to be ineffective as a standard cationic polymer to transfect these difficult to transfect cells. The advantage of making formulations with these polymers and SAM also is highlighted by these studies. While an assortment of nucleic acids may be used with this technology (including plasmid and linear DNA, siRNA, miRNA, mRNA, and other nucleic acids), SAM is especially effective as a cargo, when mixed with the polymer to form a nanoparticle.

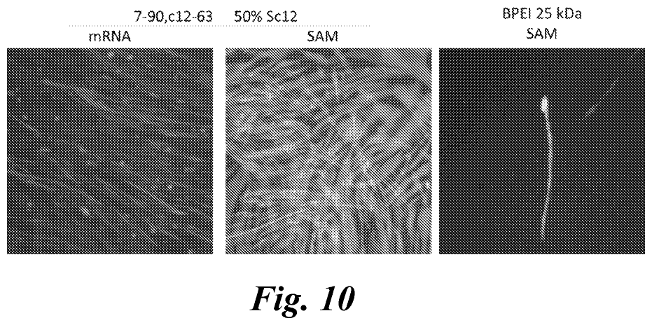

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Polymers and Nanoparticles for Intramuscular Nucleic Acid Delivery

The presently disclosed subject matter provides formulations of nanoparticles and their use for mRNA delivery. In particular embodiments, the delivery of the mRNA is via intramuscular (IM) injection of the nanoparticle formulations. Historically, conventional poly(beta-amino esters) (PBAEs) are poor vehicles for IM delivery. Previous, non-viral systems for mRNA delivery have generally all been lipid-based/liposome-based systems. It was not clear, however, based on the current state of the art at the time of this filing, that biodegradable polymers could achieve high levels of mRNA delivery, especially to muscle.

To overcome the difficulties associated with the use of PBAEs for IM delivery, the presently disclosed subject matter describes, in part, an empirical study exploring and testing a large library of materials to elucidate nanoparticle formulations that work well for IM injection. The exploring and testing of the large library of materials was accomplished through high throughput synthesis and screening of PBAEs. For example, as provided in more detail herein below, some polymer structures that were expected to work did not (for example, 5-90, c12-63 does not work well for IM injection), whereas other polymer structures did, e.g., 5-3, c12-63.

For example, these empirical studies indicated that canonical linear PBAEs that lack alkyl side-chains do not work well either in vitro or in vivo, even when formulated with PEG-lipid. Further, these studies indicated that PEGylation via admixing with a PEG-lipid, such as DMG-PEG2k or other lipid PEGS, appears to be required for in vivo efficacy. Without wishing to be bound to any one particular theory, it is thought that this efficacy is facilitated by modulating the zeta potential of lipid-PEG/PBAE nanoparticles. In contrast, addition of a lipid-PEG, e.g., DMG-PEG2k, is not required for in vitro administration and the addition of lipid-PEG to the presently disclosed nanoparticle formulations actually reduces transfection efficacy.

### A. Compositions

Accordingly, in some embodiments, the presently disclosed subject matter provides a biodegradable cationic polyester for intramuscular delivery of nucleic acids, including self-amplifying mRNA.

As used herein, “biodegradable” polymers and/or nanoparticles are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). Such components preferably do not induce inflammation or other adverse effects in vivo. In some instances, the chemical reactions relied upon to break down the biodegradable compounds are uncatalyzed.

In certain embodiments, the biodegradable polymers and/or nanoparticles comprise a chemical moiety having one or more degradable linkages, such as an ester linkage, a disulfide linkage, an amide linkage, an anhydride linkage, and a linkage susceptible to enzymatic degradation. Representative degradable linkages include, but are not limited to:

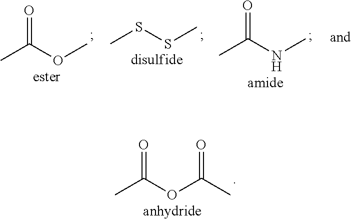

In some embodiments, the biodegradable polymer and/or nanoparticle comprises a poly(beta-amino ester) (PBAE). Exemplary PBAEs suitable for use with the presently disclosed subject matter include those disclosed in:
U.S. Pat. No. 9,884,118 for Multicomponent Degradable Cationic Polymers, to Green et al., issued Feb. 6, 2018;
U.S. Pat. No. 9,802,984 for Biomimetic Peptide and Biodegradable Delivery Platform for the Treatment of Angiogenesis- and Lymphangiogenesis-Dependent Diseases, to Popel et al., issued Oct. 31, 2017;
U.S. Pat. No. 9,717,694 for Peptide/Particle Delivery Systems, to Green et al., issued Aug. 1, 2017;
U.S. Pat. No. 8,992,991 for Multicomponent Degradable Cationic Polymers, to Green et al., issued Mar. 31, 2015;
U.S. Patent Application Publication No. 20180256745 for Biomimetic Artificial Cells: Anisotropic Supported Lipid Bilayers on Biodegradable Micro and Nanoparticles for Spatially Dynamic Surface Biomolecule Presentation, to Meyer et al., published Sep. 13, 2018;
U.S. Patent Application Publication No. 20180112038 for Poly(Beta-Amino Ester)-Co-Polyethylene Glycol (PEG-PBAE-PEG) Polymers for Gene and Drug Delivery, to Green et al., published Apr. 26, 2018;
U.S. Patent Application Publication No. 20170216363 for Nanoparticle Modification of Human Adipose-Derived Mesenchymal Stem Cells for Treating Brain Cancer and other Neurological Diseases, to Quinones-Hinojosa and Green, published Aug. 3, 2017;
U.S. Patent Application Publication No. 20150273071 for Bioreducible Poly (Beta-Amino Ester)s For siRNA Delivery, to Green et al., published Oct. 1, 2015;
U.S. Pat. No. 8,287,849 for Biodegradable Poly(beta-amino esters) and Uses Thereof, to Langer, et al., issued Oct. 16, 2012;
International PCT Patent Application Publication No. WO2020198145 for Gene Delivery Particles to Induce Tumor-Derived Antigen Presenting Cells, to Green, published Oct. 1, 2020;
each of which is incorporated by reference in their entirety.

Generally, the presently disclosed multicomponent degradable cationic polymers include a backbone derived from a diacrylate monomer (designated herein below as “B”), an amino-alcohol hydrophilic side-chain monomer (designated herein below as “S”), a hydrophobic side-chain monomer, and an amine-containing endcapping monomer (designated herein below as “E”). The endcapping group structures are distinct and separate from the polymer backbone structures and the side chain structures of the intermediate precursor molecule for a given polymeric material.

The presently disclosed PBAE compositions can be designated, for example, as B5-S4-E7 or 547, in which R is B5, R′ is S4, and R″ is E7, and the like, where B is the backbone and S is the side chain, followed by the number of carbons in their hydrocarbon chain, e.g., S4 comprises 4 alkylene groups. Endcapping monomers, E, are sequentially numbered according to similarities in their amine structures. Further, in some embodiments, the presently disclosed PBAE includes a hydrophobic side-chain, which is designated SC-XX, with XX being the number of carbon atoms in the chain.

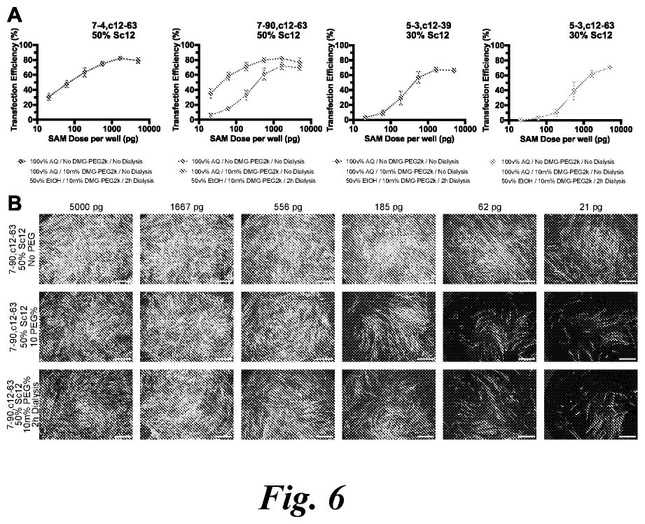

As shown in the reaction scheme provided in FIG. 6, acrylate monomers can be condensed with amine-containing side chain monomers. In some embodiments, the side chain monomers comprise a primary amine, but, in other embodiments, comprise secondary and tertiary amines. Side chain monomers may further comprise a C₁ to C₈ linear or branched alkylene, which is optionally substituted. Illustrative substituents include hydroxyl, alkyl, alkenyl, thiol, amine, carbonyl, and halogen.

Table 1 and Table 2 present, in more detail, particular monomers used for PBAE library synthesis. Acrylate terminated polymers were synthesized from small molecule diacrylate and primary amine monomers followed by high-throughput endcapping with 37 monomers organized into different structural categories. In certain embodiments, the linear and/or branched PBAE polymer has a molecular weight of from 5 to 10 kDa, or a molecular weight of from 10 to 15 kDa, or a molecular weight of from 15 to 25 kDa, or a molecular weight of from 25 to 50 kDa.

Accordingly, in some embodiments, the presently disclosed subject matter provides a nanoparticle comprising a compound of formula (I) and one or more nucleic acids:

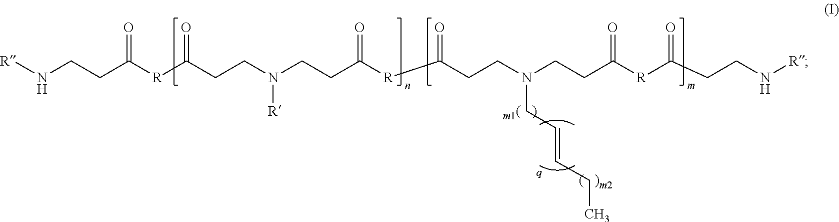

wherein: m and n are each independently an integer from 1 to 10,000; m1 is an integer selected from 0, 1, 2, 3, 4, 5, 6, 7, 8, 9, and 10; m2 is an integer selected from the group consisting of 1, 2, 3, 4, 5, 6, 7, 8, 9, 10, 11, 12, 13, 14, 15, 16, 17, 18, 19, and 20; q is an integer selected from 0 or 1; wherein —(CH₂)ₘ₁—(C═C)_{q}—(CH₂)ₘ₂—CH₃ comprises a hydrophobic sidechain; R comprises a divalent radical comprising a biodegradable ester linkage and/or a bioreducible disulfide linkage; R′ is hydrophilic sidechain comprising a monovalent radical derived from a hydrophilic amine monomer; R″ is monovalent radical derived from an amine-containing end capping group; and pharmaceutically acceptable salts thereof.

In certain embodiments, R is selected from the group consisting of:

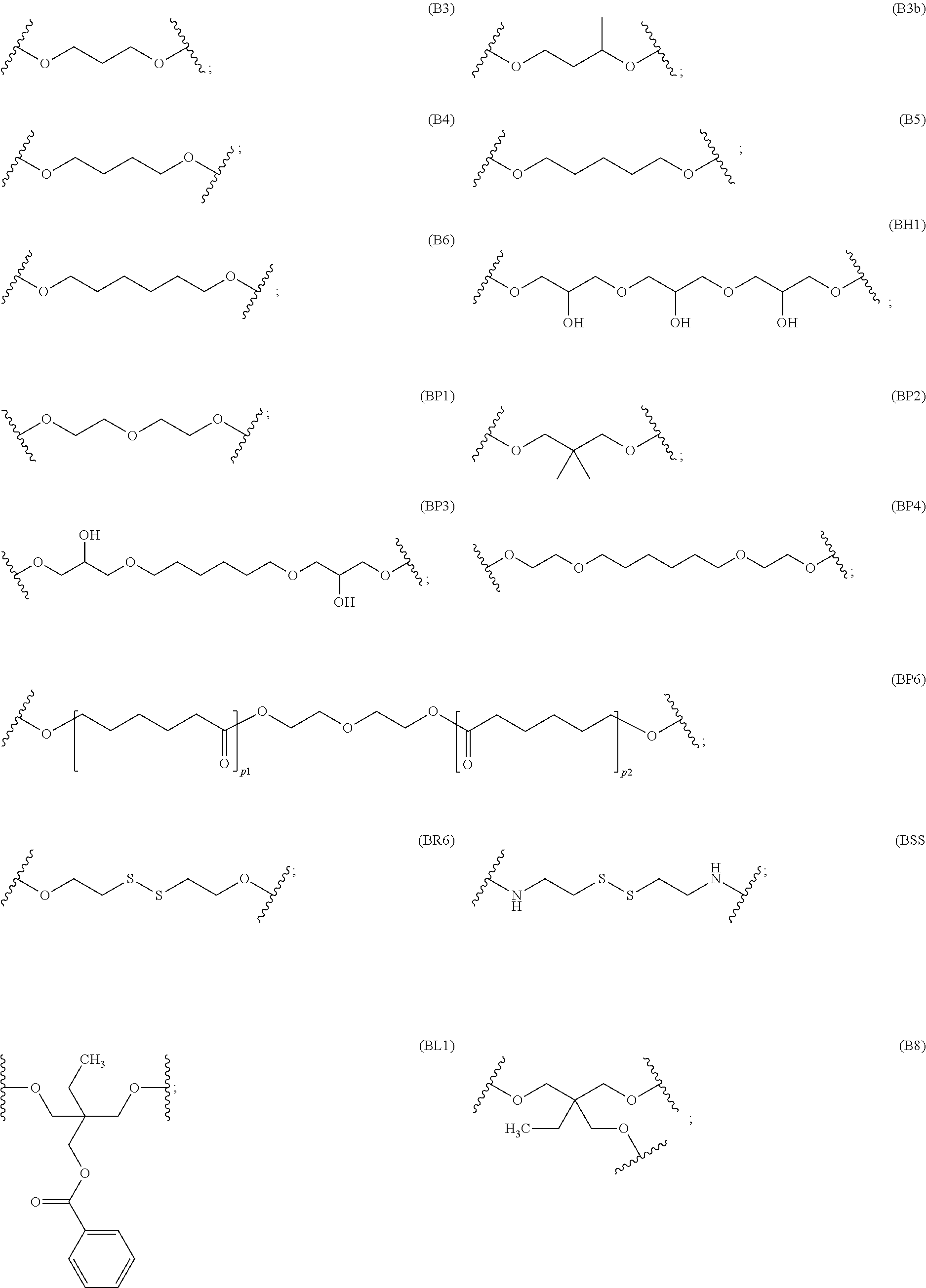

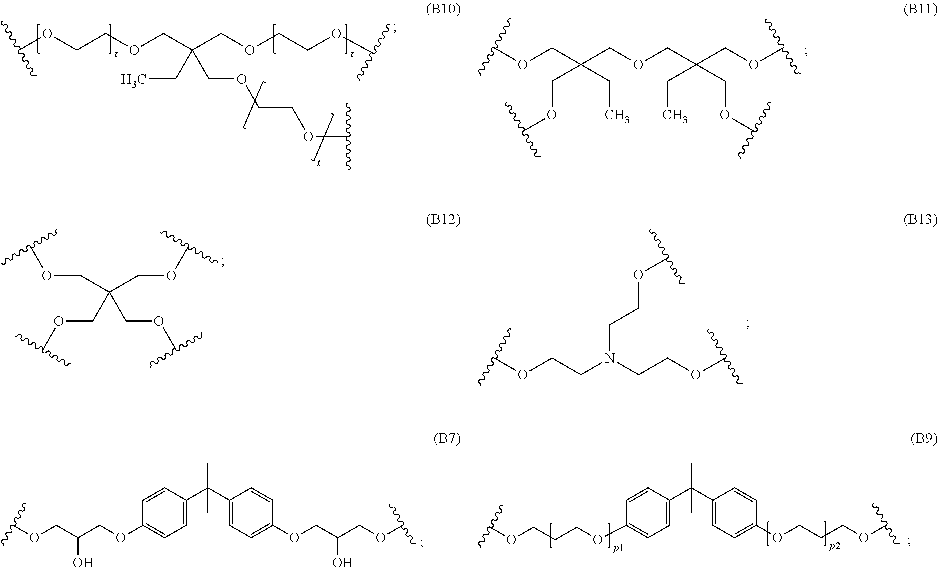

wherein each p1, p2, and t is independently an integer selected from the group consisting of 1, 2, 3, 4, 5, 6, 7, 8, 9, and 10.

In certain embodiments, R′ is selected from the group consisting of:

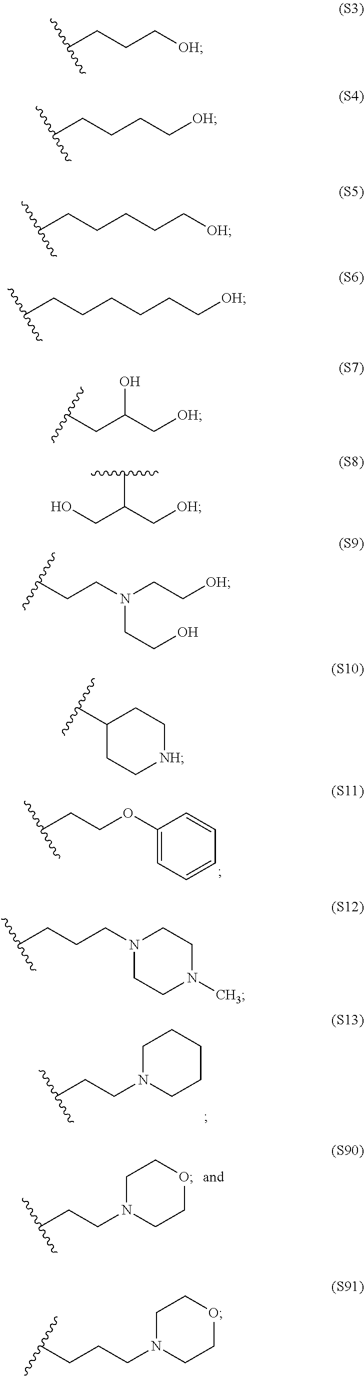

In certain embodiments, R″ is selected from the group consisting of:

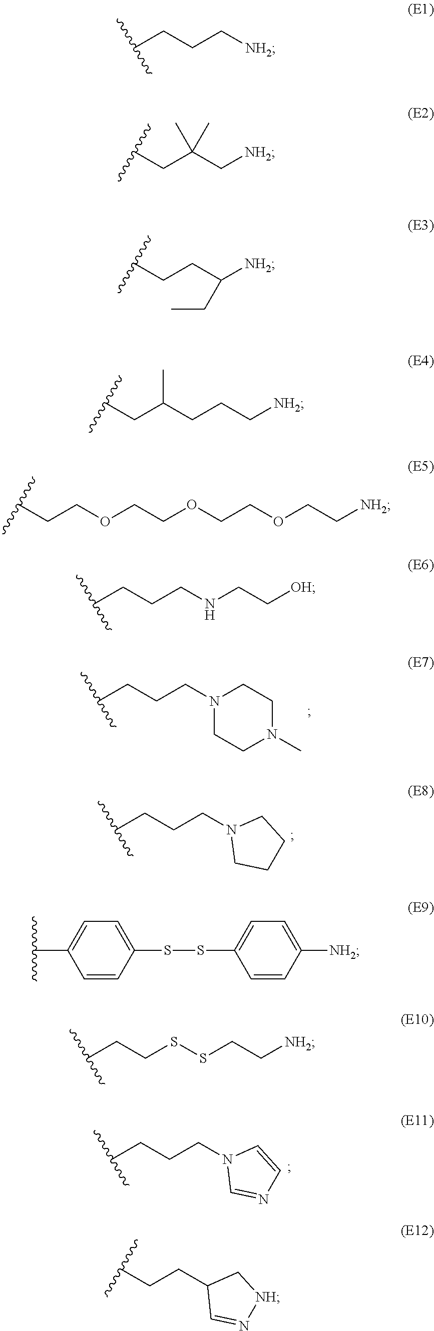

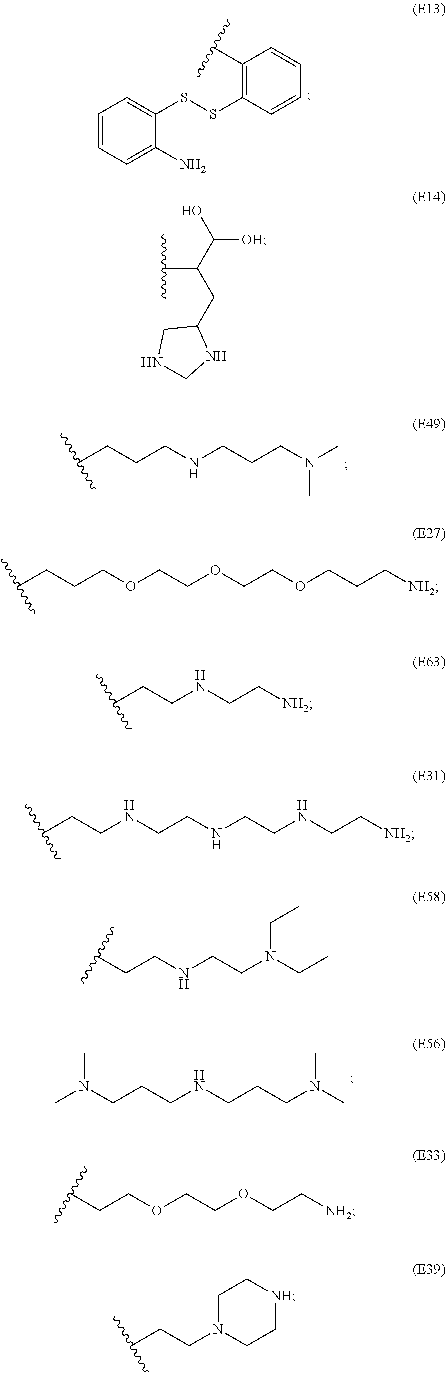

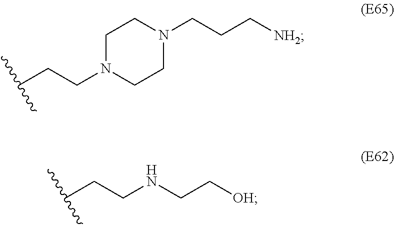 [SMILES: *CCN1CCN(CCCN)CC1 ; *CCNCCO]

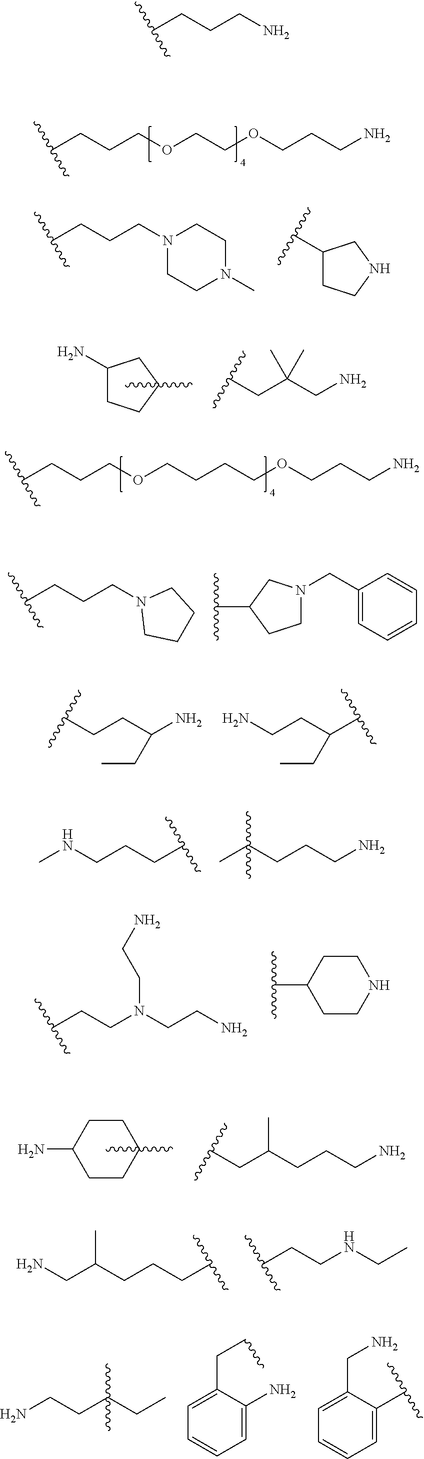

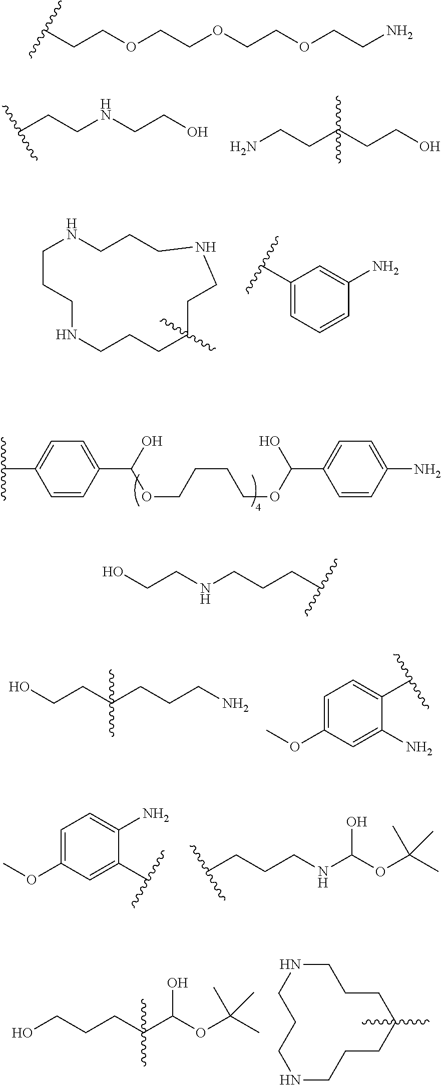

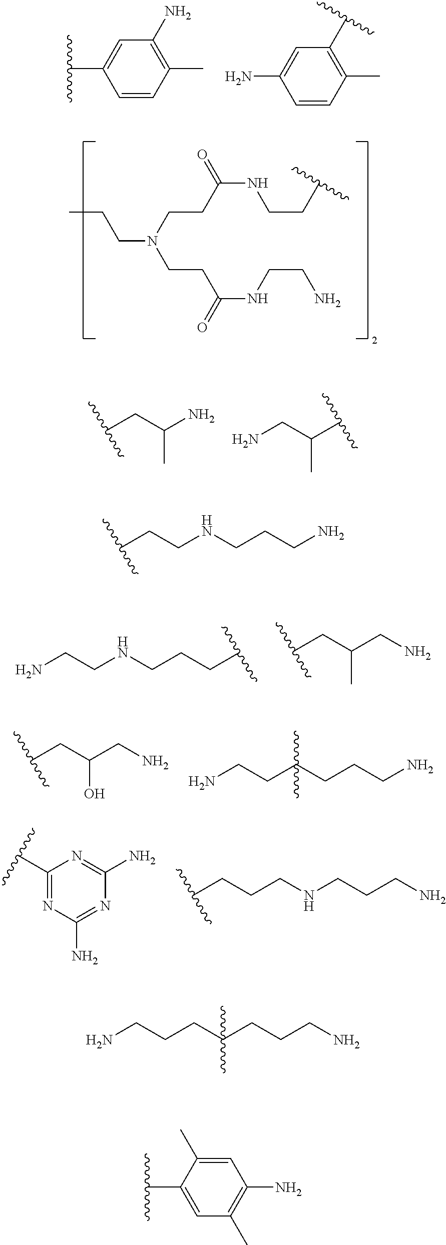

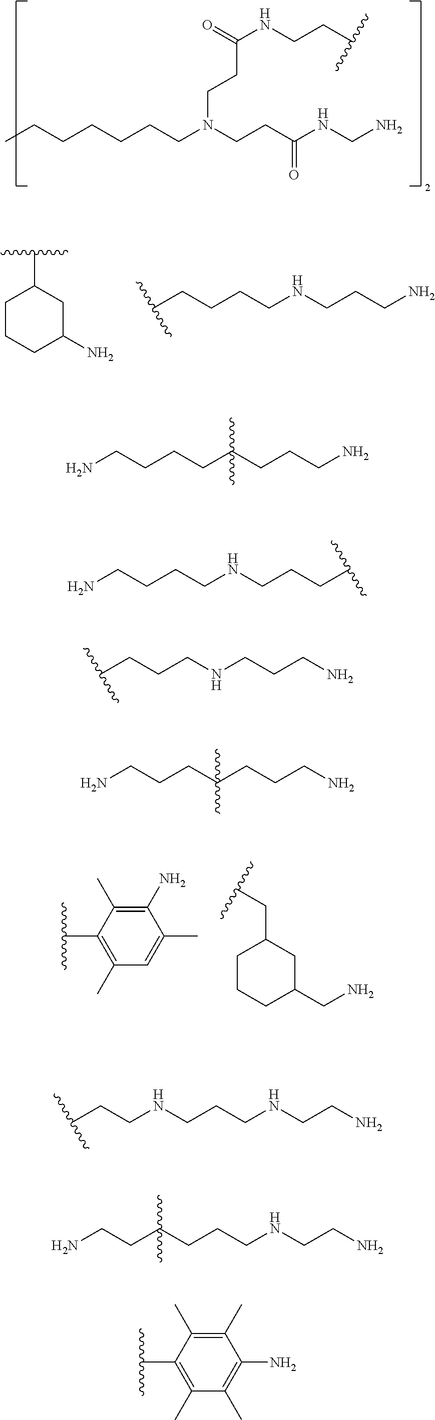

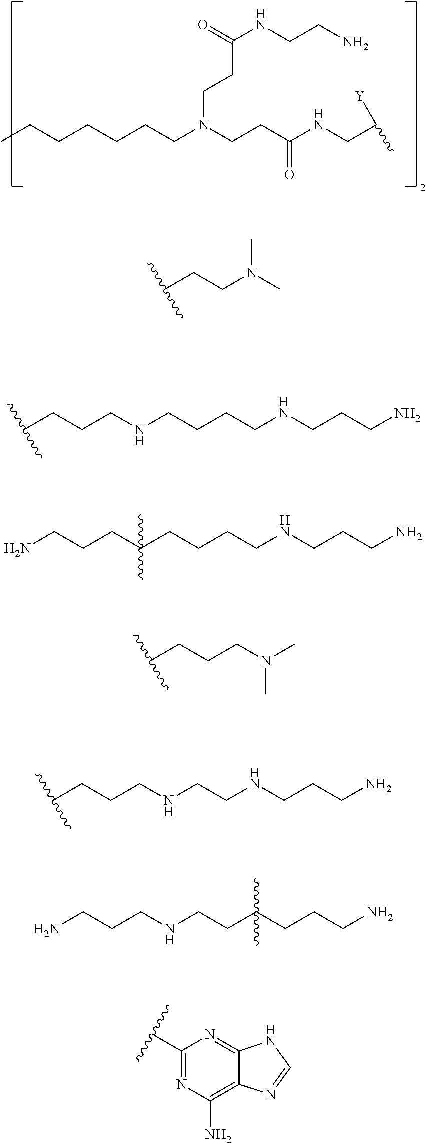

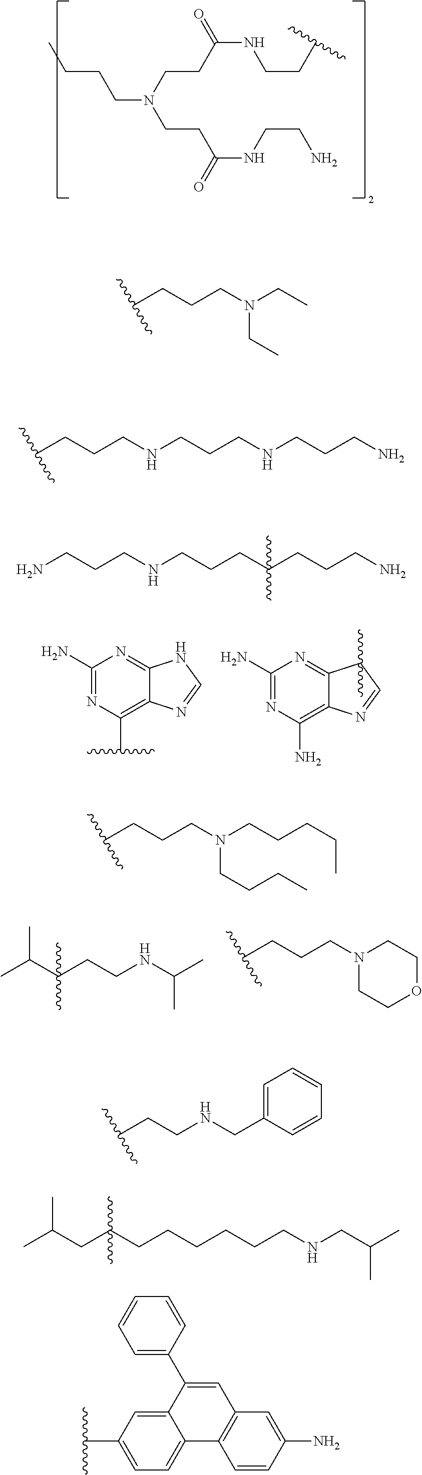

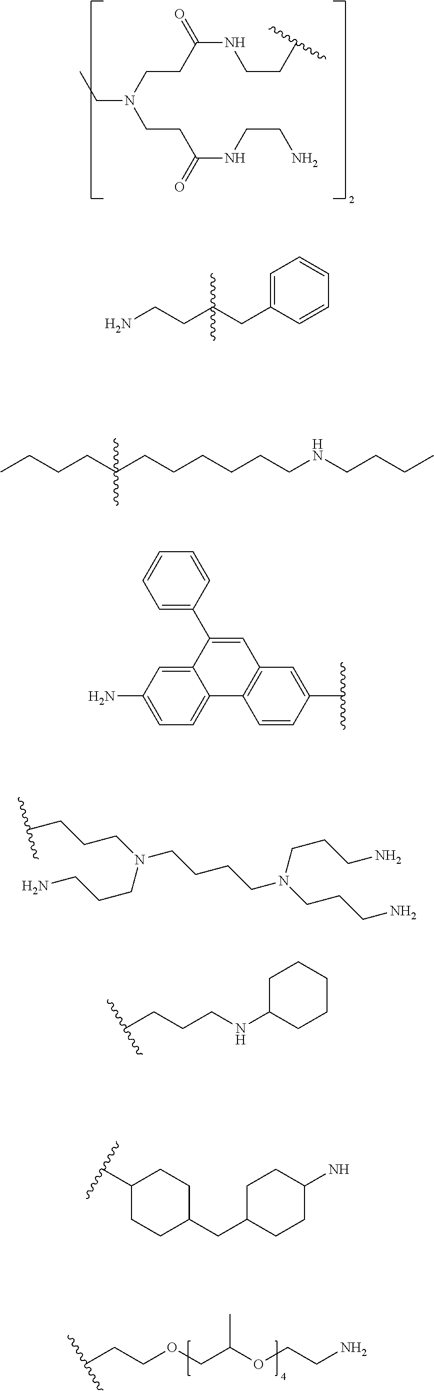

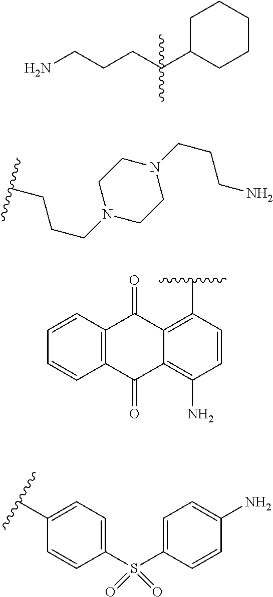

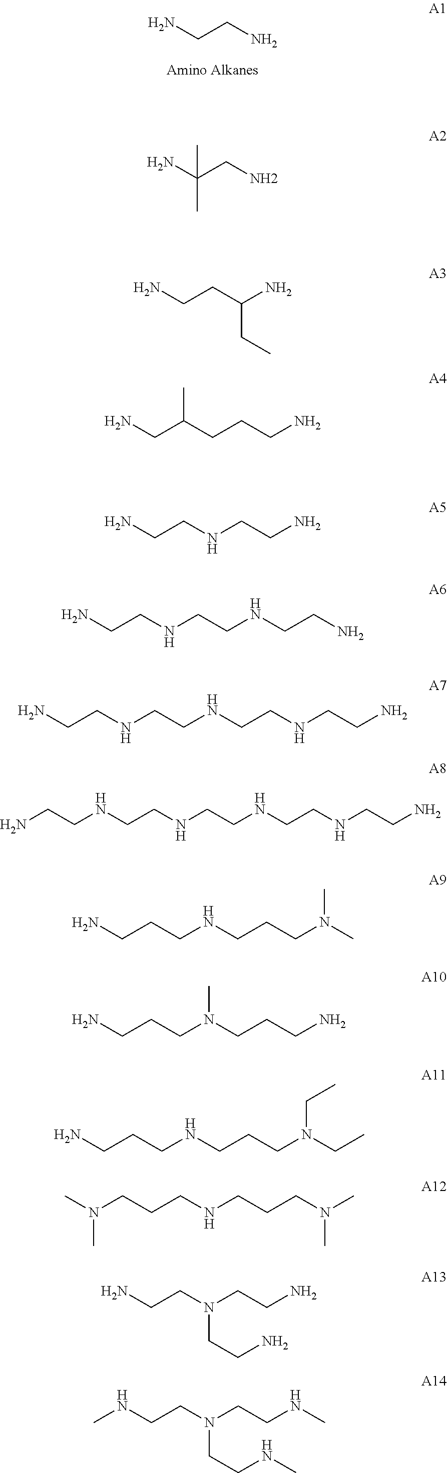

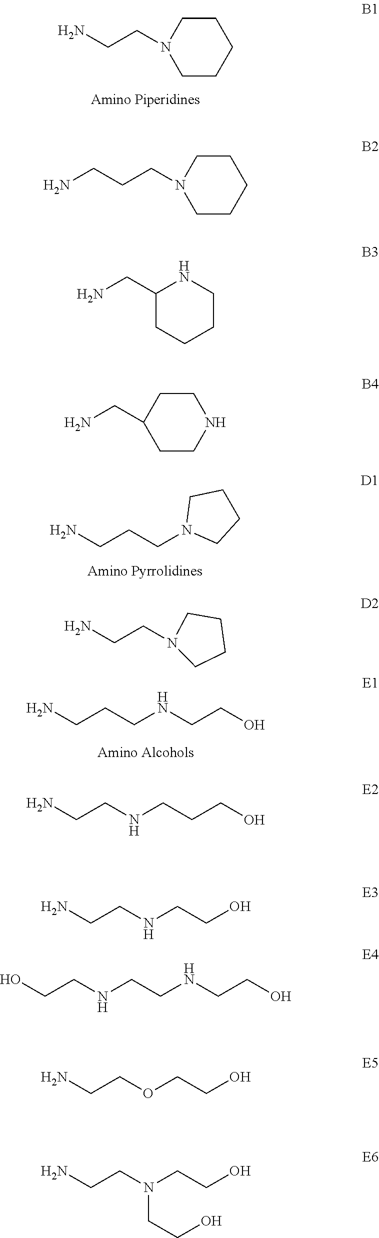

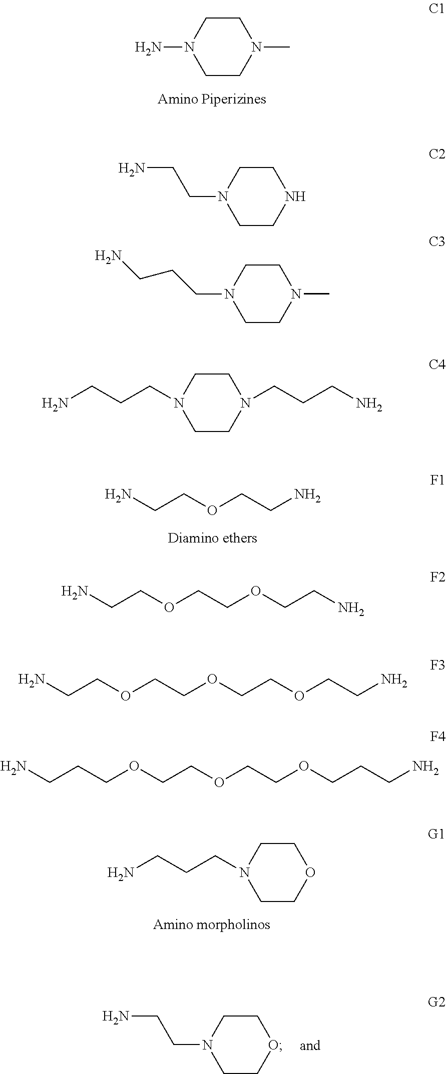

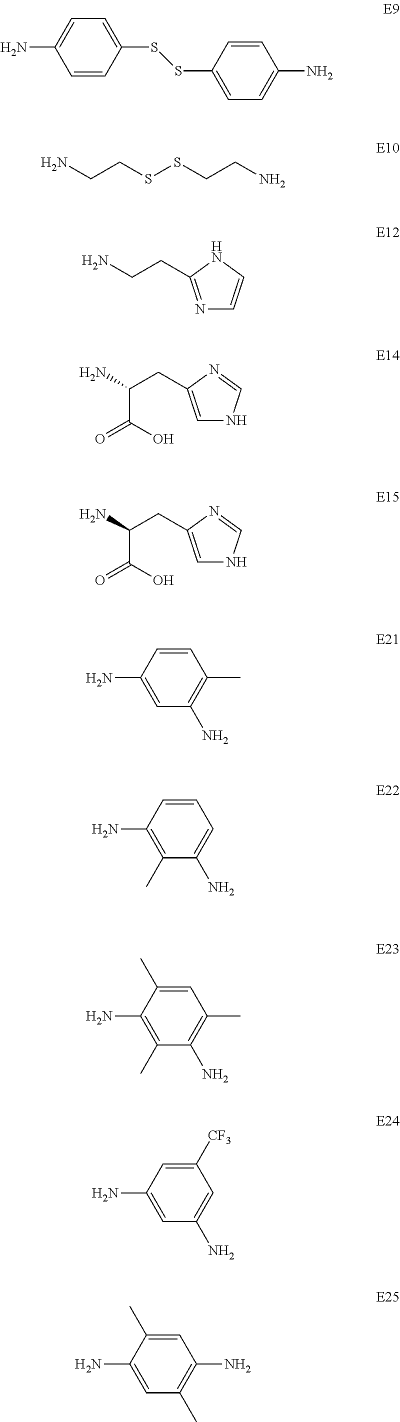

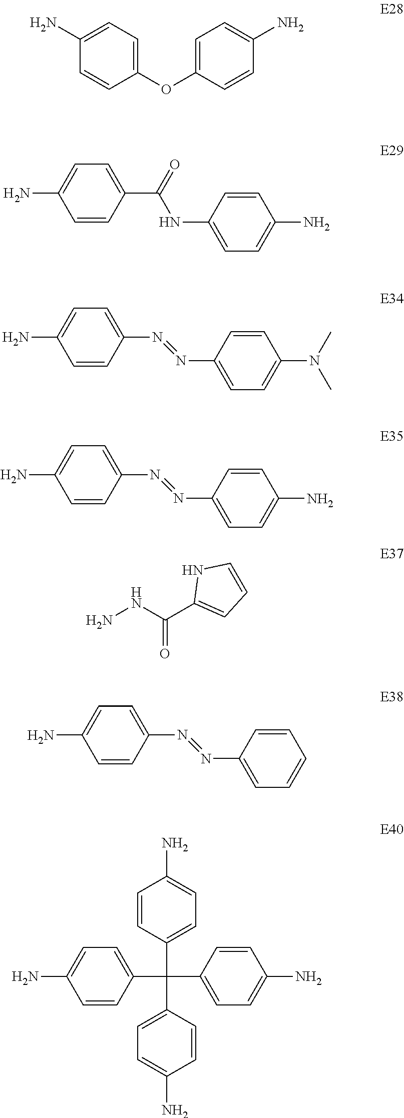

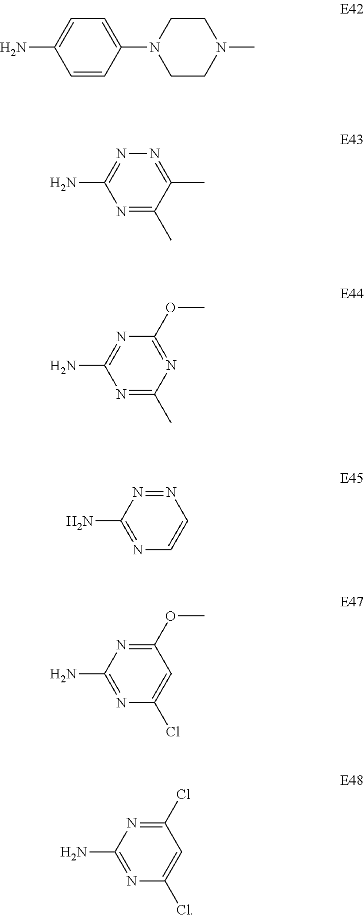

In certain embodiments, —(CH₂)ₘ₁—(C═C)_{q}—(CH₂)ₘ₂—CH₃ is selected from the group consisting of:

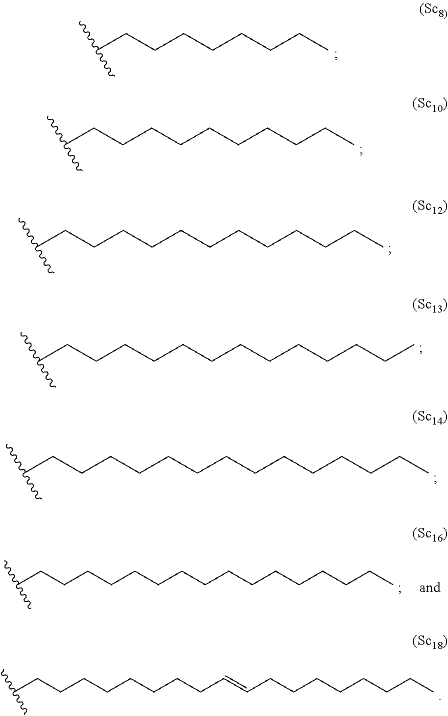

In particular embodiments, R is selected from the group consisting of:

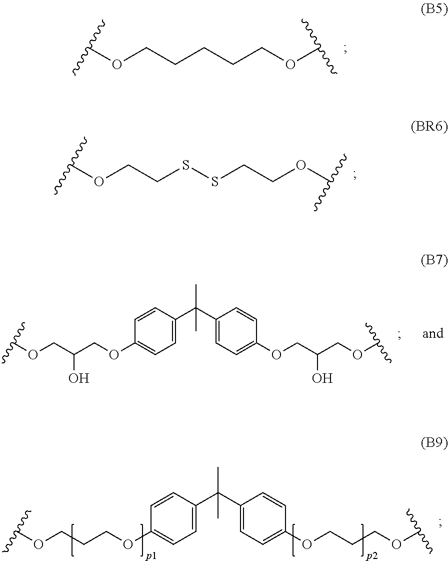

wherein each p1, p2, and t is independently an integer selected from the group consisting of 1, 2, 3, 4, 5, 6, 7, 8, 9, and 10.

In particular embodiments, R′ is selected from the group consisting of:

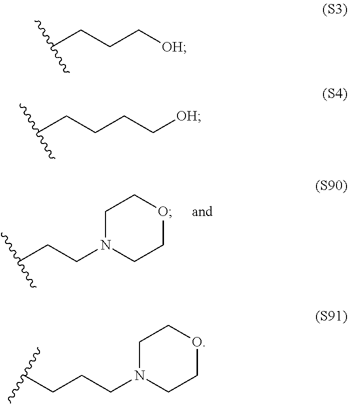 [SMILES: *CCCO ; *CCCCO ; *CCN1CCOCC1 ; *CCCN1CCOCC1]

In particular embodiments, R″ is selected from the group consisting of:

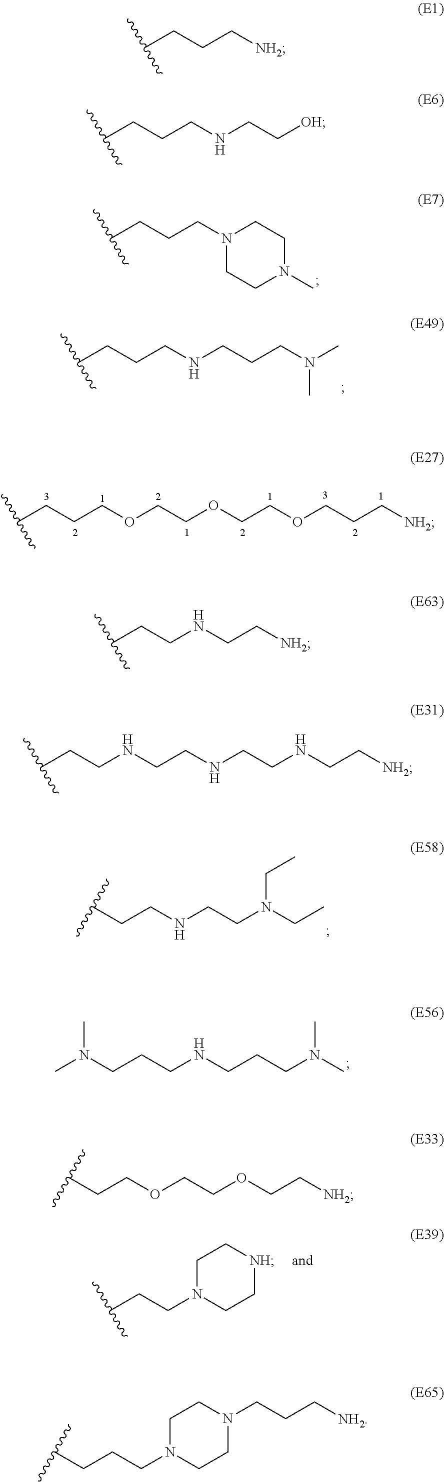

In particular embodiments, —(CH₂)ₘ₁—(C═C)_{q}—(CH₂)ₘ₂—CH₃ is selected from the group consisting of:

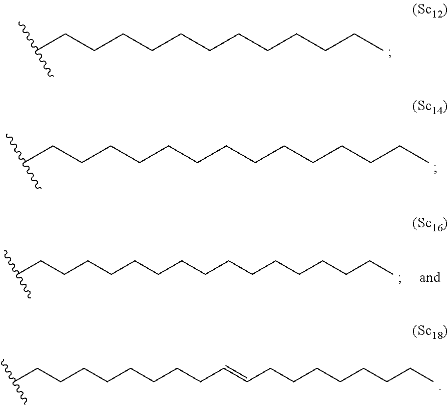 [SMILES: *CCCCCCCCCCCC ; *CCCCCCCCCCCCCC ; *CCCCCCCCCCCCCCCC ; *CCCCCCCC/C=C/CCCCCCCC]

In more particular embodiments, the compound of formula (I) comprises a combination of: (a) an R group selected from the group consisting of B5, B7, B9, and BR6; (b) an R′ group selected from the group consisting of S3, S4, S90, and S91; (c) an R″ group selected from the group consisting of E1, E6, E7, E27, E31, E33, E39, E49, E56, E58, E63, and E65; and (d) an —(CH₂)ₘ₁—(C═C)_{q}—(CH₂)ₙ₂—CH₃ moiety selected from the group consisting of Sc12, Sc14, Sc16, and Sc18.

In particular embodiments, R is:

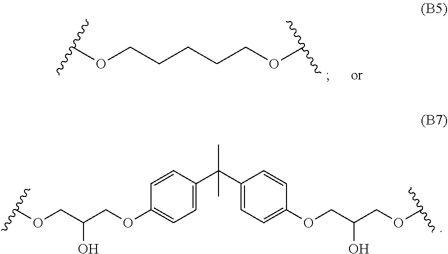 [SMILES: *OCCCCCO* ; *OCC(O)COc1ccc(C(C)(C)c2ccc(OCC(O)CO*)cc2)cc1]

In particular embodiments, is:

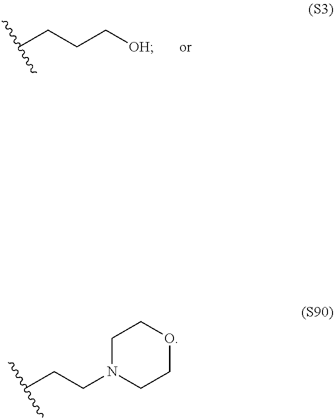 [SMILES: *CCCO ; *CCN1CCOCC1]

In particular embodiments, R″ is:

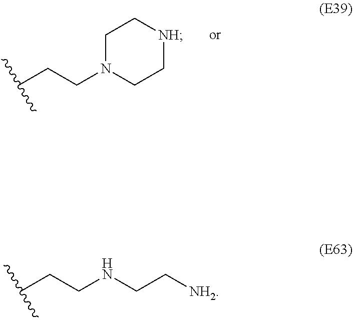 [SMILES: *CCN1CCNCC1 ; *CCNCCN]

In particular embodiments, —(CH₂)ₘ₁—(C═C)_{q}—(CH₂)ₘ₂—CH₃ is Sc12.

In yet more particular embodiments, the nanoparticle comprises B7-S90, Sc12-E63, 50%/50% ratio of S90/Sc16; or B5-S3, Sc12-E39, 70%/30% ratio of S3/Sc16.

In certain embodiments, the compound of formula (I) comprises greater than 50% of the dry particle mass.

In some embodiments, the nanoparticle further comprises one or more additional compounds selected from formula (I) and/or lipid-polyethylene glycol (PEG). In certain embodiments, the lipid-PEG is selected from the group consisting of 1,2-dimyristoyl-rac-gly cero-3-methoxypolyethylene glycol 2000 (DMG-PEG2k) and C18-PEG2k. In particular embodiments, the lipid-PEG comprises DMG-PEG2k.

In certain embodiments, the nanoparticle comprises a mass percent of lipid PEG from about 2 wt % to about 10 wt %, including about 2, 3, 4, 5, 6, 7, 8, 9, and 10 wt %.

In certain embodiments, a zeta-potential of the nanoparticle varies with a weight percent of lipid-PEG, wherein the zeta-potential has a range of −12 mV to +18 mV, including about −12, −11, −10, −9, −8, −7, −6, −5, −4, −3, −2, −1, 0, +1, +2, +3, +4, +5, +6, +7, +8, +9, +10, +11, +12, +13, +14, +15, +16, +17, and +18 mV, and in some embodiments between −5 mV to +5 mV, including about −5, −4, −3, −2, −1, 0, +1, +2, +3, +4, and +5 mV. In some embodiments, the zeta-potential is measured under an aqueous condition, for example, in 150 mM Phosphate Buffered Saline (PBS).

In certain embodiments, the nanoparticle comprises a plurality of nanoparticles having a polydispersity of less than about 0.2, including about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, and 0.20.

In certain embodiments, the nanoparticle comprises at least three components selected from: (i) one or more of compounds of formula (I), (ii) one or more lipid-PEG, and (iii) one or more nucleic acids.

In certain embodiments, the nanoparticle comprises about a 30:1 ratio of a compound of formula (I) to the one or more nucleic acids.

In some embodiments, the one or more nucleic acids is selected from the group consisting of an oligonucleotide, a cyclic dinucleotide, plasmid DNA, linear DNA, siRNA, miRNA, and mRNA. In particular embodiments, the one or more nucleic acids comprise an mRNA. In more particular embodiments, the mRNA comprises self-amplifying mRNA (SAM). In certain embodiments, the SAM comprises between about 15,000 to about 20,000 nucleotides. In particular embodiments, the mRNA comprises a self-amplifying mRNA (SAM) construct encoding rabies virus glycoprotein.

In some embodiments, the nanoparticle further comprises one or more excipients. In certain embodiments, the one or more excipients include one or more cryoprotectants, one or more sugars or sugar alcohols, MgCl₂, and combinations thereof. In particular embodiments, the one or more cryoprotectants comprise a sugar. In more particular embodiments, the sugar is selected from the group consisting of glucose, fructose, sorbitol, mannitol, sucrose, trehalose, and raffinose. In particular embodiments, the one or more sugar alcohols comprise sorbitol. In certain embodiments, the nanoparticle is lyophilized. In certain embodiments, the nanoparticle comprises a storable powder.

In some embodiments, the nanoparticle has at least one dimension in the range of about 50 nm to about 500 nm, or from about 50 to about 200 nm. Exemplary nanoparticles may have an average size (e.g., average diameter) of about 50, about 75, about 100, about 125, about 150, about 200, about 250, about 300, about 400 or about 500 nm. In some embodiments, the nanoparticle has an average diameter of from about 50 nm to about 500 nm, from about 50 nm to about 300 nm, or from about 50 nm to about 200 nm, or from about 50 nm to about 150 nm, or from about 70 to 100 nm. In embodiments, the nanoparticle has an average diameter of from about 200 nm to about 500 nm. In embodiments, the nanoparticle has at least one dimension, e.g., average diameter, of about 50 to about 100 nm. Nanoparticles are usually desirable for in vivo applications. For example, a nanoparticle of less than about 200 nm will better distribute to target tissues in vivo.

In certain embodiments, the presently disclosed subject matter provides a pharmaceutical formulation comprising one or more nucleic acids and a poly(beta-amino ester) (PBAE) of formula (I) in a pharmaceutically acceptable carrier.

As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art, and thus further examples and methods of incorporating each into compositions at effective levels need not be discussed here.

### B. Kits

In some embodiments, the presently disclosed subject matter provides a kit comprising one or more of: one or more compounds of formula (I), one or more self-amplifying mRNAs (SAMs), one or more lipid PEGs, one or more reagents, and instructions for use.

In certain embodiments, the disclosed kits comprise one or more containers, including, but not limited to a vial, tube, ampule, bottle and the like, for containing the pharmaceutical composition including one or more compounds of formula (I). The compounds of formula (I) may be solvated, in suspension, or powder form, and may then be reconstituted in the pharmaceutically acceptable carrier to provide the pharmaceutical composition. The one or more containers also can be carried within a suitable carrier, such as a box, carton, tube or the like. Such containers can be made of plastic, glass, laminated paper, metal foil, or other materials suitable for holding medicaments.

In certain embodiments, the container can hold a pharmaceutical composition and may have a sterile access port (for example the container may be an intravenous solution bag or a vial having a stopper pierceable by a hypodermic injection needle). Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### C. Vaccines

In some embodiments, the presently disclosed subject matter provides a vaccine comprising a presently disclosed nanoparticle. In certain embodiments, the vaccine comprises a vaccine against an infectious disease, including coronavirus, influenza, rabies, Ebola, dengue, polio, and hepatitis. In particular embodiments, the vaccine comprises a rabies vaccine. In some embodiments, the vaccine comprises a vaccine against cancer or against an autoimmune disease, including multiple sclerosis, type 1 diabetes, lupus, celiac disease, colitis, Crohn's disease, and rheumatoid arthritis.

### D. Methods for Delivering One or More Nucleic Acids to a Subject

In some embodiments, the presently disclosed subject matter provides a method for delivering one or more nucleic acids to a subject, the method comprising administering a presently disclosed nanoparticle or vaccine to the subject. In certain embodiments, the one or more nucleic acids is selected from the group consisting of an oligonucleotide, a cyclic dinucleotide, plasmid DNA, linear DNA, siRNA, miRNA, and mRNA. In particular embodiments, the one or more nucleic acids comprise an mRNA. In more particular embodiments, the mRNA comprises self-amplifying mRNA (SAM). In certain embodiments, the SAM comprises between about 15,000 to about 20,000 nucleotides. In more particular embodiments, the mRNA comprises a self-amplifying mRNA (SAM) construct encoding rabies virus glycoprotein. In certain embodiments, the administering is selected from the group consisting of intramuscular, systemic, intranasal, intraocular, and intracranial. In particular embodiments, the administering is intramuscular. In certain embodiments, the SAM reaches a cytosol of a cell of the subject intact.

### E. Methods of Treatment

In some embodiments the presently disclosed subject matter provides a method for treating or presenting a disease or condition, the method comprising administering a presently disclosed nanoparticle or vaccine to a subject in need of treatment thereof. In certain embodiments, the one or more nucleic acids is selected from the group consisting of an oligonucleotide, a cyclic dinucleotide, plasmid DNA, linear DNA, siRNA, miRNA, and mRNA. In particular embodiments, the one or more nucleic acids comprise an mRNA. In more particular embodiments, the mRNA comprises self-amplifying mRNA (SAM). In certain embodiments, the SAM comprises between about 15,000 to about 20,000 nucleotides. In yet more particular embodiments, the mRNA comprises a self-amplifying mRNA (SAM) construct encoding rabies virus glycoprotein. In certain embodiments, the administering is selected from the group consisting of intramuscular, systemic, intranasal, intraocular, and intracranial. In particular embodiments, the administering is intramuscular. In more particular embodiments, the disease or condition is rabies. In certain embodiments, the subject is a human or an animal.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed composition of formula (I), to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments±50%, in some embodiments±20%, in some embodiments±10%, in some embodiments±5%, in some embodiments±1%, in some embodiments ±0.5%, and in some embodiments±0.10% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Biodegradable Polyester Nanoparticle Vaccines Deliver Self-Amplifying mRNA in Mice at Low Doses

### 1.1 Overview

Delivery of self-amplifying mRNA (SAM) has high potential for infectious disease vaccination due its self-adjuvating and dose-sparing properties. Yet a challenge is the susceptibility of SAM to degradation and the need for SAM to reach the cytosol fully intact to enable self-amplification. Lipid nanoparticles have been successfully deployed at incredible speed for mRNA vaccination, but aspects such as cold storage, manufacturing, efficiency of delivery, and the therapeutic window would benefit from further improvement. To investigate alternatives to lipid nanoparticles, we developed a class of >200 biodegradable end-capped lipophilic poly(beta-amino ester)s (PBAEs) that enable efficient delivery of SAM in vitro and in vivo as assessed by measuring expression of SAM encoding reporter proteins. We evaluated the ability of these polymers to deliver SAM intramuscularly in mice, and identified a polymer-based formulation that yielded up to 37-fold higher intramuscular (IM) expression of SAM compared to injected naked SAM. Using the same nanoparticle formulation to deliver a SAM encoding rabies virus glycoprotein, the vaccine elicited superior immunogenicity compared to naked SAM delivery, leading to seroconversion in mice at RNA injection doses as low as 200 ng. These biodegradable nanomaterials may be useful in the development of next-generation RNA vaccines for infectious diseases.

### 1.2 Background

Certain end-capped poly(beta-amino ester) (PBAE) terpolymers have recently been developed for systemic delivery of mRNA, resulting in high efficacy of delivery to lung endothelial cells when administered intravenously in mice. Kaczmarek et al., 2018. Compared to PBAEs lacking alkyl-amine side chain monomers, these amphiphilic PBAEs enabled highly improved mRNA complexation, protection and delivery and further allowed co-complexation with PEG-lipids to reduce rapid reticuloendothelial system (RES) clearance of cationic nanoparticles. Karlsson et al., 2020. The alkyl chains of these PBAEs also enhanced structural stability of the nanoparticles by the hydrophobic effect, Kaczmarek et al., 2018, in contrast to canonical PBAE polymers which rely more on electrostatic interactions with nucleic acids to drive nanoparticle nucleation. Anderson et al., 2003. These features of amphiphilic PBAE terpolymers mimic much of what makes lipid nanoparticles highly effective for nucleic acid delivery, while also incorporating the primary benefits of PBAE polymers, including increased avidity compared to ionizable lipids, rapid ester degradation catalyzed by the tertiary amines in the backbone of the polymer, Wilson et al., 2019; Sunshine et al., 2011, and structural end-cap monomer diversity enabling differential cell targeting. Kim et al., 2014; Rui et al., 2022; Shmueli et al., 2012. Recent data has also highlighted that PBAE nanoparticles can be an order of magnitude more effective at endosomal escape to the cytosol compared to leading commercially available polymer and lipid-based transfection agents. Rui et al., 2022.

Due to the self-amplifying nature of SAM in the cytosol, modified nucleosides replacement cannot be used to generate viable SAM with improved stability, Erasmus et al., 2020, and requires intact delivery of full-length SAM molecules to the cytosol for activity; Blakney et al., 2021; Brito et al., 2015, these features coupled with the long transcript length of SAM makes SAM especially susceptible to degradation by extracellular nucleases compared to other nucleic acid cargos. Thus, SAM vaccines or therapeutics can directly benefit by encapsulation into nanoparticles, both to increase intracellular delivery efficiency and to offer protection from degradation.

### 1.3 Scope

Here, we describe the development of a biodegradable cationic polyester for intramuscular delivery of SAM and demonstrate its efficiency in delivering SAM both in vitro to a myoblast mouse cell line and in vivo in mice following intramuscular administration. The resulting nanoparticles formed via a bulk-mixing and dialysis process were demonstrated to be consistent in size as well as stable following lyophilization. This work builds upon research utilizing similar polymers for systemic nucleoside modified mRNA delivery, where prior generation polymers have shown effectiveness for delivery to the lungs and spleen. Kaczmarek et al., 2018; Rui et al., 2022. When used to deliver a rabies antigen encoding SAM, the new nanoparticles enabled protective immunity by eliciting neutralizing serum antibodies at doses of only 200 nanograms of RNA per mouse. Overall, this polymeric nanoparticle platform using rapidly biodegradable cationic esters holds promise as an effective delivery vehicle for intramuscular delivery of large RNA molecules, such as SAM. The hydrophobic PBAEs used here benefit from rapid polymer backbone degradation that limits cytotoxicity, have reduced complexity in the number of components required in the nanoparticle formulation, and as self-assembly is driven by electrostatic interactions with the nucleic acid cargo, can be manufactured flexibly from either bulk batch processes or fluidic mixing. Kaczmarek et al., 2018; Wilson et al., 2017; Hu et al., 2021. Additionally, PBAEs have high avidity to nucleic acids due to the repeating amine groups throughout the polymer, enhanced endosomal escape compared to leading commercially available polymer and lipid-based transfection agents, Rui et al., 2022, and the ability to precisely tune chemical structure to facilitate cell-targeting. Kim et al., 2014. The polymer and RNA self-assemble to small, low polydispersity nanoparticles with high mRNA encapsulation efficiency, and that can be further surface shielded in a modular fashion with a sheddable PEG-lipid in the same manner as commercially approved lipid nanoparticles.

### 1.4 Results and Discussion

1.4.1 Biophysical Properties Characterization ofPBAE Nanoparticles Encapsulating SAM

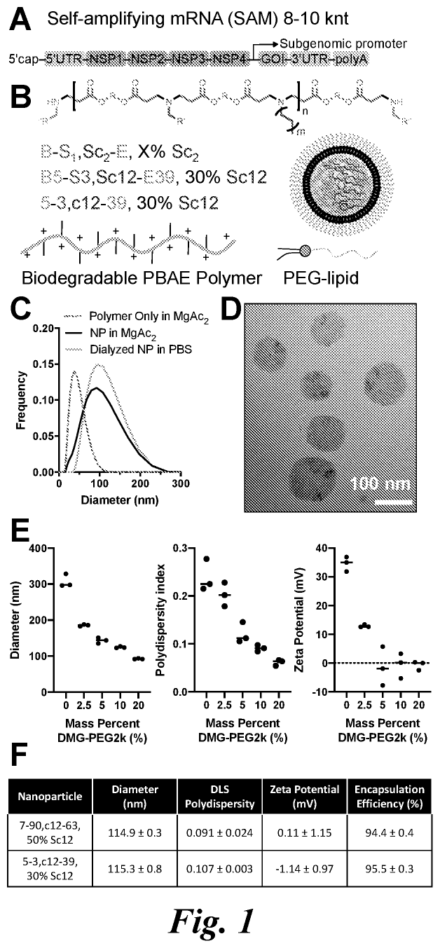

To assess properties of PBAE terpolymers (FIG. 1) with SAM (FIG. 1A), we synthesized a library of PBAEs from small molecule monomers with structure denoted as described (FIG. 1B) following the naming scheme for B: base monomers, S: side-chain monomers, Sc:alkyl-side chain monomers and E: end-cap monomers (FIG. 1B) and using hyphens to separate classes of monomers. Tzeng et al., 2011. While PBAE terpolymers enable co-complexation with nucleic acids using exclusively aqueous buffers for mixing, co-formulation with lipids and a mixing strategy using acidified ethanol and dialysis yielded similar nanoparticle diameters (FIG. 1C) with improved RNA encapsulation as previously observed in related structures. Kaczmarek et al., 2018; Rui et al., 2022; Kaczmarek et al., 2016. Analysis of the PEG-lipid stabilized PBAE terpolymer eGFP SAM nanoparticles by transmission electron microscopy revealed dried nanoparticles of approximately 100 nm in diameter with a spherical shape (FIG. 1D). Inclusion of the alkyl-amine side chain monomer, Eltoukhy et al., 2013, was crucial to enable co-complexation of PEG-lipid molecules for surface shielding of the nanoparticles. We selected use of DMG-PEG2k as a saturated, 14-carbon diacyl lipid for sheddable PEGylation, which has been previously demonstrated to be useful for progressive shedding of the PEG-lipids in vivo to enable cell uptake. Ryals et al., 2020; Zhu et al., 1990. In our experiments using acidified ethanol and dialysis to encapsulate SAM, admixing DMG-PEG2k at a mass percentage between 0-20% resulted in smaller, less polydisperse nanoparticles, with a neutral zeta potential for DMG-PEG2k content ≥5% by mass (FIG. 1E). This approach was applicable to PBAE terpolymers containing 12-carbon alkyl-amine side chain monomers of varied structure including for the two lead nanoparticles later identified (FIG. 1F), 7-90, c12-63, 50% Sc12 and 5-3, c12-39, 30% Sc12. Following screening of a polymer library with diverse structures, both lead nanoparticle formulations had a convergence of biophysical properties when assessed at a 30:1 weight PBAE:weight SAM ratio with the addition of 10% DMG-PEG2k by mass. At these conditions both types of nanoparticles had high encapsulation efficiency (≥94%), a particle size of approximately 115 nm, and neutral zeta potential.

### 1.4.2 PBAE Nanoparticle Library Synthesis and In Vitro Screening

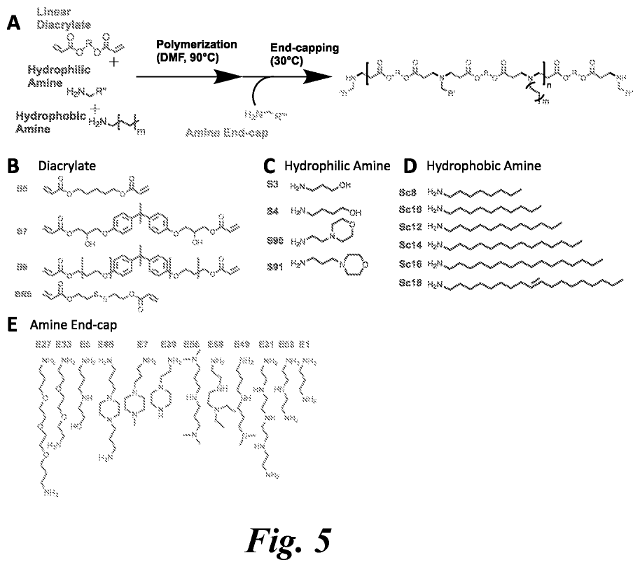

To enable rapid screening of a diverse library of lipophilic 4-component PBAEs (Table 1) for SAM delivery, we adapted a semi-high-throughput strategy we previously employed for screening canonical 3-component PBAE polymers for plasmid DNA delivery. Mishra et al., 2019. This entailed synthesizing acrylate-terminated polymers in mid-size batches in parallel in glass vials and performing the final end-cap monomer reaction in parallel in 384-well plates. Mishra et al., 2019. For this approach, we synthesized sets of PBAEs to be acrylate-terminated (Table 2), characterized the polymers (Table 3), and then performed end-capping reactions with a set of previously identified high efficacy end-cap monomers (FIG. 5). Mishra et al., 2019.

TABLE 1
Monomers used for PBAE Synthesis.

| | | | |
|---|---|---|---|
| | | Cas | Product |
| Chemical Name | Supplier | Number | Number |

Linear Diacrylate Monomers

| | | | | |
|---|---|---|---|---|
| B5 | 1,5-Pentanediol diacrylate | Bajae Labs | 36840- | 9281 |
| | | | 85-4 | |
| B7 | Bisphenol A glycerolate (1 | Sigma | 4687-94- | 411167 |
| | glycerol/phenol) diacrylate | Aldrich | 9 | |
| B9 | Bisphenol A ethoxylate (1.5 | Sigma | 64401- | 413550 |
| | EO/phenol) diacrylate | Aldrich | 02-1 | |
| BR6 | 2,2-disulfanediylbis(ethane-2,1- | Synthesized | N/A | N/A |
| | diyl) diacrylate | according to | | |
| | | Kozielski et | | |
| | | al. 2013 | | |

Hydrophilic Amino Monomers

| | | | | |
|---|---|---|---|---|
| S3 | 3-amino-1-propanol | Alfa Aesar | 156-87-6 | B23041 |
| S4 | 4-amino-1-butanol | Fisher | 13325- | AC176350050 |
| | | Scientific | 10-05 | |
| S90 | 4-(2-Aminoethyl)morpholine | Sigma | 2038-031 | A55004 |
| | | Aldrich | | |
| S91 | 3-Morpholinopropylamine | Sigma | 123-00-2 | A9028 |
| | | Aldrich | | |

Hydrophobic Amino Monomers

| | | | | |
|---|---|---|---|---|
| Sc8 | Octylamine | Sigma | 111-86-6 | O5802 |
| | | Aldrich | | |
| Sc10 | 1-decylamine | Sigma | 2016-57- | 30692 |
| | | Aldrich | 1 | |
| Sc12 | 1-dodecylamine | Alfa Aesar | 124-22-1 | A15515 |
| Sc13 | Tridecylamine | TCI America | 2869-34- | A0762 |
| | | | 3 | |
| Sc14 | Tetradecylamine | Acros | 2016-42- | AC138100500 |
| | | Organics | 4 | |
| Sc16 | Hexadecylamine | Acros | 143-27-1 | AC120510050 |
| | | Organics | | |
| Sc18 | Oleylamine | Sigma | 112-90-3 | O7805 |
| | | Aldrich | | |

End-cap Amino Monomers

| | | | | |
|---|---|---|---|---|
| E1 | 1,3-diaminopropane | Sigma | 109-76-2 | D23602 |
| | | Aldrich | | |
| E6 | 2-(3-Aminopropylamino)ethanol | Sigma | 4461-39- | 9293 |
| | | Aldrich | 6 | |
| E7 | 1-(3-Aminopropyl)-4- | Alfa Aesar | 4572-031 | L04876 |
| | methylpiperazine | | | |
| E27 | 4,7,10-Trioxa-1,13- | Sigma | 4246-51- | 369519 |
| | tridecanediamine | Aldrich | 9 | |
| E31 | Tetraethylenepentamine | Sigma | 1112-57- | T11509 |
| | | Aldrich | 2 | |
| E33 | 2,2′- | Sigma | 929-59-9 | 385506 |
| | (Ethylenedioxy)bis(ethylamine) | Aldrich | | |
| E39 | 1-(2-Aminoethyl)piperazine | Sigma | 140-31-8 | A55209 |
| | | Aldrich | | |
| E49 | N,N- | Sigma | 10563298 | 550019 |
| | Dimethyldipropylenetriamine | Aldrich | | |
| E56 | 3,3′-Iminobis(N,N- | Santa Cruz | 6711484 | SC-231967 |
| | dimethylpropylamine) | | | |
| E58 | N,N-Diethyldiethylenetriamine | Sigma | 24426- | 518832 |
| | | Aldrich | 16-2 | |
| E63 | Diethylentriamine | Sigma | 111-40-0 | 803274 |
| | | Aldrich | | |
| E65 | 1,4-Bis(3- | TCI America | 7209-38- | B1041 |
| | aminopropyl)piperazine | | 3 | |

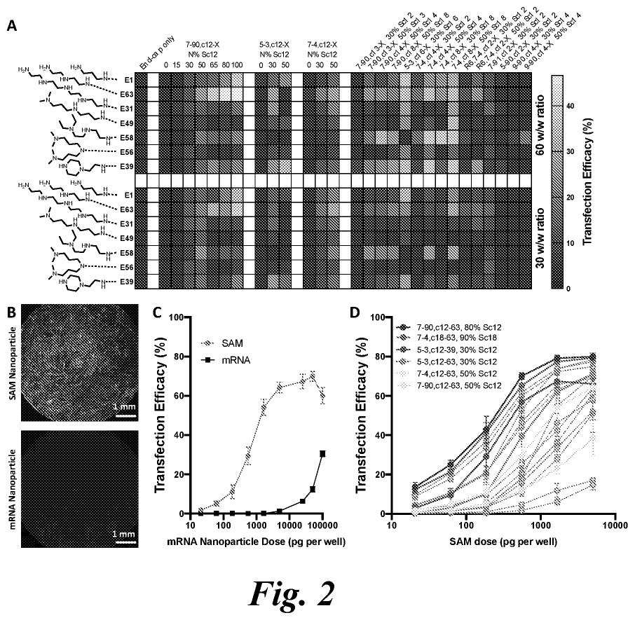

TABLE 2
PBAE synthesis conditions. All were synthesized at 90°
C. for 48 hours in DMF. Arranged to match FIG. 2A.

| Base | Overall | Alkyl | | Synthesis |
|---|---|---|---|---|
| polymer | Vinyl:Amine | Side-chain | Bioreducible | Conc |
| Name | Monomer Ratio | Monomers | Monomers | (mg/mL) |
| 7-90- | 2.173 | 0 | 0 | 400 |
| 7-90,c12- | 2.3 | 15% Sc12 | 0 | 600 |
| 7-90,c12- | 2.3 | 30% Sc12 | 0 | 600 |
| 7-90,c12- | 2.3 | 50% Sc12 | 0 | 600 |
| 7-90,c12- | 2.3 | 65% Sc12 | 0 | 600 |
| 7-90,c12- | 2.3 | 80% Sc12 | 0 | 600 |
| 7-90,c12- | 2.4 | 100% Sc12 | 0 | 300 |
| 5-3- | 2.187 | 0 | 0 | Neat |
| 5-3,c12- | 2.1 | 30% Sc12 | 0 | 600 |
| 5-3,c12- | 2.1 | 50% Sc12 | 0 | 600 |
| 5-3,c12- | 2.1 | 50% Sc12 | 0 | 600 |
| 7-4- | 2.182 | 0 | 0 | 400 |
| 7-4,c12- | 2.205 | 30% Sc12 | 0 | 400 |
| 7-4,c12- | 2.235 | 50% Sc12 | 0 | 400 |
| 7-90,c13- | 2.300 | 30% Sc13 | 0 | |
| 7-90,c13- | 2.3 | 50% Sc13 | 0 | 600 |
| 7-90,c14- | 2.4 | 50% Sc14 | 0 | 600 |
| 7-90,c18- | 2.3 | 50% Sc18 | 0 | 600 |
| 5-3,c16- | 2.1 | 30% Sc16 | 0 | 600 |
| 7-4,c14- | 2.3 | 30% Sc14 | 0 | 600 |
| 7-4,c14- | 2.3 | 50% Sc14 | 0 | 600 |
| 7-4,c18- | 2.3 | 30% Sc18 | 0 | 600 |
| 7-4,c18- | 2.3 | 50% Sc18 | 0 | 600 |
| R6,7-4,c12- | 2.040 | 30% Sc12 | 60% BR6 | 500 |
| R6,7-4,c12- | 2.040 | 50% Sc12 | 60% BR6 | 500 |
| 7-91,c12- | 2.400 | 30% Sc12 | 0 | 400 |
| 5-90,c12- | 2.100 | 30% Sc12 | 0 | 600 |
| 9-90,c14- | 2.4 | 30% Sc14 | 0 | 600 |
| 9-90,c14- | 2.4 | 50% Sc14 | 0 | 600 |
| 7-90,c8- | 2.3 | 30% Sc8 | 0 | 600 |
| 7-90,c10- | 2.3 | 30% Sc10 | 0 | 600 |
| 7-90,c12- | 2.3 | 30% Sc12 | 0 | 600 |
| 7-90,c14- | 2.3 | 30% Sc14 | 0 | 600 |
| 7-90,c16- | 2.3 | 30% Sc16 | 0 | 600 |
| 7-90,c18- | 2.3 | 30% Sc18 | 0 | 600 |

TABLE 3
PBAE characterization properties assessed by gel permeation
chromatography. Arranged to match FIG. 2A.

| Base polymer | GPC MN | GPC MW | GPC MP | |
|---|---|---|---|---|
| Name | (Da) | (Da) | (Da) | GPC PDI |
| 7-90- | 7164 | 20896 | 8158 | 2.917 |
| 7-90,c12- | 7473 | 12142 | 8637 | 1.620 |
| 7-90,c12- | 7299 | 15946 | 7554 | 2.180 |
| 7-90,c12- | 5880 | 17707 | 6394 | 3.012 |
| 7-90,c12- | 5820 | 14530 | 7070 | 2.500 |
| 7-90,c12- | 7217 | 17190 | 10092 | 2.380 |
| 7-90,c12- | 5083 | 9148 | 6083 | 1.800 |
| 5-3- | 5316 | 15919 | 6394 | 2.994 |
| 5-3,c12- | 5446 | 7425 | 6602 | 1.363 |
| 5-3,c12- | 5168 | 6535 | 6404 | 1.264 |
| 5-3,c12- | 7492 | 8253 | 7241 | 1.100 |
| 7-4- | 6851 | 21161 | 7520 | 3.089 |
| 7-4,c12- | 4762 | 10408 | 5844 | 2.186 |
| 7-4,c12- | 4291 | 9892 | 4920 | 2.305 |
| 7-90,c13- | 5746 | 13596 | 6259 | 2.366 |
| 7-90,c13- | 9382 | 45293 | 227896 | 4.830 |
| 7-90,c14- | 7493 | 33512 | 16646 | 4.472 |
| 7-90,c18- | 5816 | 20701 | 6146 | 3.559 |
| 5-3,c16- | 6741 | 7737 | 7006 | 1.150 |
| 7-4,c14- | 5513 | 12770 | 6450 | 2.316 |
| 7-4,c14- | 6589 | 34004 | 187514 | 5.161 |
| 7-4,c18- | 5694 | 14412 | 6611 | 2.531 |
| 7-4,c18- | 7516 | 35262 | 193540 | 4.691 |
| R6,7-4,c12- | 4514 | 11360 | 5270 | 2.517 |
| R6,7-4,c12- | 3348 | 4784 | 3809 | 1.429 |
| 7-91,c12- | 6080 | 17560 | 7299 | 2.876 |
| 5-90,c12- | 6009 | 6485 | 5983 | 1.079 |
| 9-90,c14- | 4290 | 5263 | 5667 | 1.227 |
| 9-90,c14- | 4414 | 5406 | 5818 | 1.225 |
| 7-90,c8- | 4358 | 4993 | 4980 | 1.15 |
| 7-90,c10- | 9330 | 28457 | 16255 | 3.05 |
| 7-90,c12- | 7299 | 15946 | 7554 | 2.18 |
| 7-90,c14- | 7578 | 15557 | 7508 | 2.05 |
| 7-90,c16- | 8774 | 26958 | 14872 | 3.07 |
| 7-90,c18- | 8759 | 26516 | 15197 | 3.03 |

With this strategy we combinatorially synthesized a new library of 196 end-capped PBAEs with varying base monomer and side-chain hydrophobic monomer (horizontal on heatmap), and end-cap monomers (vertical on heatmap) (FIG. 2A). Each polymer was then complexed with SAM at weight:weight ratios of 30 and 60 and assessed for delivery efficiency in differentiated C2C12 murine myoblasts in 384-well plates. Differentiated C2C12 cells were utilized as an in vitro model system to better recapitulate the muscular microenvironment, given the eventual intramuscular delivery route (FIG. 2B-FIG. 2D).

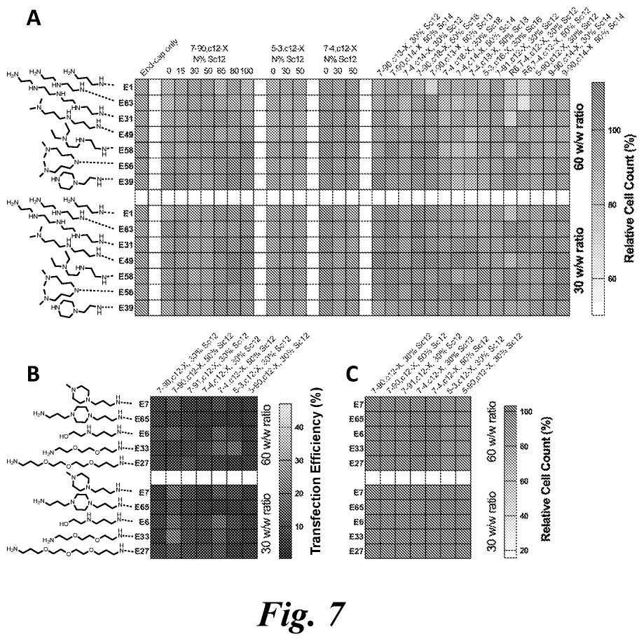

The approach used for initial transfection screening of this PBAE library in C2C12 focused on differential polymer structures (without inclusion of DMG-PEG2k) and aqueous mixing of SAM and PBAE to best enable high-throughput nanoparticle self-assembly in parallel in multi-well plates and assessment of a greater number of polymer structures. In our method validation (FIG. 6), inclusion of DMG-PEG2k or use of the acidified ethanol dialysis encapsulation method did not improve transfection in vitro and similar trends in transfection efficacy were observed regardless of whether the nanoparticles were screened with or without the PEG-lipid. This is likely due to intracellular delivery and endosomal escape to the cytosol both being driven by the PBAE component, which makes up approximately 87-98% of the nanoparticle formulations evaluated by mass. With this approach to screening PBAE structures, we identified multiple PBAE structures effective for SAM delivery to differentiated C2C12 cells in vitro at extremely low doses of only 1 ng/50 μL media in each well of a 384-well plate equivalent to approximately 1 ng per 20,000 cells/well as assessed by nuclei counting. Alternative end-cap monomers previously effective for DNA delivery by PBAEs (including E6 and E7) were also included in an initial trial library of polymers but were not selected for our expanded 384-well screening library (FIG. 7). At these doses, in addition to being efficacious, the PBAE nanoparticles tested were also observed as non-cytotoxic in vitro, with negligible effects on C2C12 viability (FIG. 7).

With the structural variation included in the 384-well screening library, we identified multiple polymer characteristics that improved delivery efficacy. Specifically, increasing alkyl-amine Sc side chain mole fraction (see FIG. 1B 7-90, c12-×, N % Sc12 series and 7-4, c12-X, N % Sc12 series) improved delivery efficacy in vitro even when alkyl-amine side chain mole fraction was increased up to ≥80%. Increasing alkyl-amine side chain length greater than 12 carbons was also beneficial for improving delivery efficacy in vitro. Finally, among the 12 end-cap monomers evaluated, the most effective end-caps across multiple polymers were E63, E58 and E39, which all possess three ionizable amines but differ by their hydrophobicity and ratio of their primary, secondary and tertiary amines following reaction to the acrylate terminated polymers.

To demonstrate increased efficiency of expression with SAM compared to mRNA, PBAE nanoparticles encapsulating eGFP SAM and 5mou-modified eGFP mRNA were prepared in parallel and used to transfect differentiated C2C12 myoblasts at doses across multiple orders of magnitude (FIG. 2B-FIG. 2C). Under these conditions, SAM yielded the same transfection efficacy at a 180-fold lower dose compared to 5-methoxyuridine-modified mRNA in vitro. Recapitulating this two-orders of magnitude level of efficiency improvement in vivo has the potential to enable dose-sparing that could dramatically reduce the supply constraints of mRNA vaccines for global vaccination, as encountered for SARS-CoV-2.

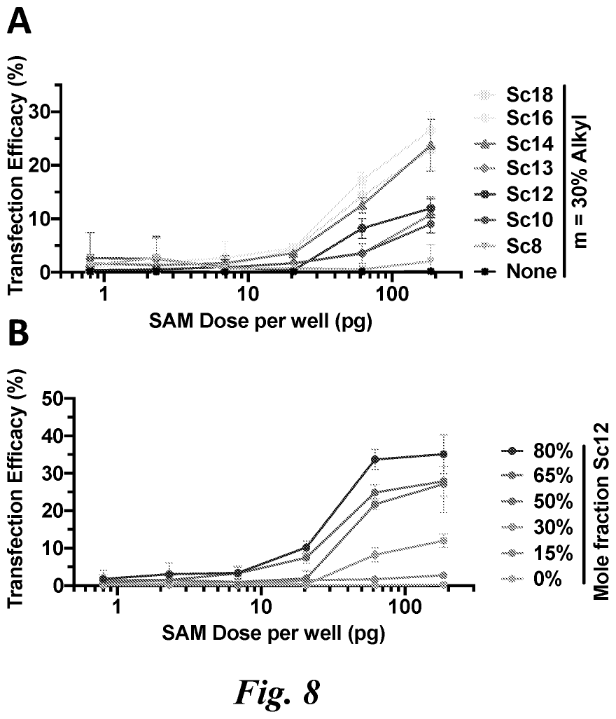

Top nanoparticle formulations identified by screening the 384-well library were evaluated with dose titration for transfection of differentiated C2C12 cells. Among these nanoparticles, lead polymers achieved >70% transfection of cells at a dose of 5 ng/well and >40% transfection at a dose of only 185 μg/well (FIG. 2D). To better understand the influence of the alkyl-amine side-chain monomer on in vitro transfection, we synthesized two series of polymers varying either the length of the alkyl-amine side-chain monomer or the mole fraction (increasing the total polymers in the library evaluated to >200) (FIG. 8). These experiments clearly demonstrated that both increased length of the alkyl-amine side-chain monomer and increased alkyl-amine side-chain mole fraction improved SAM transfection in vitro.

### 1.4.3 Intramuscular (IM) Delivery of PEGylated SAM PBAE Nanoparticles

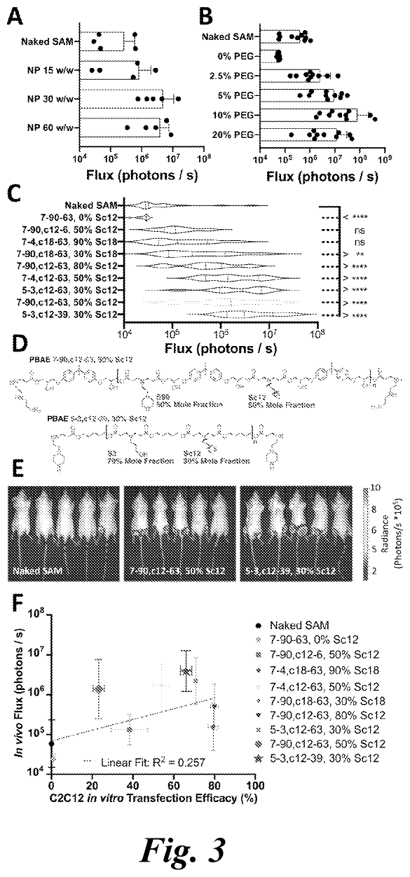

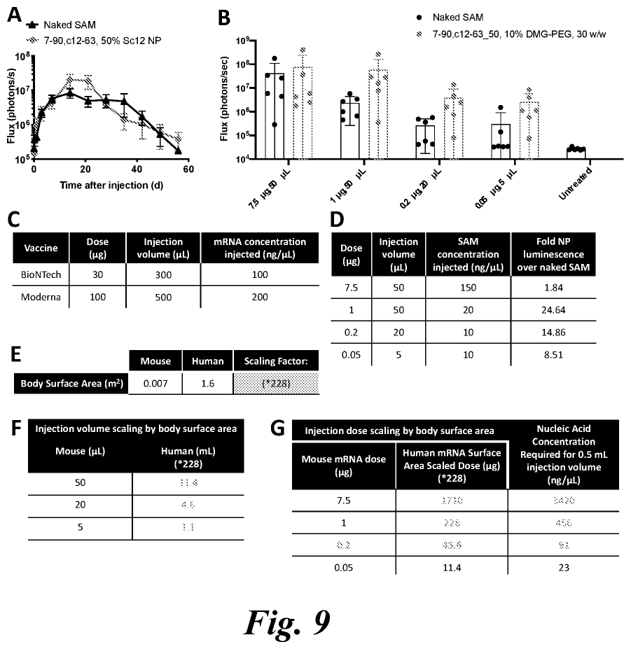

To identify nanoparticles effective for intramuscular SAM delivery in vivo, we used a SAM construct encoding luciferase (FLuc) to rapidly assess overall SAM delivery efficiency when injected intramuscularly with live animal imaging. We first chose a single PBAE nanoparticle formulation and assessed the duration of expression when injected intramuscularly in BALB/c mice (FIG. 9A), observing that peak expression occurred at approximately 10 days post-injection and expression at that dose of SAM persisted through approximately 40 days post-injection. From this experiment, we selected 10 days as the time-point for live animal imaging studies for nanoparticle efficacy assessment in vivo. We further assessed optimal w/w ratio for a single PBAE nanoparticle formulation, evaluating 15, 30 and 60 w/w ratios with DMG-PEG2k included at a 10% mass fraction (FIG. 3A), selecting a 30 w/w ratio as optimal under these conditions. In contrast to in vitro assessment (FIG. 6), where inclusion of DMG-PEG2k moderately reduced transfection, PEGylation of the nanoparticle surface with DMG-PEG2k was shown to be required for efficacy following intramuscular injection of these polymeric nanoparticles (FIG. 3B), presumably by improving their extracellular transport properties. Particles not formulated with DMG-PEG2k yielded lower transfection intramuscularly than naked SAM injected under matched conditions and particles formulated with 10% mass DMG-PEG2k yielding the highest expression. This result was in some ways surprising, as cationic nanoemulsions' Bogers et al., 2015; Archer et al., 2014, cationic liposomes, Blakney et al., 2019, and cationic polyethyleneimine polyplex nanoparticles, Anderson et al., 2014, have previously been demonstrated to be effective for intramuscular administration of nucleic acids despite their positively charged zeta potentials. Yet, the observation that cationic PBAE nanoparticles (even with alkyl-side chains for increased hydrophobicity) yielded no expression with intramuscular administration is consistent with prior observations of PBAE nanoparticles suppressing expression of plasmid DNA when injected intramuscularly, Anderson et al., 2004, unlike when DNA or RNA containing PBAE nanoparticles are injected by other local routes such as intratumorally, Vaughan et al., 2021; Tzeng et al., 2020; Lopez-Bertoni et al., 2018, or intraocularly, Shen et al., 2020, where there is high expression. This is most likely due to the extracellular environment of the muscle, perhaps by unPEGylated PBAE nanoparticles with a positive zeta potential sticking to the high concentration of extracellular matrix proteins in the intramuscular space and failing to reach cell plasma membranes to initiate endocytosis prior to the PBAE backbone beginning hydrolytic degradation. Bhosle et al., 2018. Using polymers with different modes of degradation (like self-immolative oligo (alpha-amino ester)s) may be a possible pathway to improve efficacy without the use of PEG, as it was shown that that such charge-altering releasable transporters (CART) have demonstrated successful intramuscular mRNA delivery at injection doses of 7.5 μg mRNA. McKinlay et al., 2017. In any case, mixing in 10% by mass DMG-PEG2k into the PBAE/SAM nanoparticles during formulation is an easy and modular way to functionalize them to be PEG-shielded for in vivo applications at low doses and the safety of using DMG-PEG2k has been demonstrated in humans via its inclusion in the Moderna COVID-19 vaccine. Baden et al., 2021.

Intramuscular dosing of naked mRNA in mice can present a challenge in reproducibility and utility for assessing delivery efficacy, as murine quadriceps are quite small and naked mRNAs injected in buffer are capable of transfecting muscle cells primarily because of the high hydrostatic pressure achieved when injecting a relatively large volume, without intrinsic ability for the mRNA to safely reach the cytosol itself. Geall et al., 2012; McKinlay et al., 2017; Greig et al., 2014; Gehling et al., 2018; Johannin et al., 1995. Naked nucleic acid expression following intramuscular injection in mice does not reproduce in human patients when typical intramuscular injection volumes in mice (20-50 μL) do not scale with either body surface area (228-fold higher in adult humans, equivalent of 5-11 mL) or body mass and routine intramuscular injection volumes in humans are limited to 500 μL (FIG. 9). The two primary mRNA-based vaccines brought to market for SARS-CoV-2 by BioNTech/Pfizer and Modema use doses of 30 μg and 100 μg in injection volumes of 300 and 500 μL, respectively. Baden et al., 2021; Walsh et al., 2020. Scaling down from these doses and injection volumes to approximate a clinically relevant intramuscular injection in mice is not feasible due to the accuracy and precision constraints of injecting 5 μL intramuscularly. We evaluated using 7-90, c12-63, 50% Sc12 nanoparticles compared to naked SAM injected intramuscularly at a variety of doses between 7.5 to 0.05 μg and injection volumes between 50 μL and 5 μL (FIG. 9). Based on these results we selected a dose of 0.2 μg in 20 μL injection volume (0.01 μg/μL) for all following experiments (FIG. 9).

Using this optimized dose and injection volume of SAM with intramuscular injections, we evaluated 9 different unique PBAE nanostructures prepared at a 30 w/w ratio with SAM and 10% by mass DMG-PEG2k (FIG. 3C). Among these polymers, 6 polymers led to significantly higher luciferase expression than naked SAM, with lead performing formulations of 7-90, c12-63, 50% Sc12 and 5-3, c12-39, 30% Sc12 yielding an increase of 23- and 37-fold higher luminescence over naked SAM (FIG. 3C, FIG. 3D). The structures of these lead polymers were considerably different from one another and demonstrate multiple structural possibilities for high levels of intramuscular delivery efficacy. The 7-90, c12-63 PBAE used a morpholino based ionizable side chain and bisphenol A (BPA) based diacrylate monomer, while the 5-3, c12-39 PBAE used an amino-alcohol based ionizable side chain and a pentanediol based diacrylate. In the context of vaccine development, the higher performing 5-3, c12-39 PBAE may also have a better-tolerability profile due to the avoidance of BPA. Ramos et al., 2003.

One of the primary roles associated with nanoparticle-based encapsulation of SAM and other mRNAs for intramuscular administration is to improve the potency and robustness of delivery over the injection of naked nucleic acids. For the 10 injections shown in representative IVIS images of two lead formulations and naked SAM, PBAE nanoparticle-based encapsulation yielded strongly detectable expression of luciferase 7-9 times out of 10 compared to naked SAM, where only 1/10 injections yielded a strong luminescent signal (FIG. 3E). Even at these low injection doses and volumes, the results show that the biodegradable polymeric nanoparticles can facilitate in vivo intracellular delivery in muscle. It is critical that the PBAE nanoparticles were able to induce significant SAM expression in muscle at modest doses because these small scales have the potential to be translatable to human patients in a manner that larger volume hydrodynamic injection cannot. The hydrodynamic effect by which naked nucleic acids injected intramuscularly in mice mediate effective cytosolic delivery has been demonstrated to have scaling challenges to larger animals like non-human primates. Wells, 2004; Itaka et al., 2010.

Many studies have assessed correlation between transfections in vitro and in vivo for screening different nanoparticle formulations, often revealing low correlation between the optimal materials identified for in vitro and in vivo based delivery. Notably, for lipid nanoparticles with delivery assessed using a barcoded, pooled library approach for delivery to macrophage cell lines in vitro and via intravenous administration in vivo, there was a linear correlation with R²<0.1. Paunovska, 2018. In the context of intramuscular delivery, there have been strategies to improve in vitro models of muscle by using ECM mimicking substrates to influence the mechano-transduction of muscle cells and recapitulate endocytic pathways observed in vivo. Bhosle et al., 2018. While the approach of creating more complex but applicable in vitro culture conditions for identifying nanoparticles effective for in vivo utilization is attractive, implementation can be difficult. For example, the low stiffness (10 kDa) hydrogel system developed by Bhosle et al. 2018 presents challenges for high throughput screening due to its limited applicability to a multiwell plate format. In the current study, PBAE structures were identified by in vitro multiwell plate screening utilizing differentiated C2C12 myoblasts and further evaluated via in vivo intramuscular delivery. Expression following intramuscular administration was weakly positively correlated with in vitro transfection efficacy (R²=0.257, FIG. 3F) and formulations observed to have low transfection efficacy during the screening in vitro also had low transfection in vivo. In vitro screening of the PBAE system showed utility in that all the nanocarriers that achieved greater than 50% transfection of C2C12 myoblasts in vitro also had the capacity to successfully transfect in vivo following incorporation of DMG-PEG2k and IM injection.

1.4.4 Immunization with PBAE Nanoparticle Delivered SAM Improves Rabies Antibody Titers in Mice

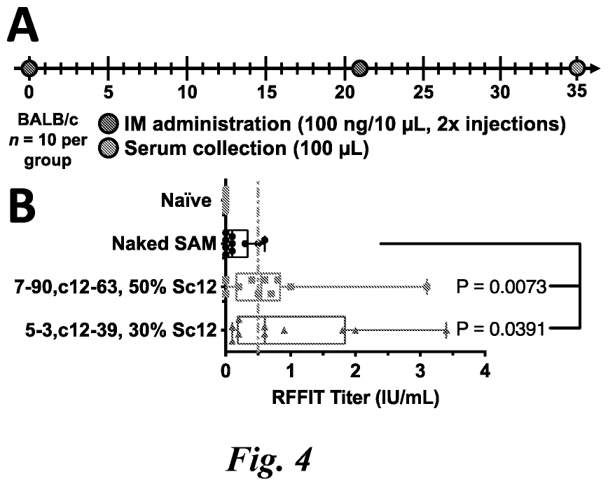

Using a SAM construct encoding rabies virus glycoprotein, we assessed the potential of PBAE nanoparticles to deliver SAM to elicit neutralizing antibodies relative to injections of naked SAM. Lou et al., 2020; Anderluzzi et al., 2020. Using a vaccination schedule of homologous prime/boost separated by three weeks, serum was collected two weeks following boost administration (day 35) (FIG. 4A). Neutralizing antibody titers against live rabies virus were then assessed via rapid fluorescent focus inhibition technique (RFFIT) assay. Bahloul et al., 2005. The RFFIT assay directly assesses the presence of rabies virus neutralizing antibodies (RVnAbs) that can neutralize the rabies virus and prevent infection of healthy cells, providing better correlation for protection than anti-rabies virus glycoprotein binding antibody (bAb) titers measured by ELISA. Bahloul et al., 2005. Comparison between naked SAM and both PBAE NPs tested (7-90, c12-63, 50% Sc12 and 5-3, c12-39, 30% Sc12) RVnAbs levels using the Mann-Whitney test demonstrated statistically significant antibody titer produced after initial immunization and booster injection. An antibody titer above the 0.5 IU/mL threshold (indicated by the dotted line) is considered protective by the assay, Bahloul et al., 2005, demonstrating at the low mRNA doses of 200 ng SAM a seroconversion rate of 6/10 animals for both PBAE NP formulations and 2/10 for naked SAM (FIG. 4B). Thus, both biodegradable polymeric nanoparticle formulations improved the seroconversion of IM injected SAM compared to hydrodynamic injection of naked SAM. These findings merit further investigation as higher doses and additional tuning of intracellular delivery parameters towards antigen-presenting cells could further boost titers. It is also noteworthy that as PBAE nanoparticles are capable of being lyophilized and stored in non-frozen conditions, Guerrero-Cizares et al., 2014, future investigation may prove that they can be beneficial from a supply chain perspective and/or in the development of alternative routes of administration, including via microneedles, which has been demonstrated using PBAEs and plasmid DNA. Qu et al., 2020.

### 1.5 Summary

In this study, we developed biodegradable end-capped lipophilic poly(beta-amino ester) terpolymers to enable the delivery of SAM constructs both in vitro and in vivo for intramuscular vaccination. By screening a library of >200 PBAE terpolymers with varied base polymer structure, differential side chain hydrophobicity and varied end-cap monomers, we identified optimal structural properties to enable highly efficient SAM delivery to myoblasts at sub-nanogram doses per well. In particular, small changes to end-group structure and % of lipophilic side chain could make a profound difference to rates of transfection. Inclusion of alkyl side chains enabled admixing with DMG-PEG2k to yield nanoparticles with high encapsulation efficiency and neutral zeta potential for effective intramuscular administration. Among lead PBAE terpolymers further evaluated by intramuscular injection in mice, optimal PBAE formulations enabled up to 37-fold higher intramuscular expression of SAM compared to injected naked SAM constructs. In vitro screening with C2C12 myoblasts was found to be helpful in identifying polymeric nanocarriers with the capacity for successful intramuscular transfection. Delivery of SAM encoding rabies virus glycoprotein via two different PBAE nanostructures at the low dose of 200 ng mRNA led to protective seroconversion among most animals vaccinated and demonstrated higher humoral immunity compared to injection of naked SAM. This study reveals that biodegradable polymers, as a class of nanomaterials, can be promising delivery vehicles for next-generation mRNA-based vaccines. Although the field is dominated by lipid-based materials for non-viral mRNA delivery, biodegradable polymers have the potential benefits of a broader therapeutic window, ease in manufacturability, possibility for non-frozen supply chain, and efficiency of delivery. This motivates future work in further optimizing dosing, excipients, scale-up, and storage to better realize the potential of this promising class of nanomaterials.

### 1.6 Experimental Section

Materials: Monomers were purchased from vendors listed in Table 1 and used without further purification. Acrylate monomers were stored with desiccant at 4° C., while amine monomers were stored with desiccant at room temperature. mRNA for eGFP (5-methoxyuridine, 5mou) was purchased from TriLink Biotechnologies (L-7201). All solvents were purchased from MilliporeSigma.

### 1.6.1 RNA Synthesis

Self-amplifying mRNA (SAM) is an alphavirus derived mRNA, which comprises genes for nonstructural proteins (NSPs) and a gene of interest (GOI) whose expression was enabled via a subgenomic promoter (FIG. TA). Three SAM constructs were prepared coding for eGFP, firefly luciferase and a dual firefly luciferase-2A-rabies antigen SAM separated by a 2A ribosomal skip site. RNAs were transcribed in vitro from template DNA constructs using T7 polymerase and purified as previously described, Geall et al., 2012, and RNA integrity was validated by agarose gel electrophoresis. Ability of the in vitro transcribed RNAs to self-amplify and express the target antigens was measured in BHK cells as previously described. Maruggi et al., 2022.

### 1.6.2 Polymer Synthesis

Poly(beta-amino ester)s (PBAEs) were synthesized as previously described and shown in FIG. 5 using a two-step Michael addition reaction with a combination of backbone, side chain, and end-cap monomers. Bioreducible monomer-BR6 (2,2-disulfanediylbis(ethane-2,1-diyl) diacrylate) was synthesized according to Kozielski et al., 2013, PBAE polymers were synthesized at the molar ratios of monomers specified in Table 2. The first Michael addition reaction between the backbone and side chain monomers occurred at 90° C. for 48 hr with stirring producing acrylate-terminated base polymers. The second Michael addition reaction occurred in anhydrous tetrahydrofuran (THF) at room temperate for 1 hr with stirring resulting in end-capped PBAEs. These polymers were then precipitated into anhydrous diethyl ether with centrifugations at 3200 rcf and washing twice with anhydrous diethyl ether to purify the polymer. The PBAEs were then dried under vacuum for 48 hrs to eliminate residual diethyl ether, dissolved in anhydrous dimethyl sulfoxide (DMSO) at 100 mg/mL, and stored in aliquots at −20° C. with desiccant to limit freeze/thaw cycles. PBAE nomenclature follows the numbering of diacrylate, hydrophilic amine, hydrophobic amine with percentage, and amine end-cap monomers shown in FIG. 5.

### 1.6.3 Polymer Characterization

Prior to end-capping reactions, acrylate terminated polymers after the first Michael addition reaction were sampled and precipitated twice in anhydrous diethyl ether to yield a neat polymer that was then dissolved in a small amount of anhydrous DMSO-d6. The sampled acrylate-terminated polymers were dried under vacuum for 2 hrs then dissolved in additional DMSO for NMR spectrum analysis of acrylate peaks via Bruker 500 MHz NMR. Similar analysis was done with polymer samples post-end capping to confirm complete reaction by elimination of acrylate peaks between 5.5 and 6.5 ppm. Gel permeation chromatography (GPC) (Waters, Milford, MA) was also used to characterize the MN, Mw and polydispersity index (PDI) relative to linear polystyrene standards for the sampled acrylate-terminated polymers as previously described. Wilson et al., 2019.

### 1.64 Encapsulation Efficiency Assay

Loading efficiency of SAM loaded into the nanoparticles was analyzed using the commercial Invitrogen Ribogreen RNA analysis kit (ThermoFisher) as described previously. Kaczmarek et al., 2016. Nanoparticles were complexed using two different PBAE formulations (7-90, c12-63, 50% Sc12 and 5-3, c12-39, 30% Sc12) to encapsulate SAM with 10 m % DMG-PEG2k and underwent dialysis. Nanoparticles were then diluted to approximately 1 ng/μL SAM in PBS pH 7.4 buffer. Standards using the SAM molecules were between 0.125 and 2 ng/μL. Prepared nanoparticles were then mixed with either PBS buffer or 10 mg/mL heparin in TE buffer with the later disrupting the polymer binding allowing for the release of SAM. The nanoparticles were incubated in the buffer for 15 mins at 37° C. and then diluted Ribogreen reagent was added and incubated for 3 mins at 37° C. Fluorescence of Ribogreen was measured using a plate reader (Biotek Synergy) at 500 nm/525 nm excitation/emission according to the supplied protocol to determine encapsulation efficiency.

### 1.65 Nanoparticle Preparation

Nanoparticles were prepared without incorporation of lipid-PEG for high-throughput screening of the polymers' ability to facilitate intracellular delivery in vitro or by adding DMG-PEG2k as an extra component to neutralize surface charge followed by dialysis as previously described. Kaczmarek et al., 2018. For transfections in 96-well plates, nanoparticles were formed by dissolving synthesized PBAE polymers in DMSO and eGFP SAM separately in 25 mM NaAc pH 5.0 buffer and combining them at a 1:1 volume ratio. The mixture was incubated at room temperature for 10 mins to allow for self-assembly into nanoparticles. For transfections in 384-well plates, nanoparticles were formed by resuspending synthesized PBAE polymers in 25 mM NaAc pH 5.0 buffer in parallel using a ViaFlo 384 (Integra Biosciences). Resuspended PBAE polymer was then mixed in parallel with SAM to yield a final nucleic acid concentration of 0.03 μg/μL in a 384 polypropylene nanoparticle source plate.

### 1.6.6 Nanoparticle Characterization

For nanoparticle characterization via dynamic light scattering, SAM and PBAE polymer were prepared using DMG-PEG2k and dialysis. Kaczmarek et al., 2018. The Z-average hydrodynamic diameters and zeta potential of the nanoparticle formulations in 25 mM NaAc and in six-fold dilution using isotonic 150 mM PBS pH 7.4 buffer at 25° C., respectively, were measured by dynamic light scattering (DLS) using a Zetasizer Pro (Malvern Instruments, Malvern, UK) with a 173° detection angle. For transmission electron microscopy (TEM), nanoparticles were prepared at 30 w/w ratio with 10 m % DMG-PEG2k using dialysis against PBS for 75 minutes. Twenty microliters of nanoparticles were used to coat a corona plasma-treated carbon film 400 square mesh TEM grid for 60 mins. Grids were then briefly washed in ultrapure water to eliminate excess dried salt crystals and dried under vacuum before acquiring images using a Philips CM120 (Philips Research, Briarcliffs Manor, New York).

### 1.6.7 Cell Culture

C2C12 murine myoblast cells were purchased from ATCC (Manassas, VA, CRL-1772) and expanded in DMEM supplemented with 10% heat-inactivated fetal bovine serum and 1% penicillin/streptomycin. For differentiation to myotube-like cells, C2C12 cells were plated at a density of 31,250 cells/cm² in tissue culture plates in DMEM supplemented with 2% horse serum, 1% insulin, selenium and transferrin (ITS, 41400045, ThermoFisher). For 96-well plate transfection efficacy experiments, cells were plated on CytoOne 96-well tissue culture plates (USA Scientific, Ocala, FL) 4 days prior to transfection with 12,000 cells/well in 100 μL complete differentiation media and media was changed on days 2 and 4. For noted 384-well plate transfection experiments, C2C12 cells were plated at 2,500 cells/well in 50 μL complete differentiation media in 384-well tissue culture plates (Santa Cruz, sc-206081) 2 days prior to transfection and media was replaced on the day of transfection. Cells were confirmed periodically to be mycoplasma negative via the MycoAlert test (Lonza).

### 1.6.8 In Vitro Transfection

For 96-well transfections, 20 μL of SAM nanoparticle dilution with nucleic acid concentration between 1.03-250 μg/μL was added to each well of cells in 100 μL of complete media for a 2 hr incubation before changing media. Cell viability 24 hrs post-transfection was assessed using the MTS Celltiter 96 Aqueous One (Promega, Madison, WI) cell proliferation assay. For 384-well transfections, 5 μL of nanoparticle dilution was added to each well containing cells in 50 μL of complete media and left to incubate for 2 hr before a media change. Percent transfection efficiency was assessed after 48 hrs by staining nuclei with Hoechst stain and imaged for eGFP expression using a Cellomics Arrayscan VTI (Thermo Fisher Scientific, Laguna Hills, CA), an automated fluorescence-based high-content screening imaging system.

### 1.6.9 In Vivo Experiments

Animal work was performed in compliance with an approved protocol by the Johns Hopkins University Animal Care and Use Committee (ACUC). Female BALB/c mice, 6-8 weeks old were purchased from The Jackson Laboratory and maintained in accordance with the JHH animal care facility. For in vivo transfection analysis, nanoparticles were made with luciferase (FLuc) SAM and PBAE polymers and injected intramuscularly in mice at 0.2 μg dose for bioluminescent luciferase expression assessment at specified time points. For assessment ofluciferase expression, mice were injected intraperitoneally (i.p.) with 100 μL of 150 mg/kg d-luciferin (potassium salt solution in 1×PBS; Cayman Chemical Company, Ann Arbor, MI). After 7 min, mice were anesthetized using isoflurane and imaged using an In Vivo Imaging System (IVIS Spectrum; PerkinElmer, Shelton, CT) to measure bioluminescence. For the rabies vaccination studies, 6-8 weeks old female BALB/c mice were each injected intramuscularly with nanoparticles carrying SAM encoding both rabies virus glycoprotein antigen and the luciferase reporter protein (0.1 μg in 10 μL in opposite quadriceps for 0.2 μg total dose) on day 0 followed by a booster on day 21. Serum was then collected from the mice at day 35, 14 days after the booster vaccination.

### 1.6.10 Neutralizing Antibody Titer Assay

Serum samples were analyzed for rabies virus neutralizing antibody (RVNA) titer using a rapid fluorescent foci inhibition test (RFFIT) at the Kansas State University Rabies Laboratory. Bahloul et al., 2005. Serum was first diluted five-fold and then serially five-fold before incubating with live rabies virus. Cultured cells were then combined with the serum dilutions with virus to test for protection resulting from RVNA presence via a titer value calculated from the percent of infected cells. KSU Rabies Laboratory and the World Health Organization (WHO) report a titer of 0.5 international units per millimeter (IU/mL) as a protective response resulting from rabies vaccination and this level is indicated on data plots with a dotted line.

### 1.6.11 Data Analysis and Statistics

Cellomics HCS Studio (Thermo Fisher) was used for image acquisition-based in vitro transfection analysis as previously described. Mishra et al., 2019. Polymer structures were characterized in ChemDraw (Perkin Elmer, Boston, MA) and Marvin (ChemAxon, Cambridge, MA) to determine log P and log D values. Prism 8 (GraphPad, La Jolla, CA) was used for all statistical analyses and curve plotting. Unless otherwise specified, statistical tests were performed with a global alpha value of 0.05. Unless otherwise stated, absence of statistical significance markings where a test was stated to have been performed signified no statistical significance. Statistical significance was denoted as follows: *p<0.05; **p<0.01; ***p<0.001; ****p<0.0001.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
D. V Parums, Medical Science Monitor: International Medical Journal of Experimental and Clinical Research 2021, 27, e932915.
A. K. Blakney, S. Ip, A. J. Geall, Vaccines (Basel) 2021, 9, 97.
F. P. Polack, S. J. Thomas, N. Kitchin, J. Absalon, A. Gurtman, S. Lockhart, J. L. Perez, G. Përez Marc, E. D. Moreira, C. Zerbini, R. Bailey, K. A. Swanson, S. Roychoudhury, K. Koury, P. Li, W. v Kalina, D. Cooper, R. W. Frenck, L. L. Hammitt, Ö. Tureci, H. Nell, A. Schaefer, S. Ünal, D. B. Tresnan, S. Mather, P. R. Dormitzer, U. Şahin, K. U. Jansen, W. C. Gruber, C4591001 Clinical Trial Group, N Engl J Med 2020, 383, 2603.
L. R. Baden, H. M. el Sahly, B. Essink, K. Kotloff, S. Frey, R. Novak, D. Diemert, S. A. Spector, N. Rouphael, C. B. Creech, J. McGettigan, S. Khetan, N. Segall, J. Solis, A. Brosz, C. Fierro, H. Schwartz, K. Neuzil, L. Corey, P. Gilbert, H. Janes, D. Follmann, M. Marovich, J. Mascola, L. Polakowski, J. Ledgerwood, B. S. Graham, H. Bennett, R. Pajon, C. Knightly, B. Leav, W. Deng, H. Zhou, S. Han, M. Ivarsson, J. Miller, T. Zaks, New England Journal of Medicine 2021, 384, 403.
L. A. Brito, S. Kommareddy, D. Maione, Y. Uematsu, C. Giovani, F. Berlanda Scorza, G. R. Otten, D. Yu, C. W. Mandl, P. W. Mason, P. R. Dormitzer, J. B. Ulmer, A. J. Geall, Self-Amplifying mRNA Vaccines, Vol. 89, Elsevier Ltd, 2015.
A. J. Geall, A. Verma, G. R. Otten, C. A. Shaw, A. Hekele, K. Banerjee, Y. Cu, C. W. Beard, L. A. Brito, T. Krucker, D. T. O'Hagan, M. Singh, P. W. Mason, N. M. Valiante, P. R. Dormitzer, S. W. Barnett, R. Rappuoli, J. B. Ulmer, C. W. Mandl, Proceedings of the National Academy of Sciences 2012, 109, 14604.
A. B. Vogel, L. Lambert, E. Kinnear, D. Busse, S. Erbar, K. C. Reuter, L. Wicke, M. Perkovic, T. Beissert, H. Haas, S. T. Reece, U. Sahin, J. S. Tregoning, Molecular Therapy 2018, 26, 446.
N. Pardi, M. J. Hogan, D. Weissman, Curr Opin Immunol 2020, 65, 14.
A. K. Blakney, P. F. McKay, B. I. Yus, Y. Aldon, R. J. Shattock, Gene Therapy 2019, 26, 363.
G. Lou, G. Anderluzzi, S. T. Schmidt, S. Woods, S. Gallorini, M. Brazzoli, F. Giusti, I. Ferlenghi, R. N. Johnson, C. W. Roberts, D. T. O'Hagan, B. C. Baudner, Y. Perrie, Journal of Controlled Release 2020, 325, 370.
J. S. Chahal, O. F. Khan, C. L. Cooper, J. S. McPartlan, J. K. Tsosie, L. D. Tilley, S. M. Sidik, S. Lourido, R. Langer, S. Bavari, H. L. Ploegh, D. G. Anderson, Proceedings of the National Academy of Sciences 2016, 113, E4133.
   W. M. Bogers, H. Oostermeijer, P. Mooij, G. Koopman, E. J. Verschoor, D. Davis, J. B. Ulmer, L. A. Brito, Y. Cu, K. Banerjee, G. R. Otten, B. Burke, A. Dey, J. L. Heeney, X. Shen, G. D. Tomaras, C. Labranche, D. C. Montefiori, H. X. Liao, B. Haynes, A. J. Geall, S. W. Barnett, Journal of Infectious Diseases 2015, 211, 947.
G. Anderluzzi, G. Lou, S. Gallorini, M. Brazzoli, R. Johnson, D. T. O'Hagan, B. C. Baudner, Y. Perrie, Vaccines (Basel) 2020, 8, 212.
A. J. Geall, A. Verma, G. R. Otten, C. A. Shaw, A. Hekele, K. Banerjee, Y. Cu, C. W. Beard, L. A. Brito, T. Krucker, D. T. O'Hagan, M. Singh, P. W. Mason, N. M. Valiante, P. R. Dormitzer, S. W. Barnett, R. Rappuoli, J. B. Ulmer, C. W. Mandl, Proceedings of the National Academy of Sciences 2012, 109, 14604.
   J. Archer, S. W. Barnett, A. Lilja, M. Singh, T. Carsillo, P. W. Mason, G. R. Otten, N. M. Valiante, L. A. Brito, M. Schaefer, C. W. Mandl, A. Seubert, M. Chan, P. R. Dormitzer, A. J. Geall, C. W. Beard, A. K. Dey, C. A. Shaw, A. Hekele, J. B. Ulmer, D. T. O'Hagan, Molecular Therapy 2014, 22, 2118.

### J. C. Kaczmarek, K. J. Kauffman, O. S. Fenton, K. Sadtler, A. K. Patel, M. W. Heartlein, F. DeRosa, D. G. Anderson, Nano Letters 2018, 18, 6449.

J. Karlsson, K. R. Rhodes, J. J. Green, S. Y. Tzeng, Expert Opin Drug Deliv 2020, 17, 1395.
D. G. Anderson, D. M. Lynn, R. Langer, Angew Chem Int Ed Engl 2003, 42, 3153.

### D. R. Wilson, M. P. Suprenant, J. H. Michel, E. B. Wang, S. Y. Tzeng, J. J. Green, Biotechnology and Bioengineering 2019, 116, 1220.

J. C. Sunshine, M. I. Akanda, D. Li, K. L. Kozielski, J. J. Green, Biomacromolecules 2011, 12, 3592.
J. Kim, J. C. Sunshine, J. J. Green, Bioconjug Chem 2014, 25, 43.
   Y. Rui, D. R. Wilson, S. Y. Tzeng, H. M. Yamagata, D. Sudhakar, M. Conge, C. A. Berlinicke, D. J. Zack, A. Tuesca, J. J. Green, Sci Adv 2022, 8, eabk2855.
R. B. Shmueli, J. C. Sunshine, Z. Xu, E. J. Duh, J. J. Green, Nanomedicine: Nanotechnology, Biology, and Medicine 2012, 8, 1200.
J. H. Erasmus, J. Archer, J. Fuerte-Stone, A. P. Khandhar, E. Voigt, B. Granger, R. G. Bombardi, J. Govero, Q. Tan, L. A. Durnell, R. N. Coler, M. S. Diamond, J. E. Crowe, S. G. Reed, L. B. Thackray, R. H. Carnahan, N. Van Hoeven, Molecular Therapy Methods and Clinical Development 2020, 18, 402.
D. R. Wilson, A. Mosenia, M. P. Suprenant, R. Upadhya, D. Routkevitch, R. A. Meyer, A. Quinones-Hinojosa, J. J. Green, Journal of Biomedical Materials Research Part A 2017, 105, 1813.
Y. Hu, Y. Zhu, N. D. Sutherland, D. R. Wilson, M. Pang, E. Liu, J. R. Staub, C. A. Berlinicke, D. J. Zack, J. J. Green, S. K. Reddy, H. Q. Mao, Nano Letters 2021, 21, 5697.
S. Y. Tzeng, H. Guerrero-Cizares, E. E. Martinez, J. C. Sunshine, A. Quiñones-Hinojosa, J. J. Green, Biomaterials 2011, 32, 5402.
J. C. Kaczmarek, A. K. Patel, K. J. Kauffman, O. S. Fenton, M. J. Webber, M. W. Heartlein, F. DeRosa, D. G. Anderson, Angew Chem Int Ed Engl 2016, 55, 13808.
A. A. Eltoukhy, D. Chen, C. A. Alabi, R. Langer, D. G. Anderson, Advanced Materials 2013, 25, 1487.
R. C. Ryals, S. Patel, C. Acosta, M. McKinney, M. E. Pennesi, G. Sahay, Plos one 2020, 15, e0241006.

### X. Zhu, W. Tao, D. Liu, J. Wu, Z. Guo, X. Ji, Z. Bharwani, L. Zhao, X. Zhao, O. C. Farokhzad, J. Shi, Theranostics 2017, 7, 1990.

### B. Mishra, D. R. Wilson, S. R. Sripathi, M. P. Suprenant, Y. Rui, K. J. Wahlin, C. A. Berlinicke, J. J. Green, D. J. Zack, Regen Eng Transl Med 2019, 6, 273.

D. G. Anderson, W. Peng, A. Akinc, N. Hossain, A. Kohn, R. Padera, R. Langer, J. A. Sawicki, Proceedings of the National Academy of Sciences 2004, 101, 16028.

### H. J. Vaughan, C. G. Zamboni, N. P. Radant, P. Bhardwaj, E. R. Lechtich, L. F. Hassan, K. Shah, J. J. Green, Molecular Therapy—Oncolytics 2021, 21, 377.

S. Y. Tzeng, K. K. Patel, D. R. Wilson, R. A. Meyer, K. R. Rhodes, J. J. Green, Proc Natl Acad Sci USA 2020, 117, 4043.

### H. Lopez-Bertoni, K. L. Kozielski, Y. Rui, B. Lal, H. Vaughan, D. R. Wilson, N. Mihelson, C. G. Eberhart, J. Laterra, J. J. Green, Nano Lett 2018, 18, 4086.

J. Shen, J. Kim, S. Y. Tzeng, K. Ding, Z. Hafiz, D. Long, J. Wang, J. J. Green, P. A. Campochiaro, Science Advances 2020, 6, eaba1606.

### S. M. Bhosle, K. H. Loomis, J. L. Kirschman, E. L. Blanchard, D. A. Vanover, C. Zurla, D. Habrant, D. Edwards, P. Baumhof, B. Pitard, P. J. Santangelo, Biomaterials 2018, 159, 189.

C. J. McKinlay, J. R. Vargas, T. R. Blake, J. W. Hardy, M. Kanada, C. H. Contag, P. A. Wender, R. M. Waymouth, Proc Natl Acad Sci USA 2017, 114, E448.
J. A. Greig, H. Peng, J. Ohlstein, C. A. Medina-Jaszek, O. Ahonkhai, A. Mentzinger, R. L. Grant, S. Roy, S. J. Chen, P. Bell, A. P. Tretiakova, J. M. Wilson, PLOS ONE 2014, 9, e112268.
A. M. Gehling, K. Kuszpit, E. J. Bailey, K. H. Allen-Worthington, D. P. Fetterer, P. J. Rico, T. M. Bocan, C. C. Hofer, J Am Assoc Lab Anim Sci 2018, 57, 35.
F. W. Johanning, R. M. Conry, A. F. LoBuglio, M. Wright, L. A. Sumerel, M. J. Pike, D. T. Curiel, Nucleic Acids Research 1995, 23, 1495.
E. E. Walsh, R. W. Frenck, A. R. Falsey, N. Kitchin, J. Absalon, A. Gurtman, S. Lockhart, K. Neuzil, M. J. Mulligan, R. Bailey, K. A. Swanson, P. Li, K. Koury, W. Kalina, D. Cooper, C. Fontes-Garfias, P.-Y. Shi, O. Tureci, K. R. Tompkins, K. E. Lyke, V. Raabe, P. R. Dormitzer, K. U. Jansen, U. Sahin, W. C. Gruber, New England Journal of Medicine 2020, 383, 2439.
J. G. Ramos, J. Varayoud, L. Kass, H. Rodriguez, L. Costabel, M. Muñoz-de-Toro, E. H. Luque, Endocrinology 2003, 144, 3206.
D. J. Wells, Molecular Therapy 2004, 10, 207.
K. Itaka, K. Osada, K. Morii, P. Kim, S.-H. Yun, K. Kataoka, Journal of controlled release 2010, 143, 112.
   K. Paunovska, C. D. Sago, C. M. Monaco, W. H. Hudson, M. G. Castro, T. G. Rudoltz, S. Kalathoor, D. A. Vanover, P. J. Santangelo, R. Ahmed, A. v. Bryksin, J. E. Dahlman, Nano Letters 2018, 18, 2148.
C. Bahloul, D. Taieb, B. Kaabi, M. F. Diouani, S. Ben Hadjahmed, Y. Chtourou, B. I. B'chir, K. Dellagi, Epidemiology & Infection 2005, 133, 749.
H. Guerrero-Cizares, S. Y. Tzeng, N. P. Young, A. O. Abutaleb, A. Quiñones-Hinojosa, J. J. Green, ACS Nano 2014, 8, 5141.
M. Qu, H.-J. Kim, X. Zhou, C. Wang, X. Jiang, J. Zhu, Y. Xue, P. Tebon, S. A. Sarabi, S. Ahadian, Nanoscale 2020, 12, 16724.
A. J. Geall, A. Verma, G. R. Otten, C. A. Shaw, A. Hekele, K. Banerjee, Y. Cu, C. W. Beard, L. A. Brito, T. Krucker, D. T. O'Hagan, M. Singh, P. W. Mason, N. M. Valiante, P. R. Dormitzer, S. W. Barnett, R. Rappuoli, J. B. Ulmer, C. W. Mandl, Proc Natl Acad Sci USA 2012, 109, 14604.
G. Maruggi, C. P. Mallett, J. W. Westerbeck, T. Chen, G. Lofano, K. Friedrich, L. Qu, J. T. Sun, J. McAuliffe, A. Kanitkar, K. T. Arrildt, K.-F. Wang, I. McBee, D. McCoy, R. Terry, A. Rowles, M. A. Abrahim, M. A. Ringenberg, M. J. Gains, C. Spickler, X. Xie, J. Zou, P.-Y. Shi, T. Dutt, M. Henao-Tamayo, I. Ragan, R. A. Bowen, R. Johnson, S. Nuti, K. Luisi, J. B. Ulmer, A.-M. Steff, R. Jalah, S. Bertholet, A. H. Stokes, D. Yu, Mol Ther 2022, 30, 1897.
K. L. Kozielski, S. Y. Tzeng, J. J. Green, Chem Commun (Camb) 2013, 49, 5319.
D. R. Wilson, Y. Rui, K. Siddiq, D. Routkevitch, J. J. Green, Molecular Pharmaceutics 2019, 16, 655.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A nanoparticle comprising a compound of formula (I) and one or more nucleic acids: 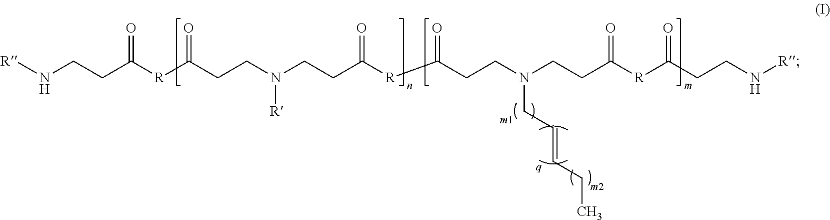
wherein:
m and n are each independently an integer from 1 to 10,000;
m1 is an integer selected from 0, 1, 2, 3, 4, 5, 6, 7, 8, 9, and 10;
m2 is an integer selected from the group consisting of 1, 2, 3, 4, 5, 6, 7, 8, 9, 10, 11, 12, 13, 14, 15, 16, 17, 18, 19, and 20;
q is an integer selected from 0 or 1;
wherein —(CH₂)ₘ₁—(C═C)_{q}—(CH₂)ₘ₂—CH₃ comprises a hydrophobic sidechain;
R comprises a divalent radical comprising a biodegradable ester linkage and/or a bioreducible disulfide linkage;
R′ is hydrophilic sidechain comprising a monovalent radical derived from a hydrophilic amine monomer;
R″ is monovalent radical derived from an amine-containing end capping group; and pharmaceutically acceptable salts thereof.

2. The nanoparticle of claim 1, wherein:
(a) R is selected from the group consisting of: 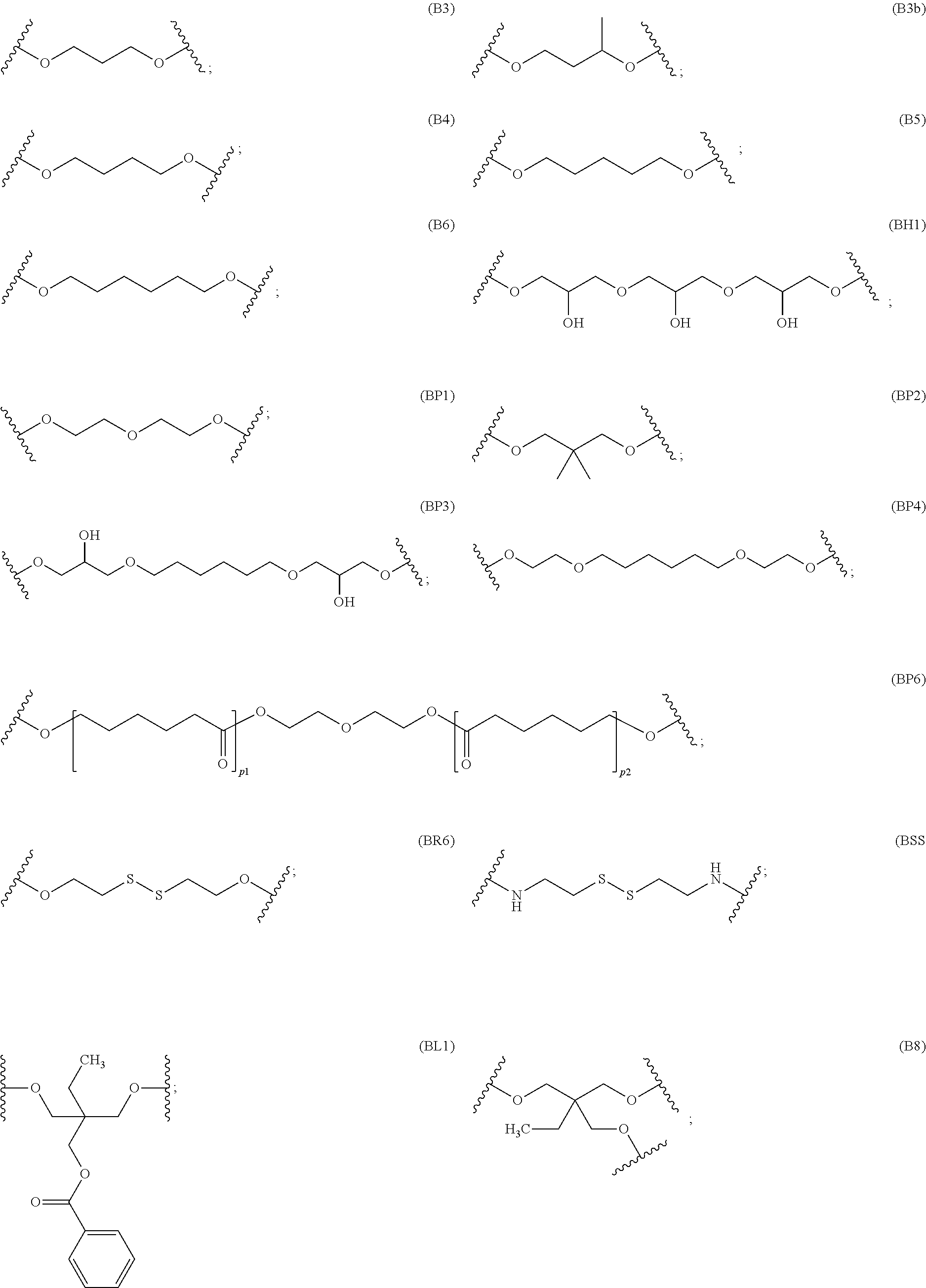 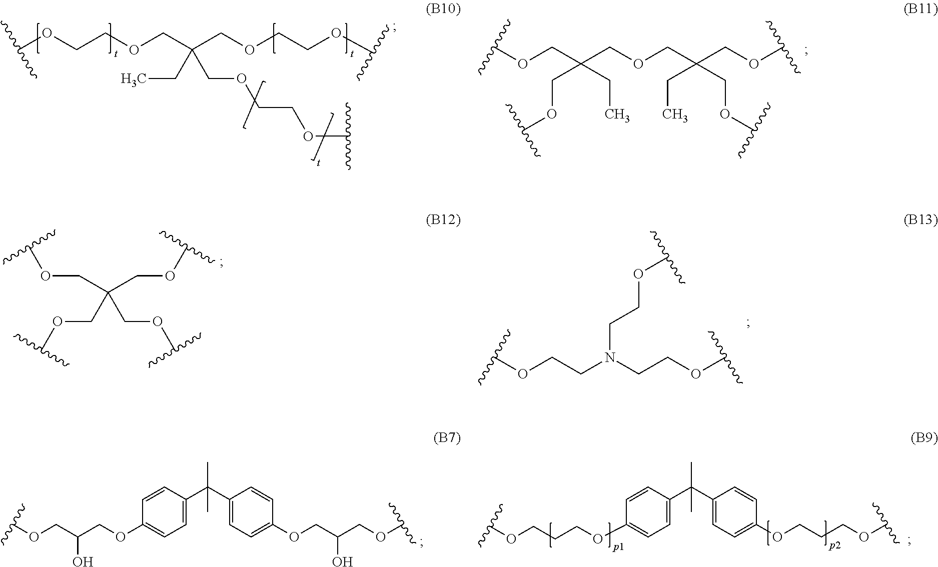
wherein each p1, p2, and t is independently an integer selected from the group consisting of 1, 2, 3, 4, 5, 6, 7, 8, 9, and 10;
(b) R′ is selected from the group consisting of: 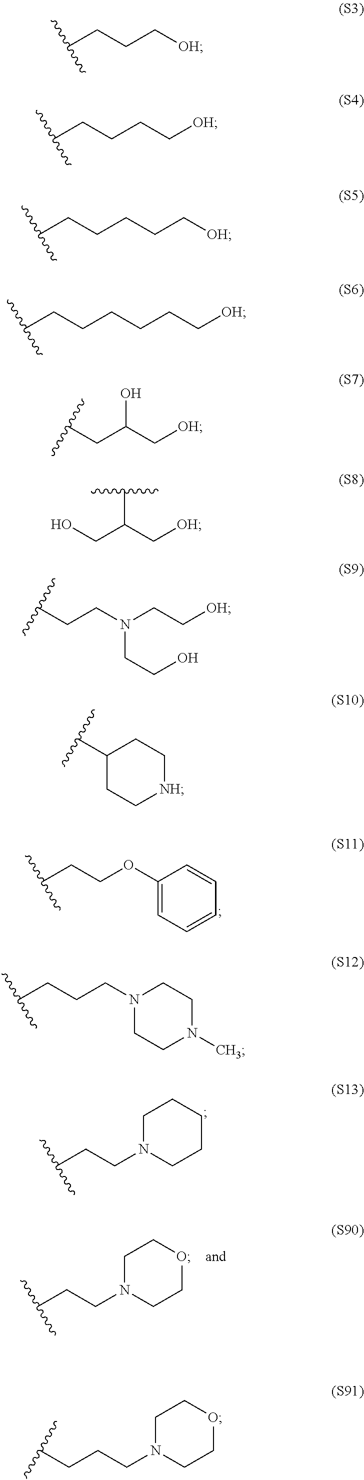
(c) R″ is selected from the group consisting of: 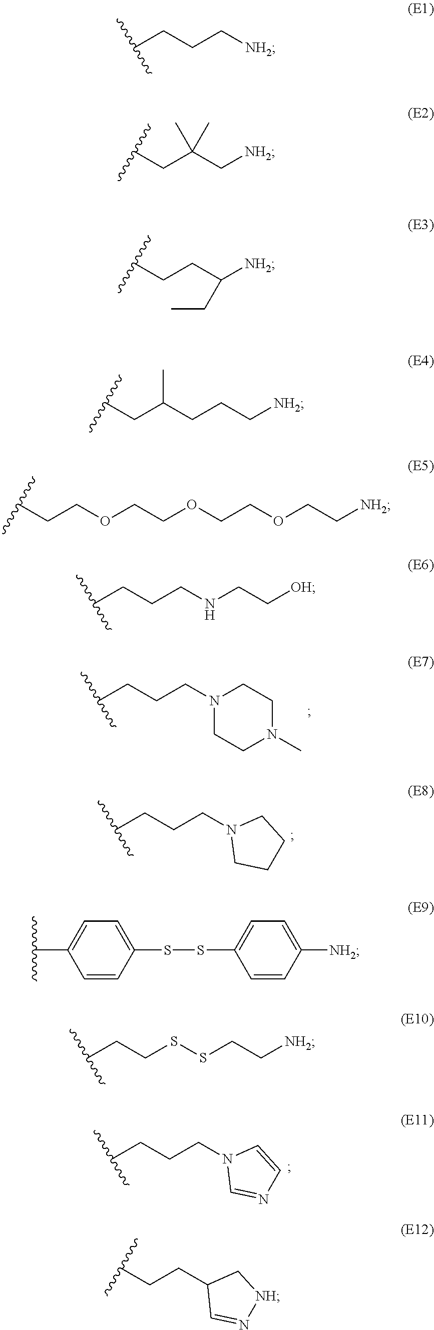 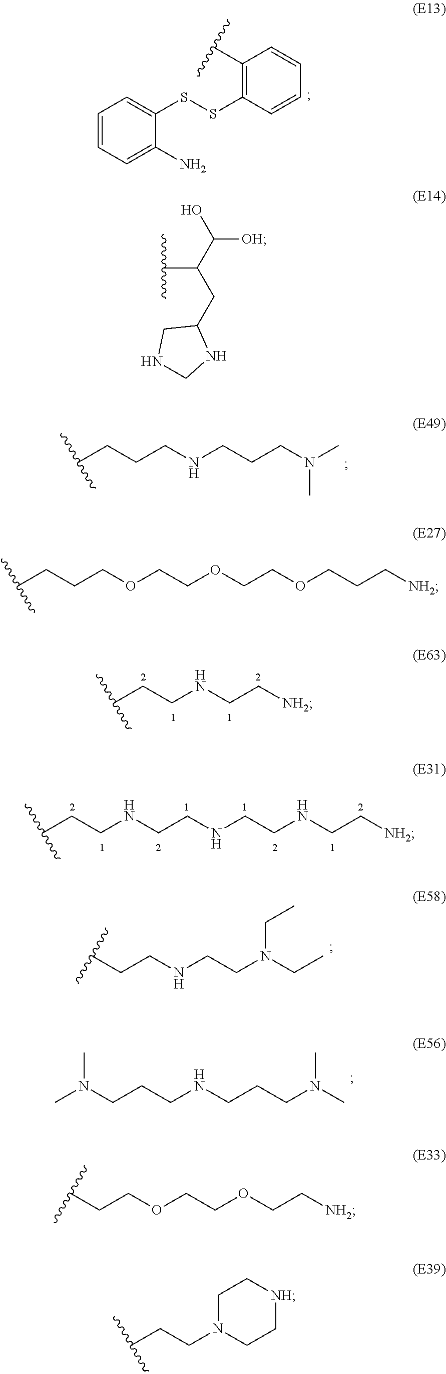 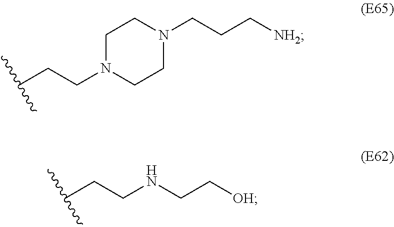 [SMILES: *CCN1CCN(CCCN)CC1 ; *CCNCCO] 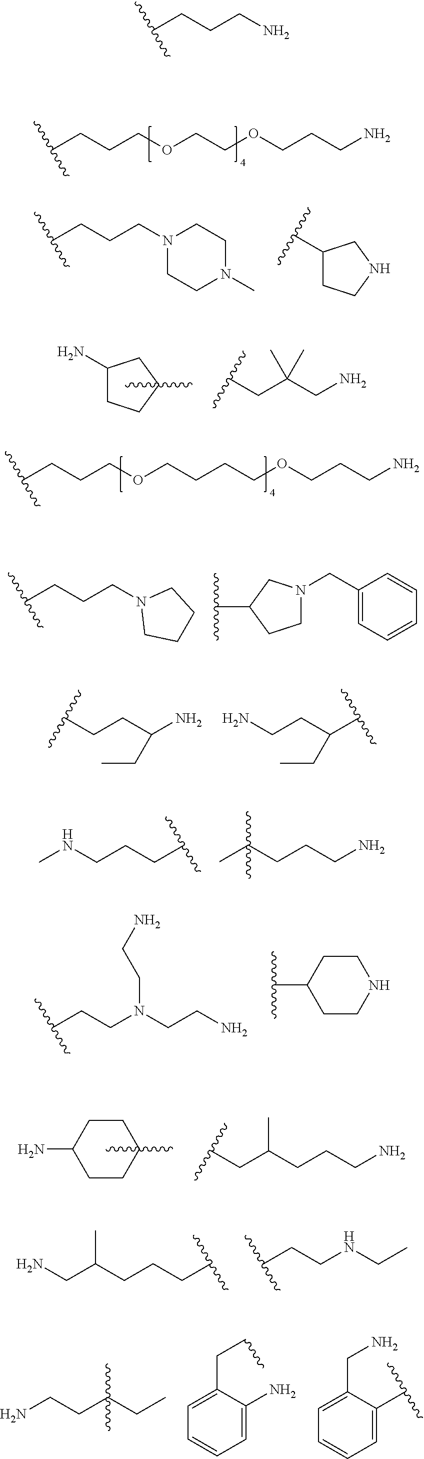 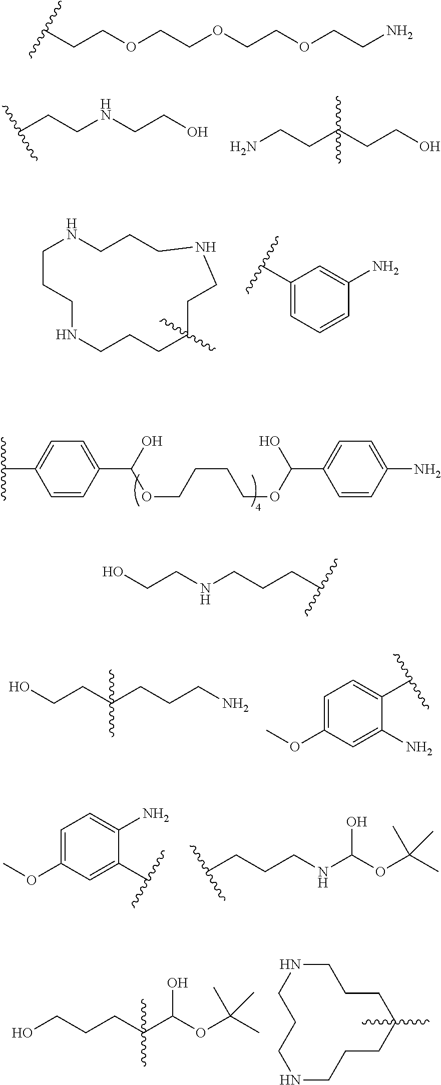 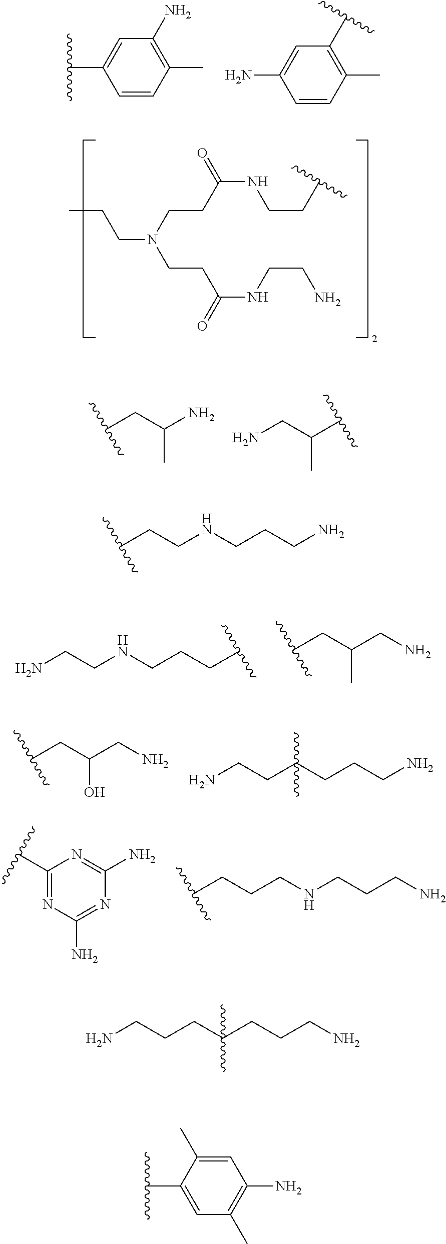 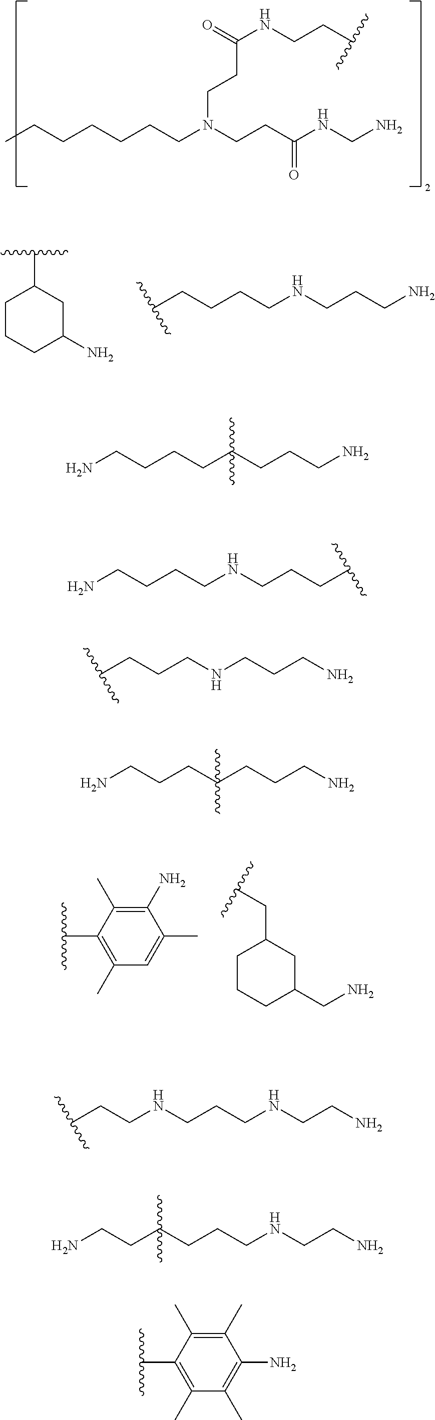 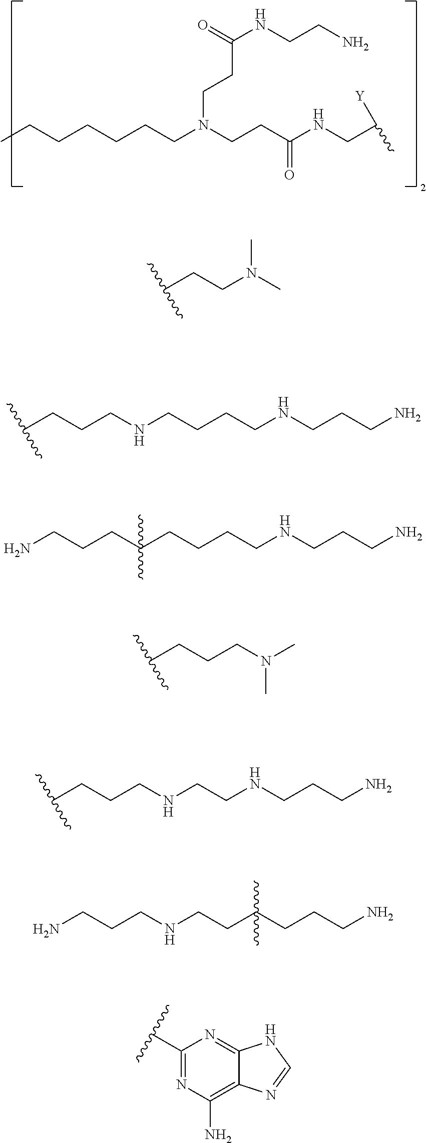 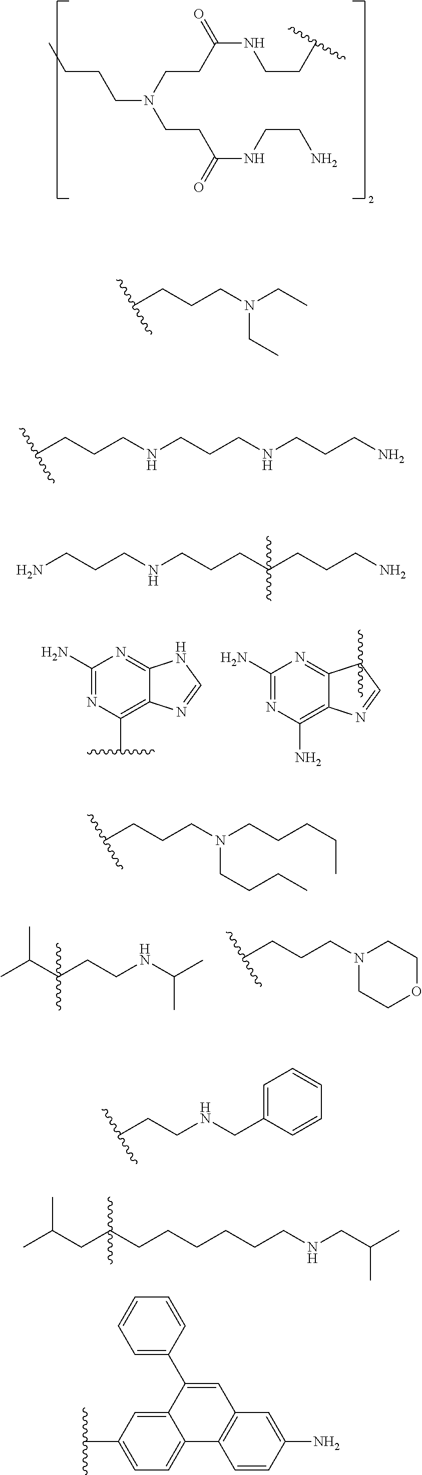 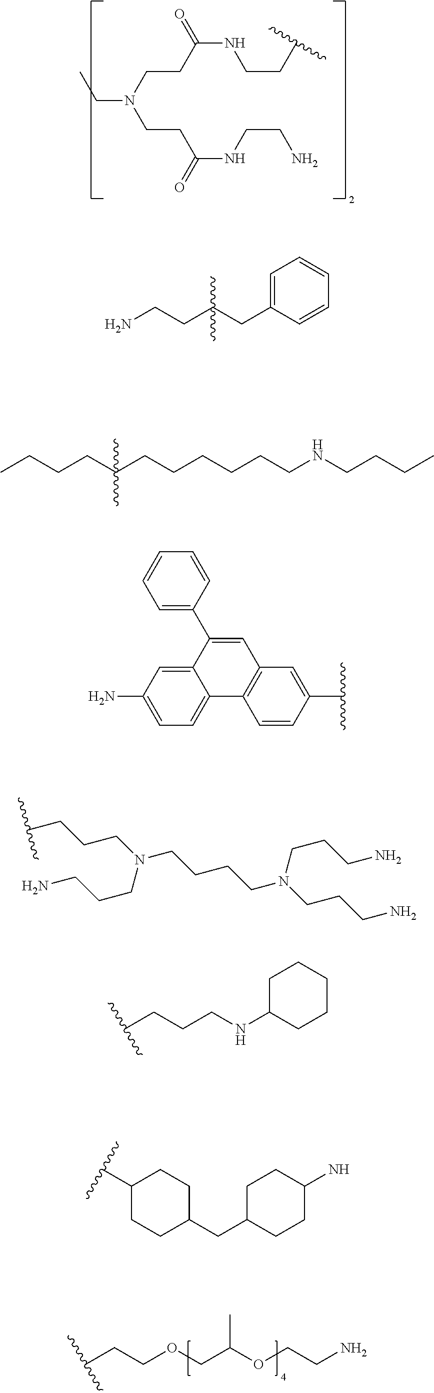 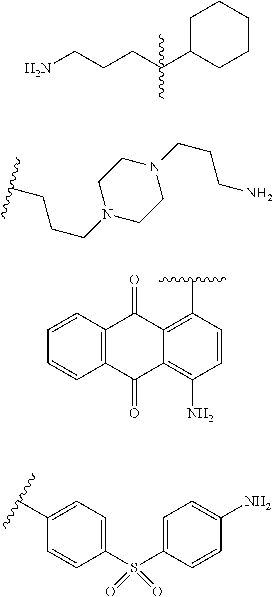 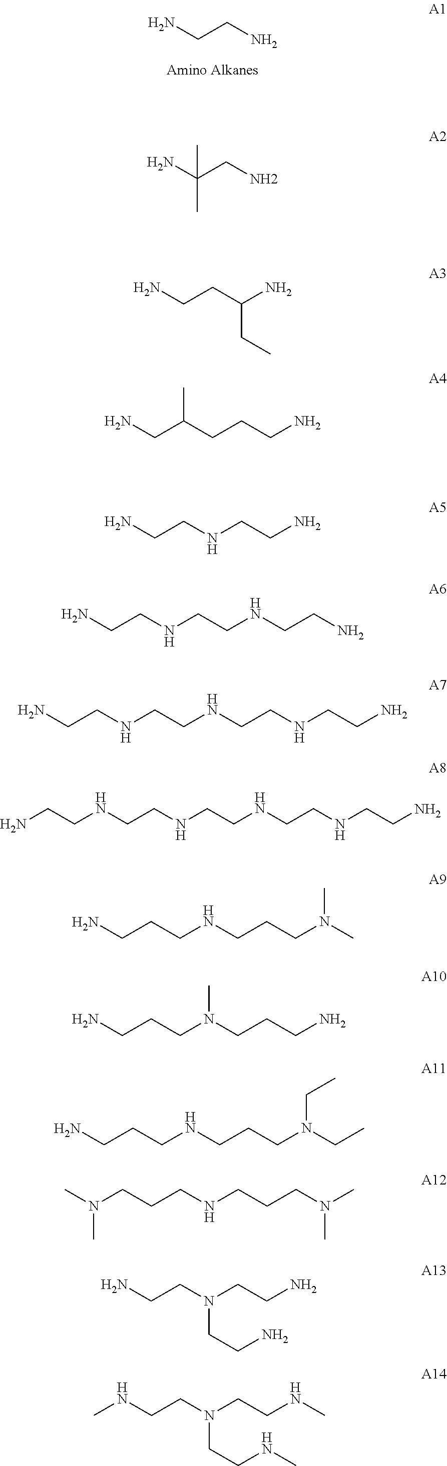 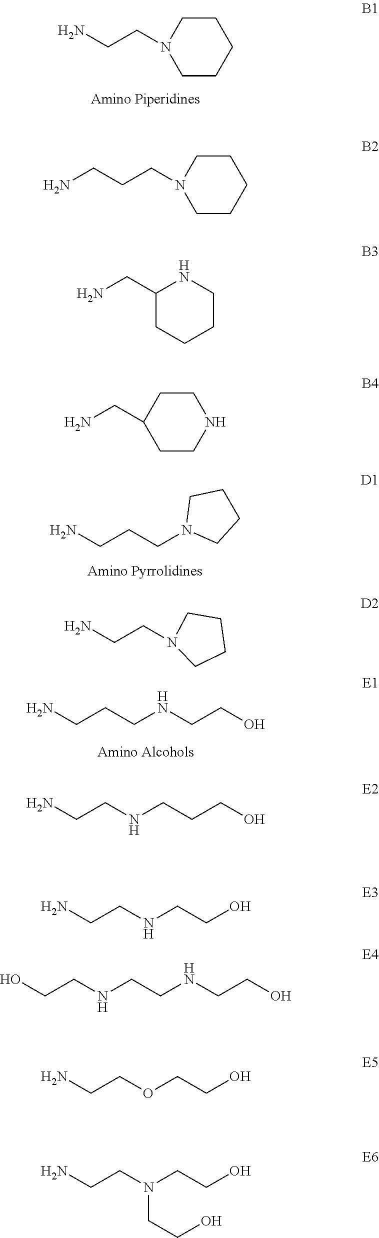 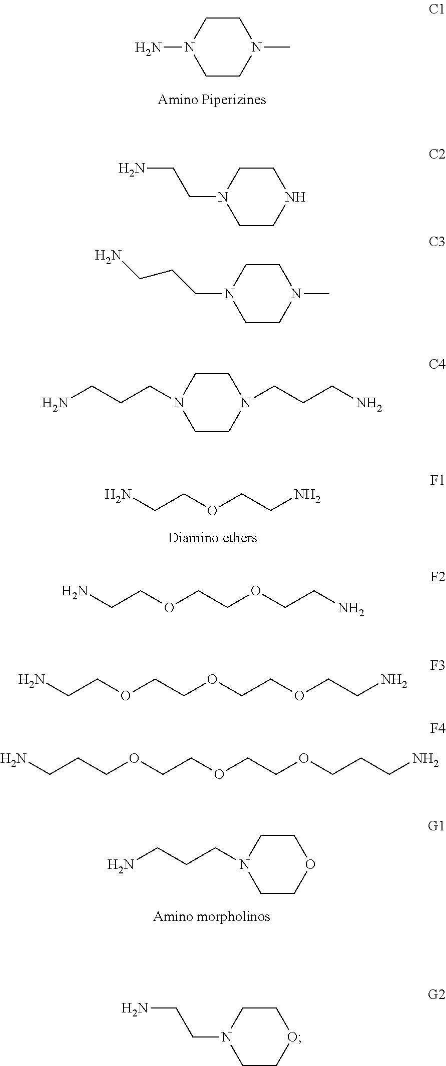 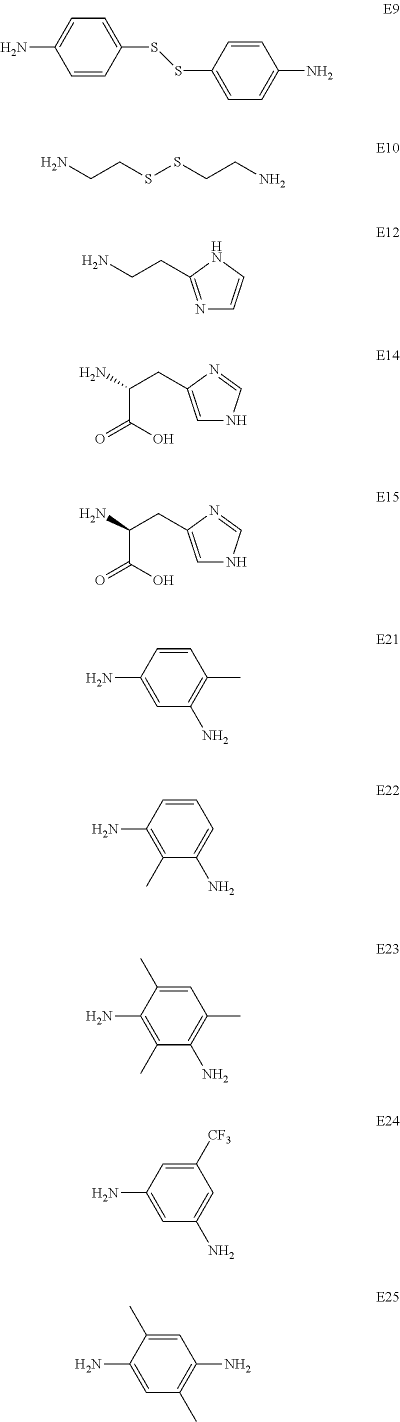 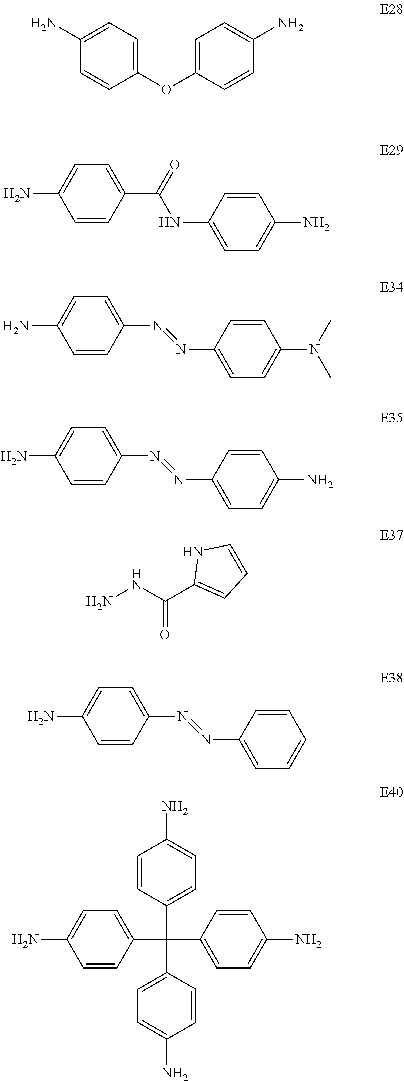 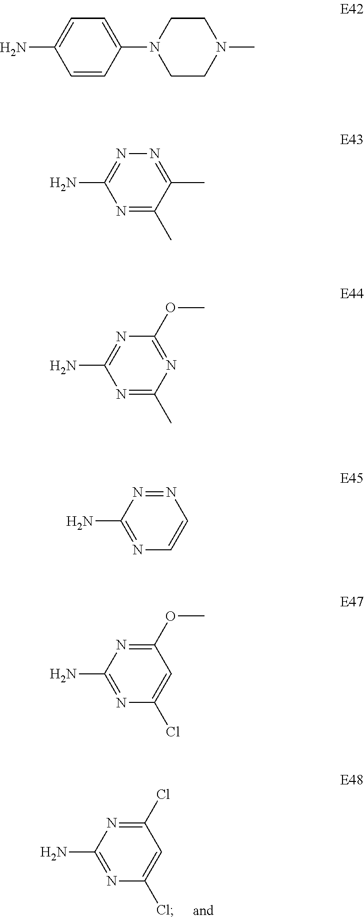
(d) —(CH₂)ₘ₁—(C═C)_{g}(CH₂)ₘ₂—CH₃ is selected from the group consisting of: 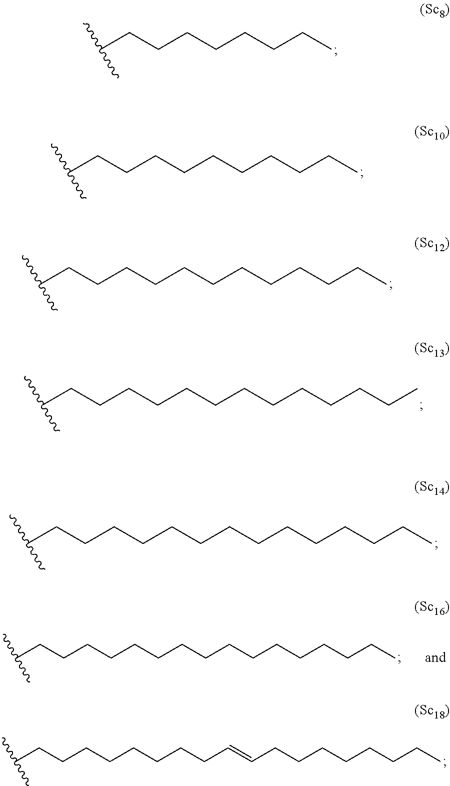
and

3.-5. (canceled)

6. The nanoparticle of claim 2, wherein:
(a) R is selected from the group consisting of: 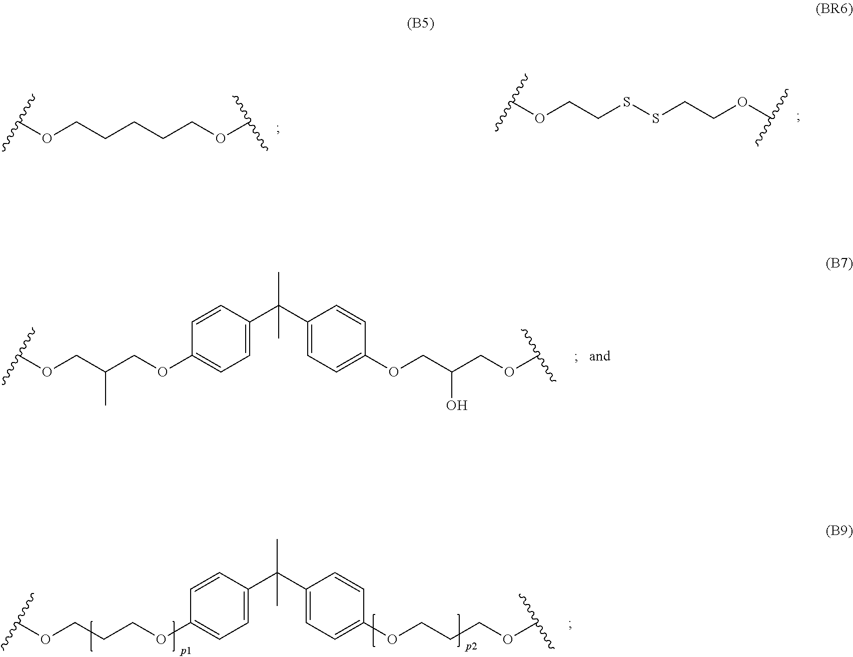
wherein each p1, p2, and t is independently an integer selected from the group consisting of 1, 2, 3, 4, 5, 6, 7, 8, 9, and 10;
(b) R′ is selected from the group consisting of: 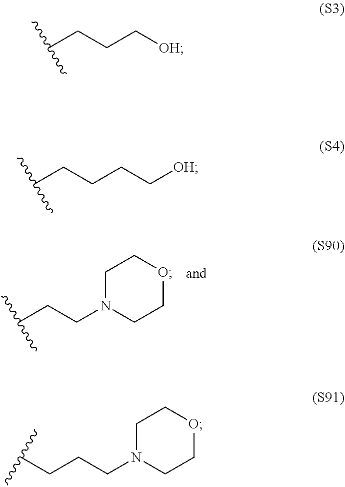 [SMILES: *CCCO ; *CCCCO ; *CCN1CCOCC1 ; *CCCN1CCOCC1]
(c) R″ is selected from the group consisting of: 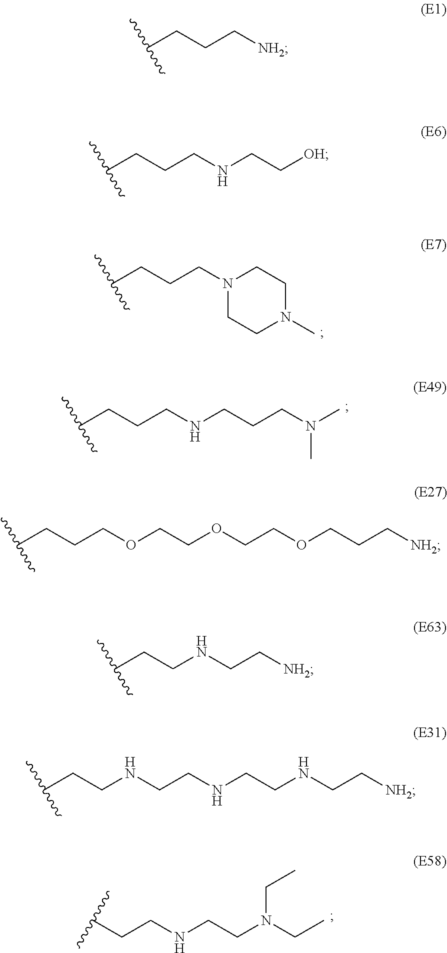 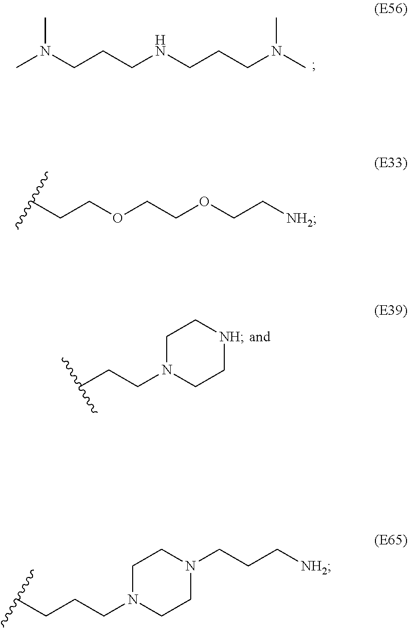
and
(d) —(CH₂)ₘ₁—(C═C)_{q}—(CH₂)ₘ₂—CH₃ is selected from the group consisting of:

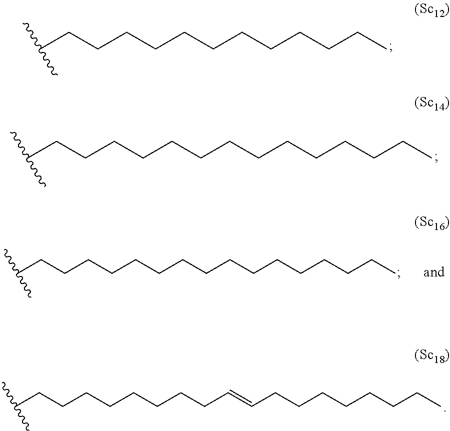 [SMILES: *CCCCCCCCCCCC ; *CCCCCCCCCCCCCC ; *CCCCCCCCCCCCCCCC ; *CCCCCCCC/C=C/CCCCCCCC]

7-9. (canceled)

10. The nanoparticle of claim 2, wherein the compound of formula (I) comprises a combination of:
(a) an R group selected from the group consisting of B5, B7, B9, and BR6;
(b) an R′ group selected from the group consisting of S3, S4, S90, and S91;
(c) an R″ group selected from the group consisting of E1, E6, E7, E27, E31, E33, E39, E49, E56, E58, E63, and E65; and
(d) an —(CH₂)ₘ₁—(C═C)_{q}—(CH₂)ₘ₂—CH₃ moiety selected from the group consisting of Sc12, Sc14, Sc16, and Sc18.

11. The nanoparticle of claim 2, wherein;
(a) R is: 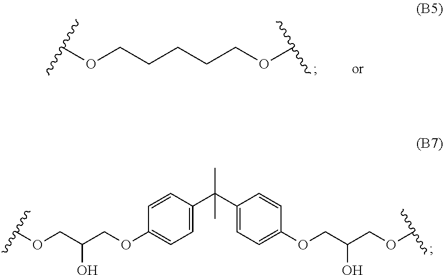 [SMILES: *OCCCCCO* ; *OCC(O)COc1ccc(C(C)(C)c2ccc(OCC(O)CO*)cc2)cc1]
(b) R′ is: 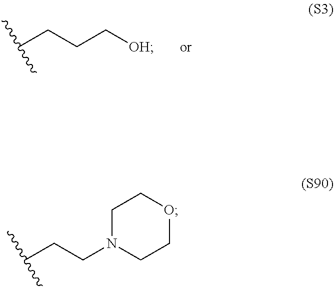 [SMILES: *CCCO ; *CCN1CCOCC1]
(c) R″ is: 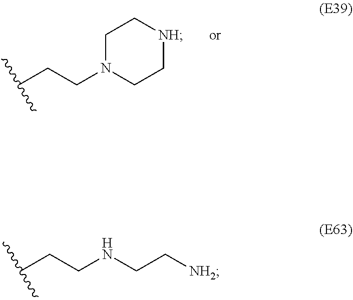 [SMILES: *CCN1CCNCC1 ; *CCNCCN]
and
(d) —(CH₂)ₘ₁—(C═C)_{q}—(CH₂)ₘ₂—CH₃ is Sc12.

12-14. (canceled)

15. The nanoparticle of claim 2, comprising:
B7-S90, Sc12-E63, 50%/50% ratio of S90/Sc16; or
B5-S3, Sc12-E39, 70%/30% ratio of S3/Sc16.

16. The nanoparticle of claim 1, wherein the compound of formula (I) comprises greater than 50% of the dry particle mass.

17. The nanoparticle of claim 1, further comprising one or more additional compounds selected from formula (I) and/or lipid-polyethylene glycol (PEG).

18. The nanoparticle of claim 17, wherein the lipid-PEG is selected from the group consisting of 1,2-dimyristoyl-rac-glycero-3-methoxypolyethylene glycol 2000 (DMG-PEG2k) and C18-PEG2k.

19. (canceled)

20. The nanoparticle of claim 17, comprising a mass percent of lipid-PEG from about 2 wt % to about 10 wt %.

21. The nanoparticle of claim 20, wherein the nanoparticle has a zeta-potential that varies with a weight percent of lipid-PEG, wherein the zeta-potential has a range selected from about −12 mV to about +18 mV or from about −5 mV to about +5 mV.

22. The nanoparticle of claim 1, comprising a plurality of nanoparticles having a polydispersity of less than about 0.2.

23. The nanoparticle of claim 17, wherein the nanoparticle comprises at least three components selected from: (i) one or more of compounds of formula (I), (ii) one or more lipid-PEG, and (iii) one or more nucleic acids.

24. The nanoparticle of claim 1, comprising about a 30:1 ratio of a compound of formula (I) to the one or more nucleic acids.

25. The nanoparticle of claim 1, wherein the one or more nucleic acids is selected from the group consisting of an oligonucleotide, a cyclic dinucleotide, plasmid DNA, linear DNA, siRNA, miRNA, and mRNA.

26. (canceled)

27. The nanoparticle of claim 25, wherein the mRNA comprises self-amplifying mRNA (SAM).

28. The nanoparticle of claim 27, wherein the SAM comprises between about 15,000 to about 20,000 nucleotides.

29. The nanoparticle of claim 27, wherein the mRNA comprises a self-amplifying mRNA (SAM) construct encoding rabies virus glycoprotein.

30. The nanoparticle of claim 1, wherein the nanoparticle further comprises one or more excipients, is lyophilized, and/or comprises a storable powder.

31. The nanoparticle of claim 30, wherein the one or more excipients include one or more cryoprotectants, one or more sugars or sugar alcohols, MgCl₂, and combinations thereof.

32. The nanoparticle of claim 31, wherein:
(a) the one or more cryoprotectants comprise a sugar;
(b) the one or more sugars comprising the one or more excipients are selected from the group consisting of glucose, fructose, sorbitol, mannitol, sucrose, trehalose, and raffinose; and/or
(c) the one or more sugar alcohols comprise sorbitol.

33-36. (canceled)

37. A vaccine comprising a nanoparticle of claim 1.

38. The vaccine of claim 37, wherein the vaccine comprises a vaccine against an infectious disease, wherein the infectious disease is selected from coronavirus, influenza, rabies, Ebola, dengue, polio, and hepatitis.

39. The vaccine of claim 38, wherein the vaccine comprises a rabies vaccine.

40. The vaccine of claim 37, wherein the vaccine comprises a vaccine against cancer or against an autoimmune disease, wherein the autoimmune disease is selected from multiple sclerosis, type 1 diabetes, lupus, celiac disease, colitis, Crohn's disease, and rheumatoid arthritis.

41. A method for delivering one or more nucleic acids to a subject, the method comprising administering a nanoparticle of claim 1 to the subject.

42-46. (canceled)

47. The method of claim 41, wherein the administering is selected from the group consisting of intramuscular, systemic, intranasal, intraocular, and intracranial.

48. The method of claim 47, wherein the administering is intramuscular.

49. The method of claim 46, wherein the SAM reaches a cytosol of a cell of the subject intact.

50. A method for treating or presenting a disease or condition, the method comprising administering a nanoparticle of claim 1 to a subject in need of treatment thereof.

51-56. (canceled)

57. The method of claim 50, wherein the administering is intramuscular.

58. The method of claim 50, wherein the disease or condition is rabies.

59. The method of claim 50, wherein the subject is a human or an animal.

60. A kit comprising one or more of: one or more compounds of formula (I), one or more self-amplifying mRNAs (SAMs), one or more lipid-PEGs, one or more reagents, and instructions for use.
